SEQUENCE LISTING (128 sequences)

SEQ ID NO: 1 - DNA, 558 bp, Homo sapiens
  source 1..558: mol_type = other DNA
caatccgccctcactacaaccggatacgccggtctcgaatgtaatacgactcactataggcacgtagttccggagcttccaccaccgacagctctctgaggccaagggacagctttcgcggctcccgctcgctcagctttcggatgcgggagcccctgtccagcatctccagcgtgcggagcatctgaagttgcagtcttgcgtgtggatggtgcagccaccgggtgcgtgccaggcaggccctcctggggtacaggaagctgtgtgcacgcagcctcgcctgtatggggagcagggaacgggacaggcccccatggtcttcccggtggcctctcggggcttctgacgccaaatgggcttcccatggtcaccctggacaaggaggcaaccaccccacctccacgtagtgcatccccgaccagccccaaggtcttcccgctgagcctctgcagcaccccccaagatgggaacgtggtcgtcgcatgcctggtccagggcttcttcccccagaagagcgatcattcggagacctcccgctactctggcgtcgatgaggga

SEQ ID NO: 2 - RNA, 462 nt, Homo sapiens
  source 1..462: mol_type = other RNA
ggcacgtagttccggagcttccaccaccgacagctctctgaggccaagggacagctttcgcggctcccgctcgctcagctttcggatgcgggagcccctgtccagcatctccagcgtgcggagcatctgaagttgcagtcttgcgtgtggatggtgcagccaccgggtgcgtgccaggcaggccctcctggggtacaggaagctgtgtgcacgcagcctcgcctgtatggggagcagggaacgggacaggcccccatggtcttcccggtggcctctcggggcttctgacgccaaatgggcttcccatggtcaccctggacaaggaggcaaccaccccacctccacgtagtgcatccccgaccagccccaaggtcttcccgctgagcctctgcagcaccccccaagatgggaacgtggtcgtcgcatgcctggtccagggcttcttcccccagaagagcgatc

SEQ ID NO: 3 - DNA, 534 bp, Homo sapiens
  source 1..534: mol_type = other DNA
caatccgccctcactacaaccggatacgccggtctcgaatgtaatacgactcactataggcacgtagtaggaggaatgtaagctggagagaaatcagtctctaaaactgaagaatgacattgaaaatcggcccaagaaggagcaggttctggaactggagcgggagaatgaaatgctgaagaccaaaaaccaggagctgcagtccatcatccaggccgggaagcgcagcctgccagactcagacaaggccatcctggacatcttggaacacgaccgcaaggaggccctggaggacaggcaggagctggtcaacaggatctacaacctgcaggaggaggcccgccaggcagaggagctgcgagacaaggccttccaccacgtagtgcacccaccaaggctccggatgtgttccccatcatatcagggtgcagacacccaaaggataacagccctgtggtcctggcatgcttgataactgggtaccagaagagcgatcattcggagacctcccgctactctggcgtcgatgaggga

SEQ ID NO: 4 - RNA, 438 nt, Homo sapiens
  source 1..438: mol_type = other RNA
ggcacgtagtaggaggaatgtaagctggagagaaatcagtctctaaaactgaagaatgacattgaaaatcggcccaagaaggagcaggttctggaactggagcgggagaatgaaatgctgaagaccaaaaaccaggagctgcagtccatcatccaggccgggaagcgcagcctgccagactcagacaaggccatcctggacatcttggaacacgaccgcaaggaggccctggaggacaggcaggagctggtcaacaggatctacaacctgcaggaggaggcccgccaggcagaggagctgcgagacaaggccttccaccacgtagtgcacccaccaaggctccggatgtgttccccatcatatcagggtgcagacacccaaaggataacagccctgtggtcctggcatgcttgataactgggtaccagaagagcgatc

SEQ ID NO: 5 - DNA, 557 bp, Homo sapiens
  source 1..557: mol_type = other DNA
caatccgccctcactacaaccggatacgccggtctcgaatgtaatacgactcactataggcacgtagttcaggtcctctggtcaatatctgatgggatggtccacatttcctcctcccctggggcaggagggtgtggtagcttgtccaggatggctgaaatcccatggaagttgatggtcatgggattaaccctttcttctctcagagtgtccagcaaccctgcctggccaggattggaatccgtccacagttaagctttgcattctcaaaagtagtacaagctagaccactgagtatgaatgaaccacaaagtccaagggccatttcagtagaaagctcacactgcagagctctggaccttttcacatgctccaggcactgtcctcaggatgtttttccacgtagtgcctccaccaagggcccatcggtcttccccctggtgccctcctccaggagcgtctctgagggcacagcggccctgggctgcctggtcaaggactacttccagaagagcgatcattcggagacctcccgctactctggcgtcgatgaggga

SEQ ID NO: 6 - RNA, 461 nt, Homo sapiens
  source 1..461: mol_type = other RNA
ggcacgtagttcaggtcctctggtcaatatctgatgggatggtccacatttcctcctcccctggggcaggagggtgtggtagcttgtccaggatggctgaaatcccatggaagttgatggtcatgggattaaccctttcttctctcagagtgtccagcaaccctgcctggccaggattggaatccgtccacagttaagctttgcattctcaaaagtagtacaagctagaccactgagtatgaatgaaccacaaagtccaagggccatttcagtagaaagctcacactgcagagctctggaccttttcacatgctccaggcactgtcctcaggatgtttttccacgtagtgcctccaccaagggcccatcggtcttccccctggtgccctcctccaggagcgtctctgagggcacagcggccctgggctgcctggtcaaggactacttccagaagagcgatc

SEQ ID NO: 7 - DNA, 618 bp, Homo sapiens
  source 1..618: mol_type = other DNA
caatccgccctcactacaaccggatacgccggtctcgaatgtaatacgactcactataggcacgtagtttcgtttgatctccagcttggtcccctggccaaaagtgtacggggtactgtaactctgttgacagtagtaagttgcaaaatcttcaggttgcagactgctgatggtgagagtgaaatctgtcccagatccactgccactgaaccttgatgggaccccactttgcaaactggatgcagcatagatcaggagcttaggggctttccctggtttctgctgataccaatttaaatagctgctaatgctctgacttgcccggcaagtgatggtgactctgtctcctacagatgcagacagggaggatggagactgggtcatctggatgtcacatctggcacctcggagccagagtagcaggagccccaggagctgagcggggaccctcatgtccatgctgtgtcctgactgagactccacgtagtcgaactgtggctgcaccatctgtcttcatcttcccgccatctgatgagcagttgaaatctggaactgcctctgttgtgtgagaagagcgatcattcggagacctcccgctactctggcgtcgatgaggga

SEQ ID NO: 8 - RNA, 522 nt, Homo sapiens
  source 1..522: mol_type = other RNA
ggcacgtagtttcgtttgatctccagcttggtcccctggccaaaagtgtacggggtactgtaactctgttgacagtagtaagttgcaaaatcttcaggttgcagactgctgatggtgagagtgaaatctgtcccagatccactgccactgaaccttgatgggaccccactttgcaaactggatgcagcatagatcaggagcttaggggctttccctggtttctgctgataccaatttaaatagctgctaatgctctgacttgcccggcaagtgatggtgactctgtctcctacagatgcagacagggaggatggagactgggtcatctggatgtcacatctggcacctcggagccagagtagcaggagccccaggagctgagcggggaccctcatgtccatgctgtgtcctgactgagactccacgtagtcgaactgtggctgcaccatctgtcttcatcttcccgccatctgatgagcagttgaaatctggaactgcctctgttgtgtgagaagagcgatc

SEQ ID NO: 9 - DNA, 651 bp, Homo sapiens
  source 1..651: mol_type = other DNA
caatccgccctcactacaaccggatacgccggtctcgaatgtaatacgactcactataggcacgtagtcgagggggcagccttgggctgacctaggacggtcagcttggtccctccgccgaataccacagtgctgctgtcccacgcctgacagtaatagtcagcctcatccatagcctgggtcccgctgatggtcagagtggctgtgttcccagagttggagccagagaatcgctcagggatccctgagggccgcttgctatcttgatagatgaccagcacaggggactggcctggcttctgctgataccagcaagcatatttatcccccaatttatctccagagcaggtgatgctggctgtctgtcctggggacacggacactgagggtggctgagtcagctcataggaggccacggatcctgtgcagtaagcaaggacgccgaggaagagagggatccatgccatggctgaggcacctccgatgctgcttcctgaggcccccacgtagtggtcagcccaaggctgccccctcggtcactctgttcccgccctcctctgaggagcttcaagccaacaaggccacactggtgtgtctcataagaagagcgatcattcggagacctcccgctactctggcgtcgatgaggga

SEQ ID NO: 10 - RNA, 555 nt, Homo sapiens
  source 1..555: mol_type = other RNA
ggcacgtagtcgagggggcagccttgggctgacctaggacggtcagcttggtccctccgccgaataccacagtgctgctgtcccacgcctgacagtaatagtcagcctcatccatagcctgggtcccgctgatggtcagagtggctgtgttcccagagttggagccagagaatcgctcagggatccctgagggccgcttgctatcttgatagatgaccagcacaggggactggcctggcttctgctgataccagcaagcatatttatcccccaatttatctccagagcaggtgatgctggctgtctgtcctggggacacggacactgagggtggctgagtcagctcataggaggccacggatcctgtgcagtaagcaaggacgccgaggaagagagggatccatgccatggctgaggcacctccgatgctgcttcctgaggcccccacgtagtggtcagcccaaggctgccccctcggtcactctgttcccgccctcctctgaggagcttcaagccaacaaggccacactggtgtgtctcataagaagagcgatc

SEQ ID NO: 11 - DNA, 551 bp, Homo sapiens
  source 1..551: mol_type = other DNA
caatccgccctcactacaaccggatacgccggtctcgaatgtaatacgactcactataggcacgtagtcatccaagcgagcacctgcttcctattttccgactaatttcggtcccctgatacggaatccgggatcctgcagacaggcagcgggtgacagcaagcacaagggtgttcgatgacacctggagaggtcaggagagccagtcctgcggtgcagaggaagaaacccagcacagaggccaacagagccagccccagcctctggaggcctgaaggtaccgccctgccgcgcgtgggcactgctccctggcgccgtccgtccactgaccatcgtccctcggcccggccgcccgcgcggggcaggcaacgcccgaccgggggcctcggggaccaggaggaacctccacgtagtatatccagaaccctgaccctgccgtgtaccagctgagagactctaaatccagtgacaagtctgtctgcctattcaccgattttgattagaagagcgatcattcggagacctcccgctactctggcgtcgatgaggga

SEQ ID NO: 12 - RNA, 455 nt, Homo sapiens
  source 1..455: mol_type = other RNA
ggcacgtagtcatccaagcgagcacctgcttcctattttccgactaatttcggtcccctgatacggaatccgggatcctgcagacaggcagcgggtgacagcaagcacaagggtgttcgatgacacctggagaggtcaggagagccagtcctgcggtgcagaggaagaaacccagcacagaggccaacagagccagccccagcctctggaggcctgaaggtaccgccctgccgcgcgtgggcactgctccctggcgccgtccgtccactgaccatcgtccctcggcccggccgcccgcgcggggcaggcaacgcccgaccgggggcctcggggaccaggaggaacctccacgtagtatatccagaaccctgaccctgccgtgtaccagctgagagactctaaatccagtgacaagtctgtctgcctattcaccgattttgattagaagagcgatc

SEQ ID NO: 13 - DNA, 627 bp, Homo sapiens
  source 1..627: mol_type = other DNA
caatccgccctcactacaaccggatacgccggtctcgaatgtaatacgactcactataggcacgtagtcttgttcaggtcctctggtcaatatctgatgggatggtccacatttcctcctcccctggggcaggagggtgtggtagcttgtccaggatggctgaaatcccatggaagttgatggtcatgggattaaccctttcttctctcagagtgtccagcaaccctgcctggccaggattggaatccgtccacagttaagctttgcattctcaaaagtagtacaagctagaccactgagtatgaatgaaccacaaagtccaagggccatttcagtagaaagctcacactgcagagctctggaccttttcacatgctccaggcactgtcctcaggatgttttgagctccttagagctggatgtctttgtaaggctgtttggcgaaggggctggtcttctccagtacccacccctctccccacgtagtgaggacctgaacaaggtgttcccacccgaggtcgctgtgtttgagccatcagaagcagagatctcccacacccaaaaggccacactggtgagaagagcgatcattcggagacctcccgctactctggcgtcgatgaggga

SEQ ID NO: 14 - RNA, 531 nt, Homo sapiens
  source 1..531: mol_type = other RNA
ggcacgtagtcttgttcaggtcctctggtcaatatctgatgggatggtccacatttcctcctcccctggggcaggagggtgtggtagcttgtccaggatggctgaaatcccatggaagttgatggtcatgggattaaccctttcttctctcagagtgtccagcaaccctgcctggccaggattggaatccgtccacagttaagctttgcattctcaaaagtagtacaagctagaccactgagtatgaatgaaccacaaagtccaagggccatttcagtagaaagctcacactgcagagctctggaccttttcacatgctccaggcactgtcctcaggatgttttgagctccttagagctggatgtctttgtaaggctgtttggcgaaggggctggtcttctccagtacccacccctctccccacgtagtgaggacctgaacaaggtgttcccacccgaggtcgctgtgtttgagccatcagaagcagagatctcccacacccaaaaggccacactggtgagaagagcgatc

SEQ ID NO: 15 - DNA, 552 bp, Homo sapiens
  source 1..552: mol_type = other DNA
caatccgccctcactacaaccggatacgccggtctcgaatgtaatacgactcactataggcacgtagttgtgacaaagtaacccagagttccccggaccagacggtggcgagtggcagtgaggtggtactgctctgcacttacgacactgtatattcaaatccagatttattctggtaccggataaggccagattattcctttcagtttgtcttttatggggataacagcagatcagaaggtgcagattttactcaaggacggttttctgtgaaacacattctgacccagaaagcctttcacttggtgatctctccagtaaggactgaagacagtgccacttactactgtgcctttagccagacccctgatctttggcaaaggaacctatctggaggtacaacaaccacgtagtgaagtcagcctcataccaaaccatccgtttttgtcatgaaaaatggaacaaatgtcgcttgtctggtgaaggaattctaccccaaggaagaagagcgatcattcggagacctcccgctactctggcgtcgatgaggga

SEQ ID NO: 16 - RNA, 456 nt, Homo sapiens
  source 1..456: mol_type = other RNA
ggcacgtagttgtgacaaagtaacccagagttccccggaccagacggtggcgagtggcagtgaggtggtactgctctgcacttacgacactgtatattcaaatccagatttattctggtaccggataaggccagattattcctttcagtttgtcttttatggggataacagcagatcagaaggtgcagattttactcaaggacggttttctgtgaaacacattctgacccagaaagcctttcacttggtgatctctccagtaaggactgaagacagtgccacttactactgtgcctttagccagacccctgatctttggcaaaggaacctatctggaggtacaacaaccacgtagtgaagtcagcctcataccaaaccatccgtttttgtcatgaaaaatggaacaaatgtcgcttgtctggtgaaggaattctaccccaaggaagaagagcgatc

SEQ ID NO: 17 - DNA, 582 bp, Homo sapiens
  source 1..582: mol_type = other DNA
caatccgccctcactacaaccggatacgccggtctcgaatgtaatacgactcactataggcacgtagtgcaggtcacctagagcaacctcaaatttccagtactaaaacgctgtcaaaaacagcccgcctggaatgtgtggtgtctggaataaaaatttctgcaacatctgtatattggtatcgagagagtcctggtgaagtcatacagttcctggtgtccatttcatatgacggcactgtcagaaaggaatctggcattccgtcaggcaaatttgaggtggataggatacctgaaacgtctacatccactctcaccattcacaatgtagagaaacaggacatagctacctactactgtgccttgtgggaggtgatagtagtgattggatcaagacgtttgcaaaagggactaggctcatagtaacttcgcctgcacgtagtgataaacaacttgatgcagatgtttcccccaagcccactatttttcttccttcgattgctgaaacaaaactccagaaggctggaacatacagaagagcgatcattcggagacctcccgctactctggcgtcgatgaggga

SEQ ID NO: 18 - RNA, 486 nt, Homo sapiens
  source 1..486: mol_type = other RNA
ggcacgtagtgcaggtcacctagagcaacctcaaatttccagtactaaaacgctgtcaaaaacagcccgcctggaatgtgtggtgtctggaataaaaatttctgcaacatctgtatattggtatcgagagagtcctggtgaagtcatacagttcctggtgtccatttcatatgacggcactgtcagaaaggaatctggcattccgtcaggcaaatttgaggtggataggatacctgaaacgtctacatccactctcaccattcacaatgtagagaaacaggacatagctacctactactgtgccttgtgggaggtgatagtagtgattggatcaagacgtttgcaaaagggactaggctcatagtaacttcgcctgcacgtagtgataaacaacttgatgcagatgtttcccccaagcccactatttttcttccttcgattgctgaaacaaaactccagaaggctggaacatacagaagagcgatc

SEQ ID NO: 19 - DNA, 601 bp, Homo sapiens
  source 1..601: mol_type = other DNA
caatccgccctcactacaaccggatacgccggtctcgaatgtaatacgactcactataggcacgtagtcaggtcaccttgagggagtctggtcctgcgctggtgaaacccacacagaccctcacactgacctgcaccttctctgggttctcactcagcactagtggaatgtgtgtgagctgggtccgtcagcccccagggaaggccctggagtggcttgcactcattgattgggatgatgataaatactacagcacatctctgaagaccaggctcaccatctccaaggacacctccaaaaaccaggtggtccttacaatgaccaacatggaccctgtggacacagccacgtattactgtgcacggatacaagcgacgcgcagtggatacagctatggttacacgacaactggttcgacccctggggccagggaaccctggtcaccgtctcctcagcacgtagtgggagtgcatccgccccaacccttttccccctcgtctcctgtgagaattccccgtcggatacgagcagcgtggccgttggctgcctcgcacaggacttccattcggagacctcccgctactctggcgtcgatgaggga

SEQ ID NO: 20 - RNA, 505 nt, Homo sapiens
  source 1..505: mol_type = other RNA
ggcacgtagtcaggtcaccttgagggagtctggtcctgcgctggtgaaacccacacagaccctcacactgacctgcaccttctctgggttctcactcagcactagtggaatgtgtgtgagctgggtccgtcagcccccagggaaggccctggagtggcttgcactcattgattgggatgatgataaatactacagcacatctctgaagaccaggctcaccatctccaaggacacctccaaaaaccaggtggtccttacaatgaccaacatggaccctgtggacacagccacgtattactgtgcacggatacaagcgacgcgcagtggatacagctatggttacacgacaactggttcgacccctggggccagggaaccctggtcaccgtctcctcagcacgtagtgggagtgcatccgccccaacccttttccccctcgtctcctgtgagaattccccgtcggatacgagcagcgtggccgttggctgcctcgcacaggacttcc

SEQ ID NO: 21 - DNA, 639 bp, Homo sapiens
  source 1..639: mol_type = other DNA
caatccgccctcactacaaccggatacgccggtctcgaatgtaatacgactcactataggcacgtagtcaggtcaccttgaaggagtctggtcctgcgctggtgaaacccacacagaccctcacactgacctgcaccttctctgggttctcactcagcactagtggaatgcgtgcgagctggatccgtcagcccccagggaaggccctggagtggcttgcacgcattgattgggatgatgataaattctacagcacatctctgaagaccaggctcaccatctccaaggacacctccaaaaaccaggtggtccttacaatgaccaacatggaccctgtggacacagccacgtattactgtgcacggataccaggcgacaacagacccgtattatgattacatttgggggagttatcgttataccaccagacattactactactactacggtatggacgtctggggcaaagggaccacggtcaccgtctcctcagcacgtagtgggagtgcatccgccccaacccttttccccctcgtctcctgtgagaattccccgtcggatacgagcagcgtggccgttggctgcctcgcacaggacttccattcggagacctcccgctactctggcgtcgatgaggga

SEQ ID NO: 22 - RNA, 543 nt, Homo sapiens
  source 1..543: mol_type = other RNA
ggcacgtagtcaggtcaccttgaaggagtctggtcctgcgctggtgaaacccacacagaccctcacactgacctgcaccttctctgggttctcactcagcactagtggaatgcgtgcgagctggatccgtcagcccccagggaaggccctggagtggcttgcacgcattgattgggatgatgataaattctacagcacatctctgaagaccaggctcaccatctccaaggacacctccaaaaaccaggtggtccttacaatgaccaacatggaccctgtggacacagccacgtattactgtgcacggataccaggcgacaacagacccgtattatgattacatttgggggagttatcgttataccaccagacattactactactactacggtatggacgtctggggcaaagggaccacggtcaccgtctcctcagcacgtagtgggagtgcatccgccccaacccttttccccctcgtctcctgtgagaattccccgtcggatacgagcagcgtggccgttggctgcctcgcacaggacttcc

SEQ ID NO: 23 - DNA, 607 bp, Homo sapiens
  source 1..607: mol_type = other DNA
caatccgccctcactacaaccggatacgccggtctcgaatgtaatacgactcactataggcacgtagtgaggtccagctggtacagtctggggctgaggtgaagaagcctggggctacagtgaaaatctcctgcaaggtttctggatacaccttcaccgactactacatgcactgggtgcaacaggcccctggaaaagggcttgagtggatgggacttgttgatcctgaagatggtgaaacaatatacgcagagaagttccagggcagagtcaccataaccgcggacacgtctacagacacagcctacatggagctgagcagcctgagatctgaggacacggccgtgtattactgtgcaacagaagcaaaagccctatgtaacagaccatggccccatgaaagcggagcccaaactactttgactactggggccaaggaaccctggtcaccgtctcctcagcacgtagtgcacccaccaaggctccggatgtgttccccatcatatcagggtgcagacacccaaaggataacagccctgtggtcctggcatgcttgataactgggtaccattcggagacctcccgctactctggcgtcgatgaggga

SEQ ID NO: 24 - RNA, 511 nt, Homo sapiens
  source 1..511: mol_type = other RNA
ggcacgtagtgaggtccagctggtacagtctggggctgaggtgaagaagcctggggctacagtgaaaatctcctgcaaggtttctggatacaccttcaccgactactacatgcactgggtgcaacaggcccctggaaaagggcttgagtggatgggacttgttgatcctgaagatggtgaaacaatatacgcagagaagttccagggcagagtcaccataaccgcggacacgtctacagacacagcctacatggagctgagcagcctgagatctgaggacacggccgtgtattactgtgcaacagaagcaaaagccctatgtaacagaccatggccccatgaaagcggagcccaaactactttgactactggggccaaggaaccctggtcaccgtctcctcagcacgtagtgcacccaccaaggctccggatgtgttccccatcatatcagggtgcagacacccaaaggataacagccctgtggtcctggcatgcttgataactgggtacc

SEQ ID NO: 25 - DNA, 677 bp, Homo sapiens
  source 1..677: mol_type = other DNA
caatccgccctcactacaaccggatacgccggtctcgaatgtaatacgactcactataggcacgtagtcaggtgcagctgcaggagtcgggcccaggactggtgaagccttcacagaccctgtccctcacctgcactgtctctggtggctccatcagcagtggtagttactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactgacaaggcacgaccgagtctccaaaggcacaggcctcctcagtgcccactgcacaaccccaagcagagggcagcctcgccaaggcaaccacagccccagccaccacccgtaacacaggggcggctgatgcttttgatatctggggccaagggacaatggtcaccgtctcttcagcacgtagtgcacccaccaaggctccggatgtgttccccatcatatcagggtgcagacacccaaaggataacagccctgtggtcctggcatgcttgataactgggtaccattcggagacctcccgctactctggcgtcgatgaggga

SEQ ID NO: 26 - RNA, 581 nt, Homo sapiens
  source 1..581: mol_type = other RNA
ggcacgtagtcaggtgcagctgcaggagtcgggcccaggactggtgaagccttcacagaccctgtccctcacctgcactgtctctggtggctccatcagcagtggtagttactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactgacaaggcacgaccgagtctccaaaggcacaggcctcctcagtgcccactgcacaaccccaagcagagggcagcctcgccaaggcaaccacagccccagccaccacccgtaacacaggggcggctgatgcttttgatatctggggccaagggacaatggtcaccgtctcttcagcacgtagtgcacccaccaaggctccggatgtgttccccatcatatcagggtgcagacacccaaaggataacagccctgtggtcctggcatgcttgataactgggtacc

SEQ ID NO: 27 - DNA, 661 bp, Homo sapiens
  source 1..661: mol_type = other DNA
caatccgccctcactacaaccggatacgccggtctcgaatgtaatacgactcactataggcacgtagttctggtggctccgtcagcagtggtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagacacacccaatacctggccatgacccccctgatccctcagagcaaggatgagaacagcgatgactacacgacctttgatgatgtgggcagcctgtggaccaccctgtccacgtttgtggccctcttcatcctcaccctcctctacagcggcattgtcactttcatcaagacaacaactggttcgactcctggggccaaggaaccctggtcaccgtctcctcagcacgtagtgcatccccgaccagccccaaggtcttcccgctgagcctctgcagcaccccccaagatgggaacgtggtcgtcgcatgcctggtccagggcttcttcccccattcggagacctcccgctactctggcgtcgatgaggga

SEQ ID NO: 28 - RNA, 565 nt, Homo sapiens
  source 1..565: mol_type = other RNA
ggcacgtagttctggtggctccgtcagcagtggtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagacacacccaatacctggccatgacccccctgatccctcagagcaaggatgagaacagcgatgactacacgacctttgatgatgtgggcagcctgtggaccaccctgtccacgtttgtggccctcttcatcctcaccctcctctacagcggcattgtcactttcatcaagacaacaactggttcgactcctggggccaaggaaccctggtcaccgtctcctcagcacgtagtgcatccccgaccagccccaaggtcttcccgctgagcctctgcagcaccccccaagatgggaacgtggtcgtcgcatgcctggtccagggcttcttccccc

SEQ ID NO: 29 - DNA, 620 bp, Homo sapiens
  source 1..620: mol_type = other DNA
caatccgccctcactacaaccggatacgccggtctcgaatgtaatacgactcactataggcacgtagtcagatcaccttgaaggagtctggtcctacgctggtgaaacccacacagaccctcacgctgacccgcaccttctctgggttctcactcagcactagtggaatgtgtgtgagctggatccgtcagcccccagggaaggccctggagtggcttgcactcattgattgggatgatgataaatactacagcacatctctgaacaccaggctcaccatctccaaggacacctccaaaaaccaggtggtccttacaatgaccaacatggaccctgtggacacaggcacatattactgtgtacggagcgcaaacaccgaggtattagcatttttggagtggttattatacccaacacaacgcaacaactggttcgacccctggggccagggaaccctggtcaccgtctcctcagcacgtagtgcatccccgaccagccccaaggtcttcccgctgagcctctgcagcaccccccaagatgggaacgtggtcgtcgcatgcctggtccagggcttcttcccccattcggagacctcccgctactctggcgtcgatgaggga

SEQ ID NO: 30 - RNA, 524 nt, Homo sapiens
  source 1..524: mol_type = other RNA
ggcacgtagtcagatcaccttgaaggagtctggtcctacgctggtgaaacccacacagaccctcacgctgacccgcaccttctctgggttctcactcagcactagtggaatgtgtgtgagctggatccgtcagcccccagggaaggccctggagtggcttgcactcattgattgggatgatgataaatactacagcacatctctgaacaccaggctcaccatctccaaggacacctccaaaaaccaggtggtccttacaatgaccaacatggaccctgtggacacaggcacatattactgtgtacggagcgcaaacaccgaggtattagcatttttggagtggttattatacccaacacaacgcaacaactggttcgacccctggggccagggaaccctggtcaccgtctcctcagcacgtagtgcatccccgaccagccccaaggtcttcccgctgagcctctgcagcaccccccaagatgggaacgtggtcgtcgcatgcctggtccagggcttcttccccc

SEQ ID NO: 31 - DNA, 615 bp, Homo sapiens
  source 1..615: mol_type = other DNA
caatccgccctcactacaaccggatacgccggtctcgaatgtaatacgactcactataggcacgtagtgaggtgcagctggtggagtctgggggaggcttggtacagcctggggggtccctgagactctcctgtgcagcctctggattcaccttcagtaacagtgacatgaactgggcccgcaaggctccaggaaaggggctggagtgggtatcgggtgttagttggaatggcagtaggacgcactatgtggactccgtgaagcgccgattcatcatctccagagacaattccaggaactccctgtatctgcaaaagaacagacggagagccgaggacatggctgtgtattactgtgtgagaaaccgcaggagagcagtattatgatttttggactggttattatacccgggggcagggtgatgcttttgatgtctggggccaagggacaatggtcaccgtctcttcagcacgtagtgcctccacacagagcccatccgtcttccccttgacccgctgctgcaaaaacattccctccaatgccacctccgtgactctgggctgcctggccacgggctattcggagacctcccgctactctggcgtcgatgaggga

SEQ ID NO: 32 - RNA, 519 nt, Homo sapiens
  source 1..519: mol_type = other RNA
ggcacgtagtgaggtgcagctggtggagtctgggggaggcttggtacagcctggggggtccctgagactctcctgtgcagcctctggattcaccttcagtaacagtgacatgaactgggcccgcaaggctccaggaaaggggctggagtgggtatcgggtgttagttggaatggcagtaggacgcactatgtggactccgtgaagcgccgattcatcatctccagagacaattccaggaactccctgtatctgcaaaagaacagacggagagccgaggacatggctgtgtattactgtgtgagaaaccgcaggagagcagtattatgatttttggactggttattatacccgggggcagggtgatgcttttgatgtctggggccaagggacaatggtcaccgtctcttcagcacgtagtgcctccacacagagcccatccgtcttccccttgacccgctgctgcaaaaacattccctccaatgccacctccgtgactctgggctgcctggccacgggct

SEQ ID NO: 33 - DNA, 645 bp, Homo sapiens
  source 1..645: mol_type = other DNA
caatccgccctcactacaaccggatacgccggtctcgaatgtaatacgactcactataggcacgtagtgaggtgcagctggtgcagtctggagcagaggtgaaaaagcccggggagtctctgaagatctcctgtaagggttctggatacagctttaccagctactggatcggctgggtgcaccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacaagaggaaggaagaggaggagaagagaagaagaaggagaaggagaaagaggaacaagaagagagagagacaaagacaccagaaacacaactggttcgacccctggggccagggaaccctggtcaccgtctcctcagcacgtagtgcctccacacagagcccatccgtcttccccttgacccgctgctgcaaaaacattccctccaatgccacctccgtgactctgggctgcctggccacgggctattcggagacctcccgctactctggcgtcgatgaggga

SEQ ID NO: 34 - RNA, 549 nt, Homo sapiens
  source 1..549: mol_type = other RNA
ggcacgtagtgaggtgcagctggtgcagtctggagcagaggtgaaaaagcccggggagtctctgaagatctcctgtaagggttctggatacagctttaccagctactggatcggctgggtgcaccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacaagaggaaggaagaggaggagaagagaagaagaaggagaaggagaaagaggaacaagaagagagagagacaaagacaccagaaacacaactggttcgacccctggggccagggaaccctggtcaccgtctcctcagcacgtagtgcctccacacagagcccatccgtcttccccttgacccgctgctgcaaaaacattccctccaatgccacctccgtgactctgggctgcctggccacgggct

SEQ ID NO: 35 - DNA, 649 bp, Homo sapiens
  source 1..649: mol_type = other DNA
caatccgccctcactacaaccggatacgccggtctcgaatgtaatacgactcactataggcacgtagtcaggtccagctggtgcagtctggggctgaggtgaagaagcctggggcctcagtgaaggtttcctgcaaggcttctggatacaccttcactagctatgctatgcattgggtgcgccaggcccccggacaaaggcttgagtggatgggatggatcaacgctggcaatggtaacacaaaatattcacaggagttccagggcagagtcaccattaccagggacacatccgcgagcacagcctacatggagctgagcagcctgagatctgaggacatggctgtgtattactgtgcgagagacagcagcagccgggaagaggaggagaagagaagaagaaggagaaggagaaagaggaacaagaagagagagagacaaagacaccaagaagtgatgcttttgatatctggggccaagggacaatggtcaccgtctcttcagcacgtagtgcctccaccaagggcccatcggtcttccccctggtgccctcctccaggagcgtctctgagggcacagcggccctgggctgcctggtcaaggactacttccattcggagacctcccgctactctggcgtcgatgaggga

SEQ ID NO: 36 - RNA, 553 nt, Homo sapiens
  source 1..553: mol_type = other RNA
ggcacgtagtcaggtccagctggtgcagtctggggctgaggtgaagaagcctggggcctcagtgaaggtttcctgcaaggcttctggatacaccttcactagctatgctatgcattgggtgcgccaggcccccggacaaaggcttgagtggatgggatggatcaacgctggcaatggtaacacaaaatattcacaggagttccagggcagagtcaccattaccagggacacatccgcgagcacagcctacatggagctgagcagcctgagatctgaggacatggctgtgtattactgtgcgagagacagcagcagccgggaagaggaggagaagagaagaagaaggagaaggagaaagaggaacaagaagagagagagacaaagacaccaagaagtgatgcttttgatatctggggccaagggacaatggtcaccgtctcttcagcacgtagtgcctccaccaagggcccatcggtcttccccctggtgccctcctccaggagcgtctctgagggcacagcggccctgggctgcctggtcaaggactacttcc

SEQ ID NO: 37 - DNA, 613 bp, Homo sapiens
  source 1..613: mol_type = other DNA
caatccgccctcactacaaccggatacgccggtctcgaatgtaatacgactcactataggcacgtagtcaggtcaccttgaaggagtctggtcctgtgctggtgaaacccacacagaccctcacactgacctgcaccttctctgggttctcactcagcactagtggaatgtgtgtgagctggatccgtcagcccccagggaaggccctggagtggcttgcacgcattgattgggatgatgataaattctacagcacatctctgaagaccaggctcaccatctccaaggacacctccaaaaaccaggtggtccttacaatgaccaacatggaccctgtggacacagccacgtattactgtgcacggataccagcagagcgcaacacatgactacagtaactacagggcgggacgggactgatgcttttgatgtctggggccaagggacaatggtcaccgtctcttcagcacgtagtgcctccaccaagggcccatcggtcttccccctggtgccctcctccaggagcgtctctgagggcacagcggccctgggctgcctggtcaaggactacttccattcggagacctcccgctactctggcgtcgatgaggga

SEQ ID NO: 38 - RNA, 517 nt, Homo sapiens
  source 1..517: mol_type = other RNA
ggcacgtagtcaggtcaccttgaaggagtctggtcctgtgctggtgaaacccacacagaccctcacactgacctgcaccttctctgggttctcactcagcactagtggaatgtgtgtgagctggatccgtcagcccccagggaaggccctggagtggcttgcacgcattgattgggatgatgataaattctacagcacatctctgaagaccaggctcaccatctccaaggacacctccaaaaaccaggtggtccttacaatgaccaacatggaccctgtggacacagccacgtattactgtgcacggataccagcagagcgcaacacatgactacagtaactacagggcgggacgggactgatgcttttgatgtctggggccaagggacaatggtcaccgtctcttcagcacgtagtgcctccaccaagggcccatcggtcttccccctggtgccctcctccaggagcgtctctgagggcacagcggccctgggctgcctggtcaaggactacttcc

SEQ ID NO: 39 - DNA, 549 bp, Homo sapiens
  source 1..549: mol_type = other DNA
caatccgccctcactacaaccggatacgccggtctcgaatgtaatacgactcactataggcacgtagtgacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctccgccaccccgcgcgctcacgttcggcggagggaccaaggtggagatcaaaccacgtagtcgaactgtggctgcaccatctgtcttcatcttcccgccatctgatgagcagttgaaatctggaactgcctctgttgtgtgattcggagacctcccgctactctggcgtcgatgaggga

SEQ ID NO: 40 - RNA, 453 nt, Homo sapiens
  source 1..453: mol_type = other RNA
ggcacgtagtgacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctccgccaccccgcgcgctcacgttcggcggagggaccaaggtggagatcaaaccacgtagtcgaactgtggctgcaccatctgtcttcatcttcccgccatctgatgagcagttgaaatctggaactgcctctgttgtgtg

SEQ ID NO: 41 - DNA, 535 bp, Homo sapiens
  source 1..535: mol_type = other DNA
caatccgccctcactacaaccggatacgccggtctcgaatgtaatacgactcactataggcacgtagtgaaacgacactcacgcagtctccagcattcatgtcagcgactccaggagacaaagtcaacatctcctgcaaagccagccaagacattgatgatgatatgaactggtaccaacagaaaccaggagaagctgctattttcattattcaagaagctactactctcgttcctggaatcccacctcgattcagtggcagcgggtatggaacagattttaccctcacaattaataacatagaatctgaggatgctgcatattacttctgtctacaacatgataatttccctctccaggaacaaccgcaggtgcacttttggccaggggaccaagctggagatcaaaccacgtagtcgaactgtggctgcaccatctgtcttcatcttcccgccatctgatgagcagttgaaatctggaactgcctctgttgtgtgattcggagacctcccgctactctggcgtcgatgaggga

SEQ ID NO: 42 - RNA, 439 nt, Homo sapiens
  source 1..439: mol_type = other RNA
ggcacgtagtgaaacgacactcacgcagtctccagcattcatgtcagcgactccaggagacaaagtcaacatctcctgcaaagccagccaagacattgatgatgatatgaactggtaccaacagaaaccaggagaagctgctattttcattattcaagaagctactactctcgttcctggaatcccacctcgattcagtggcagcgggtatggaacagattttaccctcacaattaataacatagaatctgaggatgctgcatattacttctgtctacaacatgataatttccctctccaggaacaaccgcaggtgcacttttggccaggggaccaagctggagatcaaaccacgtagtcgaactgtggctgcaccatctgtcttcatcttcccgccatctgatgagcagttgaaatctggaactgcctctgttgtgtg

SEQ ID NO: 43 - DNA, 548 bp, Homo sapiens
  source 1..548: mol_type = other DNA
caatccgccctcactacaaccggatacgccggtctcgaatgtaatacgactcactataggcacgtagtcagtctgtgctgactcagccaccctcagcgtctgggacccccgggcagagggtcaccatctcttgttctggaagcagctccaacatcggaagtaattatgtatactggtaccagcagctcccaggaacggcccccaaactcctcatctataggaataatcagcggccctcaggggtccctgaccgattctctggctccaagtctggcacctcagcctccctggccatcagtgggctctggtccgaggatgaggctgattattactgtgcagcatgggatgacagcctgagtggtccaaaaccgcacagcacatgtttggcagcaagacccagcccactgtcttacacgtagtggtcagcccaaggctgccccctcggtcactctgttcccgccctcctctgaggagcttcaagccaacaaggccacactggtgtgtctcataattcggagacctcccgctactctggcgtcgatgaggga

SEQ ID NO: 44 - RNA, 452 nt, Homo sapiens
  source 1..452: mol_type = other RNA
ggcacgtagtcagtctgtgctgactcagccaccctcagcgtctgggacccccgggcagagggtcaccatctcttgttctggaagcagctccaacatcggaagtaattatgtatactggtaccagcagctcccaggaacggcccccaaactcctcatctataggaataatcagcggccctcaggggtccctgaccgattctctggctccaagtctggcacctcagcctccctggccatcagtgggctctggtccgaggatgaggctgattattactgtgcagcatgggatgacagcctgagtggtccaaaaccgcacagcacatgtttggcagcaagacccagcccactgtcttacacgtagtggtcagcccaaggctgccccctcggtcactctgttcccgccctcctctgaggagcttcaagccaacaaggccacactggtgtgtctcata

SEQ ID NO: 45 - DNA, 563 bp, Homo sapiens
  source 1..563: mol_type = other DNA
caatccgccctcactacaaccggatacgccggtctcgaatgtaatacgactcactataggcacgtagtcagcttgtgctgactcaatcgccctctgcctctgcctccctgggagcctcggtcaagctcacctgcactctgagcagtgggcacagcagctacgccatcgcatggcatcagcagcagccagagaagggccctcggtacttgatgaagcttaacagtgatggcagccacagtaagggggacgggatccctgatcgcttctcaggctccagctctggggctgagcgctacctcaccatctccagcctccagtctgaggatgaggctgactattactgtcagacctggggcactggcattcaaaaggccagcacgacggggagaggcacatgtttggcagcaagacccagcccactgtcttacacgtagtggtcagcccaaggctgccccctcggtcactctgttcccgccctcctctgaggagcttcaagccaacaaggccacactggtgtgtctcataattcggagacctcccgctactctggcgtcgatgaggga

SEQ ID NO: 46 - RNA, 467 nt, Homo sapiens
  source 1..467: mol_type = other RNA
ggcacgtagtcagcttgtgctgactcaatcgccctctgcctctgcctccctgggagcctcggtcaagctcacctgcactctgagcagtgggcacagcagctacgccatcgcatggcatcagcagcagccagagaagggccctcggtacttgatgaagcttaacagtgatggcagccacagtaagggggacgggatccctgatcgcttctcaggctccagctctggggctgagcgctacctcaccatctccagcctccagtctgaggatgaggctgactattactgtcagacctggggcactggcattcaaaaggccagcacgacggggagaggcacatgtttggcagcaagacccagcccactgtcttacacgtagtggtcagcccaaggctgccccctcggtcactctgttcccgccctcctctgaggagcttcaagccaacaaggccacactggtgtgtctcata

SEQ ID NO: 47 - DNA, 552 bp, Homo sapiens
  source 1..552: mol_type = other DNA
caatccgccctcactacaaccggatacgccggtctcgaatgtaatacgactcactataggcacgtagtgcccagagagtgactcagcccgagaagctcctctctgtctttaaaggggccccagtggagctgaagtgcaactattcctattctgggagtcctgaactcttctggtatgtccagtactccagacaacgcctccagttactcttgagacacatctctagagagagcatcaaaggcttcactgctgaccttaacaaaggcgagacatctttccacctgaagaaaccatttgctcaagaggaagactcagccatgtattactgtgctctaagtggccgagggaagcgaccagagtaattcagggagcccagaagctggtatttggccaaggaaccaggctgactatcaacccaacacgtagtatatccagaaccctgaccctgccgtgtaccagctgagagactctaaatccagtgacaagtctgtctgcctattcaccgattttgattattcggagacctcccgctactctggcgtcgatgaggga

SEQ ID NO: 48 - RNA, 456 nt, Homo sapiens
  source 1..456: mol_type = other RNA
ggcacgtagtgcccagagagtgactcagcccgagaagctcctctctgtctttaaaggggccccagtggagctgaagtgcaactattcctattctgggagtcctgaactcttctggtatgtccagtactccagacaacgcctccagttactcttgagacacatctctagagagagcatcaaaggcttcactgctgaccttaacaaaggcgagacatctttccacctgaagaaaccatttgctcaagaggaagactcagccatgtattactgtgctctaagtggccgagggaagcgaccagagtaattcagggagcccagaagctggtatttggccaaggaaccaggctgactatcaacccaacacgtagtatatccagaaccctgaccctgccgtgtaccagctgagagactctaaatccagtgacaagtctgtctgcctattcaccgattttgatt

SEQ ID NO: 49 - DNA, 558 bp, Homo sapiens
  source 1..558: mol_type = other DNA
caatccgccctcactacaaccggatacgccggtctcgaatgtaatacgactcactataggcacgtagtaaaaaccaagtggagcagagtcctcagtccctgatcatcctggagggaaagaactgcactcttcaatgcaattatacagtgagccccttcagcaacttaaggtggtataagcaagatactgggagaggtcctgtttccctgacaatcatgactttcagtgagaacacaaagtcgaacggaagatatacagcaactctggatgcagacacaaagcaaagctctctgcacatcacagcctcccagctcagcgattcagcctcctacatctgtgtggtgagcgagcagcgaccgggatgacaactgacagctgggggaaattgcagtttggagcagggacccaggttgtggtcaccccagcacgtagtatatccagaaccctgaccctgccgtgtaccagctgagagactctaaatccagtgacaagtctgtctgcctattcaccgattttgattattcggagacctcccgctactctggcgtcgatgaggga

SEQ ID NO: 50 - RNA, 462 nt, Homo sapiens
  source 1..462: mol_type = other RNA
ggcacgtagtaaaaaccaagtggagcagagtcctcagtccctgatcatcctggagggaaagaactgcactcttcaatgcaattatacagtgagccccttcagcaacttaaggtggtataagcaagatactgggagaggtcctgtttccctgacaatcatgactttcagtgagaacacaaagtcgaacggaagatatacagcaactctggatgcagacacaaagcaaagctctctgcacatcacagcctcccagctcagcgattcagcctcctacatctgtgtggtgagcgagcagcgaccgggatgacaactgacagctgggggaaattgcagtttggagcagggacccaggttgtggtcaccccagcacgtagtatatccagaaccctgaccctgccgtgtaccagctgagagactctaaatccagtgacaagtctgtctgcctattcaccgattttgatt

SEQ ID NO: 51 - DNA, 570 bp, Homo sapiens
  source 1..570: mol_type = other DNA
caatccgccctcactacaaccggatacgccggtctcgaatgtaatacgactcactataggcacgtagtaatgctggtgtcactcagaccccaaaattccacgtcctgaagacaggacagagcatgactctgctgtgtgcccaggatatgaaccatgaatacatgtatcggtatcgacaagacccaggcaaggggctgaggctgatttactactcagttgctgctgctctcactgacaaaggagaagttcccaatggctacaatgtctccagatcaaacacagaggatttccccctcaagctggagtcagctgctccctctcagacttctgtttacttctgtgccagcagttactcagcgggaggccgggggacagggggcaaccagccaaaaacattcagtacttcggcgccgggacccggctctcagtgctggcacgtagtgaggacctgaacaaggtgttcccacccgaggtcgctgtgtttgagccatcagaagcagagatctcccacacccaaaaggccacactggtgattcggagacctcccgctactctggcgtcgatgaggga

SEQ ID NO: 52 - RNA, 474 nt, Homo sapiens
  source 1..474: mol_type = other RNA
ggcacgtagtaatgctggtgtcactcagaccccaaaattccacgtcctgaagacaggacagagcatgactctgctgtgtgcccaggatatgaaccatgaatacatgtatcggtatcgacaagacccaggcaaggggctgaggctgatttactactcagttgctgctgctctcactgacaaaggagaagttcccaatggctacaatgtctccagatcaaacacagaggatttccccctcaagctggagtcagctgctccctctcagacttctgtttacttctgtgccagcagttactcagcgggaggccgggggacagggggcaaccagccaaaaacattcagtacttcggcgccgggacccggctctcagtgctggcacgtagtgaggacctgaacaaggtgttcccacccgaggtcgctgtgtttgagccatcagaagcagagatctcccacacccaaaaggccacactggtg

SEQ ID NO: 53 - DNA, 567 bp, Homo sapiens
  source 1..567: mol_type = other DNA
caatccgccctcactacaaccggatacgccggtctcgaatgtaatacgactcactataggcacgtagtgacacagctgtttcccagactccaaaatacctggtcacacagatgggaaacgacaagtccattaaatgtgaacaaaatctgggccatgatactatgtattggtataaacaggactctaagaaatttctgaagataatgtttagctacaataataaggagctcattataaatgaaacagttccaaatcgcttctcacctaaatctccagacaaagctcacttaaatcttcacatcaattccctggagcttggtgactctgctgtgtatttctgtgccagcagccaagagaaggacaggggacagggggcgaaacgacctcctacgagcagtacttcgggccgggcaccaggctcacggtcacagcacgtagtgaggacctgaacaaggtgttcccacccgaggtcgctgtgtttgagccatcagaagcagagatctcccacacccaaaaggccacactggtgattcggagacctcccgctactctggcgtcgatgaggga

SEQ ID NO: 54 - RNA, 471 nt, Homo sapiens
  source 1..471: mol_type = other RNA
ggcacgtagtgacacagctgtttcccagactccaaaatacctggtcacacagatgggaaacgacaagtccattaaatgtgaacaaaatctgggccatgatactatgtattggtataaacaggactctaagaaatttctgaagataatgtttagctacaataataaggagctcattataaatgaaacagttccaaatcgcttctcacctaaatctccagacaaagctcacttaaatcttcacatcaattccctggagcttggtgactctgctgtgtatttctgtgccagcagccaagagaaggacaggggacagggggcgaaacgacctcctacgagcagtacttcgggccgggcaccaggctcacggtcacagcacgtagtgaggacctgaacaaggtgttcccacccgaggtcgctgtgtttgagccatcagaagcagagatctcccacacccaaaaggccacactggtg

SEQ ID NO: 55 - DNA, 577 bp, Homo sapiens
  source 1..577: mol_type = other DNA
caatccgccctcactacaaccggatacgccggtctcgaatgtaatacgactcactataggcacgtagttgtgacaaagtaacccagagttccccggaccagacggtggcgagtggcagtgaggtggtactgctctgcacttacgacactgtatattcaaatccagatttattctggtactggataaggccagattattcctttcagtttgtcttttatggggataacagcagatcagaaggtgcagattttactcaaggacggttttctgtgaaacacattctgacccagaaagcctttcacttggtgatctctccagtaaggactgaagacagtgccacttactactgtgcctttagacaaggcccaaggacgactgggggatacgaaaacacgccagacccctgatctttggcaaaggaacctatctggaggtacaacaaccacgtagtgaagtcagcctcataccaaaccatccgtttttgtcatgaaaaatggaacaaatgtcgcttgtctggtgaaggaattctaccccaaggaattcggagacctcccgctactctggcgtcgatgaggga

SEQ ID NO: 56 - RNA, 481 nt, Homo sapiens
  source 1..481: mol_type = other RNA
ggcacgtagttgtgacaaagtaacccagagttccccggaccagacggtggcgagtggcagtgaggtggtactgctctgcacttacgacactgtatattcaaatccagatttattctggtactggataaggccagattattcctttcagtttgtcttttatggggataacagcagatcagaaggtgcagattttactcaaggacggttttctgtgaaacacattctgacccagaaagcctttcacttggtgatctctccagtaaggactgaagacagtgccacttactactgtgcctttagacaaggcccaaggacgactgggggatacgaaaacacgccagacccctgatctttggcaaaggaacctatctggaggtacaacaaccacgtagtgaagtcagcctcataccaaaccatccgtttttgtcatgaaaaatggaacaaatgtcgcttgtctggtgaaggaattctaccccaagga

SEQ ID NO: 57 - DNA, 575 bp, Homo sapiens
  source 1..575: mol_type = other DNA
caatccgccctcactacaaccggatacgccggtctcgaatgtaatacgactcactataggcacgtagtattgagttggtgcctgaacaccaaacagtgcctgtgtcaatagggatccctgccaccctcaggtgctccatgaaaggagaagcgatcggtaactactatatcaactggtacaggaagacccaaggtaacacaatcactttcatataccgagaaaaggacatctatggccctggtttcaaagacaatttccaaggtgacattgatattgcaaagaacctggctgtacttaagatacttgcaccatcagagagagatgaagggtcttactactgtgcctgtgacacccaacgagacgccgccttcctaccggacagaaccgctttgacagcacaactcttctttggaaagggaacacaactcatcgtggaaccagcacgtagtgaagtcagcctcataccaaaccatccgtttttgtcatgaaaaatggaacaaatgtcgcttgtctggtgaaggaattctaccccaaggaattcggagacctcccgctactctggcgtcgatgaggga

SEQ ID NO: 58 - RNA, 479 nt, Homo sapiens
  source 1..479: mol_type = other RNA
ggcacgtagtattgagttggtgcctgaacaccaaacagtgcctgtgtcaatagggatccctgccaccctcaggtgctccatgaaaggagaagcgatcggtaactactatatcaactggtacaggaagacccaaggtaacacaatcactttcatataccgagaaaaggacatctatggccctggtttcaaagacaatttccaaggtgacattgatattgcaaagaacctggctgtacttaagatacttgcaccatcagagagagatgaagggtcttactactgtgcctgtgacacccaacgagacgccgccttcctaccggacagaaccgctttgacagcacaactcttctttggaaagggaacacaactcatcgtggaaccagcacgtagtgaagtcagcctcataccaaaccatccgtttttgtcatgaaaaatggaacaaatgtcgcttgtctggtgaaggaattctaccccaagga

SEQ ID NO: 59 - DNA, 584 bp, Homo sapiens
  source 1..584: mol_type = other DNA
caatccgccctcactacaaccggatacgccggtctcgaatgtaatacgactcactataggcacgtagttcttccaacttggaagggagaacgaagtcagtcaccaggcagactgggtcatctgctgaaatcacttgcgatcttactgtaacaaataccttctacatccactggtacctacaccaggaggggaaggccccacagcgtcttctgtactatgacgtctccactgcaagggatgtgttggaatcaggactcagtccaggaaagtattatactcatacacccaggaggtggagctggatattgagactgcaaaatctaattgaaaatgattctggggtctattactgtgccacctgggacagcgacgcagaacccggaccagcataccactggttggttcaagatatttgctgaagggactaagctcatagtaacttcacctgcacgtagtgataaacaacttgatgcagatgtttcccccaagcccactatttttcttccttcgattgctgaaacaaaactccagaaggctggaacatacattcggagacctcccgctactctggcgtcgatgaggga

SEQ ID NO: 60 - RNA, 488 nt, Homo sapiens
  source 1..488: mol_type = other RNA
ggcacgtagttcttccaacttggaagggagaacgaagtcagtcaccaggcagactgggtcatctgctgaaatcacttgcgatcttactgtaacaaataccttctacatccactggtacctacaccaggaggggaaggccccacagcgtcttctgtactatgacgtctccactgcaagggatgtgttggaatcaggactcagtccaggaaagtattatactcatacacccaggaggtggagctggatattgagactgcaaaatctaattgaaaatgattctggggtctattactgtgccacctgggacagcgacgcagaacccggaccagcataccactggttggttcaagatatttgctgaagggactaagctcatagtaacttcacctgcacgtagtgataaacaacttgatgcagatgtttcccccaagcccactatttttcttccttcgattgctgaaacaaaactccagaaggctggaacatac

SEQ ID NO: 61 - DNA, 563 bp, Homo sapiens
  source 1..563: mol_type = other DNA
caatccgccctcactacaaccggatacgccggtctcgaatgtaatacgactcactataggcacgtagttcttccaacttggaagggagaacgaagtcagtcaccaggctgactgggtcatctgctgaaatcacctgtgatcttcctggagcaagtaccttatacatccactggtacctgcaccaggaggggaaggccccacagtgtcttctgtactatgaaccctactactccagggttgtgctggaatcaggaatcactccaggaaagtatgacactggaagcacaaggagcaattggaatttgagactgcaaaatctaattaaaaatgattctgggttctattactgtgccacctgggacaggagacgcaccgaagaattattataagaaactctttggcagtggaacaacactggttgtcacagcacgtagtgataaacaacttgatgcagatgtttcccccaagcccactatttttcttccttcgattgctgaaacaaaactccagaaggctggaacatacattcggagacctcccgctactctggcgtcgatgaggga

SEQ ID NO: 62 - RNA, 467 nt, Homo sapiens
  source 1..467: mol_type = other RNA
ggcacgtagttcttccaacttggaagggagaacgaagtcagtcaccaggctgactgggtcatctgctgaaatcacctgtgatcttcctggagcaagtaccttatacatccactggtacctgcaccaggaggggaaggccccacagtgtcttctgtactatgaaccctactactccagggttgtgctggaatcaggaatcactccaggaaagtatgacactggaagcacaaggagcaattggaatttgagactgcaaaatctaattaaaaatgattctgggttctattactgtgccacctgggacaggagacgcaccgaagaattattataagaaactctttggcagtggaacaacactggttgtcacagcacgtagtgataaacaacttgatgcagatgtttcccccaagcccactatttttcttccttcgattgctgaaacaaaactccagaaggctggaacatac

SEQ ID NO: 63 - DNA, 607 bp, Homo sapiens
  source 1..607: mol_type = other DNA
caatccgccctcactacaaccggatacgccggtctcgaatgtaatacgactcactataggcacgtagtagagagcgtgtcattatgtgccggcacaggagtctagagtccgacgagtcgaggtacatccgacacgagcacctaagcaggcgccattagcactgagacgggaccttgaagacacgcatcaaacgacatggtttctatccctactagggagggtaggtgagttcgggaacaggtccccggacagcgtgggtcgactatcgtatcgacgacttccacggaggtcttcggaacgtcctctggaagtggctcctgggtccgaagaagtggagtcggggtctgacgtggtcgacctggacacaaccacacggcatcaatgacatcagtgggagggactcctctgccactggcaccagggaacgggggtctgcaggtatggcatcatcatcatcattacacgtagtgggagtgcatccgccccaacccttttccccctcgtctcctgtgagaattccccgtcggatacgagcagcgtggccgttggctgcctcgcacaggacttccattcggagacctcccgctactctggcgtcgatgaggga

SEQ ID NO: 64 - RNA, 511 nt, Homo sapiens
  source 1..511: mol_type = other RNA
ggcacgtagtagagagcgtgtcattatgtgccggcacaggagtctagagtccgacgagtcgaggtacatccgacacgagcacctaagcaggcgccattagcactgagacgggaccttgaagacacgcatcaaacgacatggtttctatccctactagggagggtaggtgagttcgggaacaggtccccggacagcgtgggtcgactatcgtatcgacgacttccacggaggtcttcggaacgtcctctggaagtggctcctgggtccgaagaagtggagtcggggtctgacgtggtcgacctggacacaaccacacggcatcaatgacatcagtgggagggactcctctgccactggcaccagggaacgggggtctgcaggtatggcatcatcatcatcattacacgtagtgggagtgcatccgccccaacccttttccccctcgtctcctgtgagaattccccgtcggatacgagcagcgtggccgttggctgcctcgcacaggacttcc

SEQ ID NO: 65 - DNA, 560 bp, Homo sapiens
  source 1..560: mol_type = other DNA
caatccgccctcactacaaccggatacgccggtctcgaatgtaatacgactcactataggcacgtagtcacaggcgccgccagtgtctcgagtcgaagtccctcttgaccaagaacctgcacagatgcctataccactgagctgagaactccctgcccaacatcatccacgagggtgatattatctatgagggttaggtgaggtcggggaagggacccccgaccgcctaggtcggggtcatcattgatgatgacgactacctcgcggcgaacccagacggaaccacagaaacagagagagagaagaacaaggagaaagaggaagaggaagaagaagagaagaggaggagaaggcagaaccgaaagggactcctctgccactggtcccaaggaaccggggtcatcagtttcatcacacgtagtgggagtgcatccgccccaacccttttccccctcgtctcctgtgagaattccccgtcggatacgagcagcgtggccgttggctgcctcgcacaggacttccattcggagacctcccgctactctggcgtcgatgaggga

SEQ ID NO: 66 - RNA, 464 nt, Homo sapiens
  source 1..464: mol_type = other RNA
ggcacgtagtcacaggcgccgccagtgtctcgagtcgaagtccctcttgaccaagaacctgcacagatgcctataccactgagctgagaactccctgcccaacatcatccacgagggtgatattatctatgagggttaggtgaggtcggggaagggacccccgaccgcctaggtcggggtcatcattgatgatgacgactacctcgcggcgaacccagacggaaccacagaaacagagagagagaagaacaaggagaaagaggaagaggaagaagaagagaagaggaggagaaggcagaaccgaaagggactcctctgccactggtcccaaggaaccggggtcatcagtttcatcacacgtagtgggagtgcatccgccccaacccttttccccctcgtctcctgtgagaattccccgtcggatacgagcagcgtggccgttggctgcctcgcacaggacttcc

SEQ ID NO: 67 - DNA, 606 bp, Homo sapiens
  source 1..606: mol_type = other DNA
caatccgccctcactacaaccggatacgccggtctcgaatgtaatacgactcactataggcacgtagtggagagcgtgtcattatgtgtcggcacaggcgccgccagtgtctcgagtcgaagtccctcttgaccaagaacctgcacagatgactataccaccgagctgagaactccctgcccaacaacaaccacgaaggtgatactaactaaaggggttaggtgaggtcggggaagggatccccgaccgcctaggtcgtggtcatcattggtgacttcctgggtggtatctgtcgcgtccactccctgtcccagaggcttccgaagttgtcaggacgcggggtgacgacatcgacgtggacgcccgaggaaacagcattagcatctgtgatataggtgagcggaagaccacagactcctctgccactggtcccagggaaccggggtcatcagtttcatcgcacgtagtgggagtgcatccgccccaacccttttccccctcgtctcctgtgagaattccccgtcggatacgagcagcgtggccgttggctgcctcgcacaggacttccattcggagacctcccgctactctggcgtcgatgaggga

SEQ ID NO: 68 - RNA, 510 nt, Homo sapiens
  source 1..510: mol_type = other RNA
ggcacgtagtggagagcgtgtcattatgtgtcggcacaggcgccgccagtgtctcgagtcgaagtccctcttgaccaagaacctgcacagatgactataccaccgagctgagaactccctgcccaacaacaaccacgaaggtgatactaactaaaggggttaggtgaggtcggggaagggatccccgaccgcctaggtcgtggtcatcattggtgacttcctgggtggtatctgtcgcgtccactccctgtcccagaggcttccgaagttgtcaggacgcggggtgacgacatcgacgtggacgcccgaggaaacagcattagcatctgtgatataggtgagcggaagaccacagactcctctgccactggtcccagggaaccggggtcatcagtttcatcgcacgtagtgggagtgcatccgccccaacccttttccccctcgtctcctgtgagaattccccgtcggatacgagcagcgtggccgttggctgcctcgcacaggacttcc

SEQ ID NO: 69 - DNA, 648 bp, Homo sapiens
  source 1..648: mol_type = other DNA
caatccgccctcactacaaccggatacgccggtctcgaatgtaatacgactcactataggcacgtagtggagagcgtgtcattatgtgtcggcacaggcgccgccagtgtctcgagtcgaagtccctcttgaccaagaacctgcacagatgactataccactgagctgagaactccctgcccaacatcaaccacgaaggtgatactaactaaaggggttaggtgaggtcggggaagggacccccgaccgcctaggtcgaggtcatcattggtgacttcctgggtggtatctgtcgcgtccactccctgtcccagaggcttccgaagttgtcaggacgcggggtgacaacatcgacgtggaccgcccgcagcgcagaccacagaaacagagagagagaagaacaaggagaaagaggaagaggaagaagaagagaagaggaggagaagggcagcaagactcctctgccactggtcccagggaaccggggtcatcagtttcatcgcacgtagtgcacccaccaaggctccggatgtgttccccatcatatcagggtgcagacacccaaaggataacagccctgtggtcctggcatgcttgataactgggtaccattcggagacctcccgctactctggcgtcgatgaggga

SEQ ID NO: 70 - RNA, 552 nt, Homo sapiens
  source 1..552: mol_type = other RNA
ggcacgtagtggagagcgtgtcattatgtgtcggcacaggcgccgccagtgtctcgagtcgaagtccctcttgaccaagaacctgcacagatgactataccactgagctgagaactccctgcccaacatcaaccacgaaggtgatactaactaaaggggttaggtgaggtcggggaagggacccccgaccgcctaggtcgaggtcatcattggtgacttcctgggtggtatctgtcgcgtccactccctgtcccagaggcttccgaagttgtcaggacgcggggtgacaacatcgacgtggaccgcccgcagcgcagaccacagaaacagagagagagaagaacaaggagaaagaggaagaggaagaagaagagaagaggaggagaagggcagcaagactcctctgccactggtcccagggaaccggggtcatcagtttcatcgcacgtagtgcacccaccaaggctccggatgtgttccccatcatatcagggtgcagacacccaaaggataacagccctgtggtcctggcatgcttgataactgggtacc

SEQ ID NO: 71 - DNA, 586 bp, Homo sapiens
  source 1..586: mol_type = other DNA
caatccgccctcactacaaccggatacgccggtctcgaatgtaatacgactcactataggcacgtagtagagagcgtgtcgttatgtgccggcacaggcgccgccagtgtctcgagtccaagtccctcttgaccaagaacctgcacagatgactataccaccgagctgagaactccctccccaacaacaaccacgagggtgatattatctatatgggttaggtgaggtcggggaagggacccccgacggcctaggtcgaggtcatcattggtgactgcctgggtggtatctgtcgcgtccactccctgtcccagaggcttccgaagttgtcaggacgcggggtgacgacatcgacgtggacggacggccggcagcaaggtcaatatggacgggactcctctgccactggtcccaagggaccggggtcatcagtttcatcacacgtagtgcacccaccaaggctccggatgtgttccccatcatatcagggtgcagacacccaaaggataacagccctgtggtcctggcatgcttgataactgggtaccattcggagacctcccgctactctggcgtcgatgaggga

SEQ ID NO: 72 - RNA, 490 nt, Homo sapiens
  source 1..490: mol_type = other RNA
ggcacgtagtagagagcgtgtcgttatgtgccggcacaggcgccgccagtgtctcgagtccaagtccctcttgaccaagaacctgcacagatgactataccaccgagctgagaactccctccccaacaacaaccacgagggtgatattatctatatgggttaggtgaggtcggggaagggacccccgacggcctaggtcgaggtcatcattggtgactgcctgggtggtatctgtcgcgtccactccctgtcccagaggcttccgaagttgtcaggacgcggggtgacgacatcgacgtggacggacggccggcagcaaggtcaatatggacgggactcctctgccactggtcccaagggaccggggtcatcagtttcatcacacgtagtgcacccaccaaggctccggatgtgttccccatcatatcagggtgcagacacccaaaggataacagccctgtggtcctggcatgcttgataactgggtacc

SEQ ID NO: 73 - DNA, 552 bp, Homo sapiens
  source 1..552: mol_type = other DNA
caatccgccctcactacaaccggatacgccggtctcgaatgtaatacgactcactataggcacgtagttttaggacaggtgagttcgggaacaggtccctgggtagcgtgggtcacgttcatcgtcatccacttccacataggtcttcggaacgtcctctggaagtgactccggggtccgaagaagtggagtcggggtctgacgtggtcgacgtggtcacgggcgacacgcacacaatgcccaccaccgaccccgacaccaacggaaccgctccgacgggagacgaaccccaacacgtcacccgtgactcctccggacacggaaacctctgagccacgccggagactcctctgccactggcaccagggaaccggggtctgcaggtatggcatcatcatcatcattacacgtagtgcacccaccaaggctccggatgtgttccccatcatatcagggtgcagacacccaaaggataacagccctgtggtcctggcatgcttgataactgggtaccattcggagacctcccgctactctggcgtcgatgaggga

SEQ ID NO: 74 - RNA, 456 nt, Homo sapiens
  source 1..456: mol_type = other RNA
ggcacgtagttttaggacaggtgagttcgggaacaggtccctgggtagcgtgggtcacgttcatcgtcatccacttccacataggtcttcggaacgtcctctggaagtgactccggggtccgaagaagtggagtcggggtctgacgtggtcgacgtggtcacgggcgacacgcacacaatgcccaccaccgaccccgacaccaacggaaccgctccgacgggagacgaaccccaacacgtcacccgtgactcctccggacacggaaacctctgagccacgccggagactcctctgccactggcaccagggaaccggggtctgcaggtatggcatcatcatcatcattacacgtagtgcacccaccaaggctccggatgtgttccccatcatatcagggtgcagacacccaaaggataacagccctgtggtcctggcatgcttgataactgggtacc

SEQ ID NO: 75 - DNA, 648 bp, Homo sapiens
  source 1..648: mol_type = other DNA
caatccgccctcactacaaccggatacgccggtctcgaatgtaatacgactcactataggcacgtagtagagcgtgtcattatgtaccgccacaggctccggaagtccgacgaggtgacgtccatccgccacgactacccgaacagccgactctaccactggaccggaaccttcctgcccgacatagaccatagtctcagtggtcctatctactaggggtaggtgaggtccggaaagggcccgtagaccgcgtgggtcggctaggtcatcgaccatttcgacataggtcttgggaatgtcctctagaagtctctgagggggccgaaaaagtggagacgaggtctgacgtggtcgacgtggagggggccaggccaccacagaaacagagagagagaagaacaaggagaaagaggaagaggaagaagaagagaagaggaggagaagggcgaaagcagactcctctgccactggtcccaagggaccggggtcatcagtttcatcacacgtagtgcatccccgaccagccccaaggtcttcccgctgagcctctgcagcaccccccaagatgggaacgtggtcgtcgcatgcctggtccagggcttcttcccccattcggagacctcccgctactctggcgtcgatgaggga

SEQ ID NO: 76 - RNA, 552 nt, Homo sapiens
  source 1..552: mol_type = other RNA
ggcacgtagtagagcgtgtcattatgtaccgccacaggctccggaagtccgacgaggtgacgtccatccgccacgactacccgaacagccgactctaccactggaccggaaccttcctgcccgacatagaccatagtctcagtggtcctatctactaggggtaggtgaggtccggaaagggcccgtagaccgcgtgggtcggctaggtcatcgaccatttcgacataggtcttgggaatgtcctctagaagtctctgagggggccgaaaaagtggagacgaggtctgacgtggtcgacgtggagggggccaggccaccacagaaacagagagagagaagaacaaggagaaagaggaagaggaagaagaagagaagaggaggagaagggcgaaagcagactcctctgccactggtcccaagggaccggggtcatcagtttcatcacacgtagtgcatccccgaccagccccaaggtcttcccgctgagcctctgcagcaccccccaagatgggaacgtggtcgtcgcatgcctggtccagggcttcttccccc

SEQ ID NO: 77 - DNA, 623 bp, Homo sapiens
  source 1..623: mol_type = other DNA
caatccgccctcactacaaccggatacgccggtctcgaatgtaatacgactcactataggcacgtagtagagcgtgtcattatgtaccgccacaggctccggaagtccgacgaggtgacgtccatccgccacgactacctgaacagccgactctaccattggaccggaaccttcctgcccgacatagaccatagtctcagtggtcctatctactaggggtaggtgaggtccggaaagggcccgtagaccgcgtgggtcggctaggtcatcgaccatttcgacataggtcttgggaatgtcctctagaagtctctgagggggccgaaaaagtggagacgaggtctgacgtggtcgacgtggagcgccccagcaagcaatattattgaggggcttgtatcattatggcggggcagagactcctctgccactggcaccagggaaccggggtctgcaggtatggcatcatcatcatcattacacgtagtgcatccccgaccagccccaaggtcttcccgctgagcctctgcagcaccccccaagatgggaacgtggtcgtcgcatgcctggtccagggcttcttcccccattcggagacctcccgctactctggcgtcgatgaggga

SEQ ID NO: 78 - RNA, 527 nt, Homo sapiens
  source 1..527: mol_type = other RNA
ggcacgtagtagagcgtgtcattatgtaccgccacaggctccggaagtccgacgaggtgacgtccatccgccacgactacctgaacagccgactctaccattggaccggaaccttcctgcccgacatagaccatagtctcagtggtcctatctactaggggtaggtgaggtccggaaagggcccgtagaccgcgtgggtcggctaggtcatcgaccatttcgacataggtcttgggaatgtcctctagaagtctctgagggggccgaaaaagtggagacgaggtctgacgtggtcgacgtggagcgccccagcaagcaatattattgaggggcttgtatcattatggcggggcagagactcctctgccactggcaccagggaaccggggtctgcaggtatggcatcatcatcatcattacacgtagtgcatccccgaccagccccaaggtcttcccgctgagcctctgcagcaccccccaagatgggaacgtggtcgtcgcatgcctggtccagggcttcttccccc

SEQ ID NO: 79 - DNA, 593 bp, Homo sapiens
  source 1..593: mol_type = other DNA
caatccgccctcactacaaccggatacgccggtctcgaatgtaatacgactcactataggcacgtagtagagagcgtgtcattatgtgccggcacaggagtctagagtccgacgagtcgaggtacatccgacacgagcacctaaacaggcgccattagcactgagacgggaccttgaagacacgcatcaaacgacatggtttctatccctactagggagggtaggtgagttcgggaacaggtccccggacagcgtgggtcgactatcgtatcgacgacttccacggaggtcttcggaacgtcctctggaagtggctcctgggtccgaagaagtggagtcggggtctgacgtggtcgacctggacagcaacgacgcacatcaaggtcaatatggaacagacttctctgccactggtaacagggaaccggggtctgtagttttcgtagtcacgtagtgcatccccgaccagccccaaggtcttcccgctgagcctctgcagcaccccccaagatgggaacgtggtcgtcgcatgcctggtccagggcttcttcccccattcggagacctcccgctactctggcgtcgatgaggga

SEQ ID NO: 80 - RNA, 497 nt, Homo sapiens
  source 1..497: mol_type = other RNA
ggcacgtagtagagagcgtgtcattatgtgccggcacaggagtctagagtccgacgagtcgaggtacatccgacacgagcacctaaacaggcgccattagcactgagacgggaccttgaagacacgcatcaaacgacatggtttctatccctactagggagggtaggtgagttcgggaacaggtccccggacagcgtgggtcgactatcgtatcgacgacttccacggaggtcttcggaacgtcctctggaagtggctcctgggtccgaagaagtggagtcggggtctgacgtggtcgacctggacagcaacgacgcacatcaaggtcaatatggaacagacttctctgccactggtaacagggaaccggggtctgtagttttcgtagtcacgtagtgcatccccgaccagccccaaggtcttcccgctgagcctctgcagcaccccccaagatgggaacgtggtcgtcgcatgcctggtccagggcttcttccccc

SEQ ID NO: 81 - DNA, 589 bp, Homo sapiens
  source 1..589: mol_type = other DNA
caatccgccctcactacaaccggatacgccggtctcgaatgtaatacgactcactataggcacgtagtgtcattatgtgccggcacaggcgccgccagtgtctcgggtcgaagtccctcttgaccaagaacctgcacagatgactataccactgagctgagaactccctgcccaacatcaaccacgaaggtgatactaactaaaggggttaggtgaggtcggggaagggacccccgaccgcctaggtcgaggtcatcattggtgacttcctgggtggtatctgtcgcgtccactccctgtcccagaggcttccgaagttgtcaggacgcggggtgacgacatcgacgtggacagaggcagcgacgccatcaatgacatcagtagagcacggaagacttctctgccactggtaacagggaaccggggtctgtagttttcgtagtcacgtagtgcctccacacagagcccatccgtcttccccttgacccgctgctgcaaaaacattccctccaatgccacctccgtgactctgggctgcctggccacgggctattcggagacctcccgctactctggcgtcgatgaggga

SEQ ID NO: 82 - RNA, 493 nt, Homo sapiens
  source 1..493: mol_type = other RNA
ggcacgtagtgtcattatgtgccggcacaggcgccgccagtgtctcgggtcgaagtccctcttgaccaagaacctgcacagatgactataccactgagctgagaactccctgcccaacatcaaccacgaaggtgatactaactaaaggggttaggtgaggtcggggaagggacccccgaccgcctaggtcgaggtcatcattggtgacttcctgggtggtatctgtcgcgtccactccctgtcccagaggcttccgaagttgtcaggacgcggggtgacgacatcgacgtggacagaggcagcgacgccatcaatgacatcagtagagcacggaagacttctctgccactggtaacagggaaccggggtctgtagttttcgtagtcacgtagtgcctccacacagagcccatccgtcttccccttgacccgctgctgcaaaaacattccctccaatgccacctccgtgactctgggctgcctggccacgggct

SEQ ID NO: 83 - DNA, 650 bp, Homo sapiens
  source 1..650: mol_type = other DNA
caatccgccctcactacaaccggatacgccggtctcgaatgtaatacgactcactataggcacgtagtgagagcgtgtcattatgtgtcggcacagaagtctagagtccgacgagtcgaggtacatccgacacgagcgcctacacagggaccattaccactgagacgggaccttgaagacacttataaaacacaatggtaacggtcacaactaggtagggtaggtgagttcggaaacaggcccccggaccgcgtgggttacgtatcgtatcgatcacttccacataggtcttcggaacgtcctttggaagtgactccggggtccgaagaagtggagtcggggtctgacgtgttcgacctggacaacgacaacaaaaacaccacagaaacagagagagagaagaacaaggagaaagaggaagaggaagaagaagagaagaggaggagaaggcaagcagactcctctgccactggtcccaagggaccggggtcatcagtttcatcacacgtagtgcctccacacagagcccatccgtcttccccttgacccgctgctgcaaaaacattccctccaatgccacctccgtgactctgggctgcctggccacgggctattcggagacctcccgctactctggcgtcgatgaggga

SEQ ID NO: 84 - RNA, 554 nt, Homo sapiens
  source 1..554: mol_type = other RNA
ggcacgtagtgagagcgtgtcattatgtgtcggcacagaagtctagagtccgacgagtcgaggtacatccgacacgagcgcctacacagggaccattaccactgagacgggaccttgaagacacttataaaacacaatggtaacggtcacaactaggtagggtaggtgagttcggaaacaggcccccggaccgcgtgggttacgtatcgtatcgatcacttccacataggtcttcggaacgtcctttggaagtgactccggggtccgaagaagtggagtcggggtctgacgtgttcgacctggacaacgacaacaaaaacaccacagaaacagagagagagaagaacaaggagaaagaggaagaggaagaagaagagaagaggaggagaaggcaagcagactcctctgccactggtcccaagggaccggggtcatcagtttcatcacacgtagtgcctccacacagagcccatccgtcttccccttgacccgctgctgcaaaaacattccctccaatgccacctccgtgactctgggctgcctggccacgggct

SEQ ID NO: 85 - DNA, 610 bp, Homo sapiens
  source 1..610: mol_type = other DNA
caatccgccctcactacaaccggatacgccggtctcgaatgtaatacgactcactataggcacgtagtagagagcgtgtcattatgtgccggcacaggagtctagagtccgacgagtcgaggtacatccgacacgagcacctaagcaggcgccattagcactgagacgggaccttgaagacacgcatcaaacgacatggttcctatccctactaggaagggtaggtgagttcgggaacaggtccccggacagcgtgggtcgactatcgtatcgacgacttccacggaggtcttcggaacgtcctctggaagtggctcctgggtccgaagaagtggagtcggggtctgacgtggtcgacctggacaggagcggccgccatcaatcgtggtatcagtaaccaagactcctctgccactggcaccagggaaccggggtctgcaggtatggcatcatcatcatcattacacgtagtgcctccacacagagcccatccgtcttccccttgacccgctgctgcaaaaacattccctccaatgccacctccgtgactctgggctgcctggccacgggctattcggagacctcccgctactctggcgtcgatgaggga

SEQ ID NO: 86 - RNA, 514 nt, Homo sapiens
  source 1..514: mol_type = other RNA
ggcacgtagtagagagcgtgtcattatgtgccggcacaggagtctagagtccgacgagtcgaggtacatccgacacgagcacctaagcaggcgccattagcactgagacgggaccttgaagacacgcatcaaacgacatggttcctatccctactaggaagggtaggtgagttcgggaacaggtccccggacagcgtgggtcgactatcgtatcgacgacttccacggaggtcttcggaacgtcctctggaagtggctcctgggtccgaagaagtggagtcggggtctgacgtggtcgacctggacaggagcggccgccatcaatcgtggtatcagtaaccaagactcctctgccactggcaccagggaaccggggtctgcaggtatggcatcatcatcatcattacacgtagtgcctccacacagagcccatccgtcttccccttgacccgctgctgcaaaaacattccctccaatgccacctccgtgactctgggctgcctggccacgggct

SEQ ID NO: 87 - DNA, 600 bp, Homo sapiens
  source 1..600: mol_type = other DNA
caatccgccctcactacaaccggatacgccggtctcgaatgtaatacgactcactataggcacgtagtagagagcgtgtcattatgtgccggcacaggagtctagagtccgacgagtcgaggtacatccgacacgagcacctaaacaggcgccattagcactgagacgggaccttgaagacacgcatcaaacgatatggtttctatccctactaggaagggtaggtgagttcgggaacaggtccccggacagcgtgggtcgactatcgtatcgacgacttccacggaggtcttcggaacgtcctctggaagtggctcctgggtccgaagaagtggagtcggggtctgacgtggtcgacctggacggcgacgccgtatcgtcgaccatgatgatgttataggaggccgactcctctgccactggtcccaaggaaccggggtcatcagtttcatcacacgtagtgcctccaccaagggcccatcggtcttccccctggtgccctcctccaggagcgtctctgagggcacagcggccctgggctgcctggtcaaggactacttccattcggagacctcccgctactctggcgtcgatgaggga

SEQ ID NO: 88 - RNA, 504 nt, Homo sapiens
  source 1..504: mol_type = other RNA
ggcacgtagtagagagcgtgtcattatgtgccggcacaggagtctagagtccgacgagtcgaggtacatccgacacgagcacctaaacaggcgccattagcactgagacgggaccttgaagacacgcatcaaacgatatggtttctatccctactaggaagggtaggtgagttcgggaacaggtccccggacagcgtgggtcgactatcgtatcgacgacttccacggaggtcttcggaacgtcctctggaagtggctcctgggtccgaagaagtggagtcggggtctgacgtggtcgacctggacggcgacgccgtatcgtcgaccatgatgatgttataggaggccgactcctctgccactggtcccaaggaaccggggtcatcagtttcatcacacgtagtgcctccaccaagggcccatcggtcttccccctggtgccctcctccaggagcgtctctgagggcacagcggccctgggctgcctggtcaaggactacttcc

SEQ ID NO: 89 - DNA, 649 bp, Homo sapiens
  source 1..649: mol_type = other DNA
caatccgccctcactacaaccggatacgccggtctcgaatgtaatacgactcactataggcacgtagtagagagcgtgtcattatgtgtcggtacaggagtctagagtccgacgagtcgaggtacatccgacacgagcgcctacacagggaccattaccactgagacgggaccttgaggacacttataaaacacaatggtaacggtcgcaactaggtagggtaggtgagttcggaaacaggcccccggaccgcgtgggttacgtatcgtatcgatcacttccacataggtcttcggaacgtcctttggaagtgactccggggtccgaagaagtggagtcggggtctgacgtggtcgacctggaccagcagcagccgaccacagaaacagagagagagaagaacaaggagaaagaggaagaggaagaagaagagaagaggaggagaaggagaaggacttctctgccactggtaacagggaaccggggtctatagttttcgtagtcacgtagtgcctccaccaagggcccatcggtcttccccctggtgccctcctccaggagcgtctctgagggcacagcggccctgggctgcctggtcaaggactacttccattcggagacctcccgctactctggcgtcgatgaggga

SEQ ID NO: 90 - RNA, 553 nt, Homo sapiens
  source 1..553: mol_type = other RNA
ggcacgtagtagagagcgtgtcattatgtgtcggtacaggagtctagagtccgacgagtcgaggtacatccgacacgagcgcctacacagggaccattaccactgagacgggaccttgaggacacttataaaacacaatggtaacggtcgcaactaggtagggtaggtgagttcggaaacaggcccccggaccgcgtgggttacgtatcgtatcgatcacttccacataggtcttcggaacgtcctttggaagtgactccggggtccgaagaagtggagtcggggtctgacgtggtcgacctggaccagcagcagccgaccacagaaacagagagagagaagaacaaggagaaagaggaagaggaagaagaagagaagaggaggagaaggagaaggacttctctgccactggtaacagggaaccggggtctatagttttcgtagtcacgtagtgcctccaccaagggcccatcggtcttccccctggtgccctcctccaggagcgtctctgagggcacagcggccctgggctgcctggtcaaggactacttcc

SEQ ID NO: 91 - DNA, 620 bp, Homo sapiens
  source 1..620: mol_type = other DNA
caatccgccctcactacaaccggatacgccggtctcgaatgtaatacgactcactataggcacgtagtagagagcgtgtcattatgtgccggcacaggagtctagagtccgacgagtcgaggtacatccgacacgagcacctaaacaggcgccattagcactgagacgggaccttgaagacacgcatcaaacgatatggtttctatccctactagggagggtaggtgagttcgggaacaggtccccggacagcgtgggtcgactatcgtatcgacgacttccacggaggtcttcggaacgtcctctggaagtggctcctgggtccgaagaagtggagtcggggtctgacgtggtcgacgtggaccgcccaacaagggcattagcatctgtgatataggtggcacggaaagagactcctctgccactggcaccagggaacgggggtctgcaggtatggcatcatcatcatcattacacgtagtgcctccaccaagggcccatcggtcttccccctggtgccctcctccaggagcgtctctgagggcacagcggccctgggctgcctggtcaaggactacttccattcggagacctcccgctactctggcgtcgatgaggga

SEQ ID NO: 92 - RNA, 524 nt, Homo sapiens
  source 1..524: mol_type = other RNA
ggcacgtagtagagagcgtgtcattatgtgccggcacaggagtctagagtccgacgagtcgaggtacatccgacacgagcacctaaacaggcgccattagcactgagacgggaccttgaagacacgcatcaaacgatatggtttctatccctactagggagggtaggtgagttcgggaacaggtccccggacagcgtgggtcgactatcgtatcgacgacttccacggaggtcttcggaacgtcctctggaagtggctcctgggtccgaagaagtggagtcggggtctgacgtggtcgacgtggaccgcccaacaagggcattagcatctgtgatataggtggcacggaaagagactcctctgccactggcaccagggaacgggggtctgcaggtatggcatcatcatcatcattacacgtagtgcctccaccaagggcccatcggtcttccccctggtgccctcctccaggagcgtctctgagggcacagcggccctgggctgcctggtcaaggactacttcc

SEQ ID NO: 93 - DNA, 546 bp, Homo sapiens
  source 1..546: mol_type = other DNA
caatccgccctcactacaaccggatacgccggtctcgaatgtaatacgactcactataggcacgtagtcctccattcaatactaggacgactgtcattatttgacgttttagaagtccgacgtccgacgactaccactctcacttcagagagggtctgggtgacggtgacttggaccgaccctacgatcaccgggaccacctacgtggtatctactcctcggacccgcggaccggtccaaagacgactatggtccaattcatcgacgacgattgtgagactgaccgggacgtcctctcccactgagaaaggggacctctgtttctgtcccacccacctctgacacagtaatgttaaagagaacacgccaacgcggagcaaagcaaactaaaggtggaaccagggaaccggcttgcaggtgcacgtagtcgaactgtggctgcaccatctgtcttcatcttcccgccatctgatgagcagttgaaatctggaactgcctctgttgtgtgattcggagacctcccgctactctggcgtcgatgaggga

SEQ ID NO: 94 - RNA, 450 nt, Homo sapiens
  source 1..450: mol_type = other RNA
ggcacgtagtcctccattcaatactaggacgactgtcattatttgacgttttagaagtccgacgtccgacgactaccactctcacttcagagagggtctgggtgacggtgacttggaccgaccctacgatcaccgggaccacctacgtggtatctactcctcggacccgcggaccggtccaaagacgactatggtccaattcatcgacgacgattgtgagactgaccgggacgtcctctcccactgagaaaggggacctctgtttctgtcccacccacctctgacacagtaatgttaaagagaacacgccaacgcggagcaaagcaaactaaaggtggaaccagggaaccggcttgcaggtgcacgtagtcgaactgtggctgcaccatctgtcttcatcttcccgccatctgatgagcagttgaaatctggaactgcctctgttgtgtg

SEQ ID NO: 95 - DNA, 538 bp, Homo sapiens
  source 1..538: mol_type = other DNA
caatccgccctcactacaaccggatacgccggtctcgaatgtaatacgactcactataggcacgtagtactccatttgatgatgagactactgtcattatgcaacgtcgtagaagtcgaaggtccgataactaccactcccactttagacagggtctaggtgacggtgacttggagctcccctggggactctacctgacccttcgtatgaactactcctcgaaacctctgactagaccaaagacgaccatggtcacattcgatgatggttacgagactgaccgggccgtccactaccactgaaagaggaaacctcagtgtctgactttcagacctctgactcagtcgtgttaaaggcaccaccgcagcaggcgcaaactagaggtcgaaccaggggaccggttttgacatgtcacgtagtcgaactgtggctgcaccatctgtcttcatcttcccgccatctgatgagcagttgaaatctggaactgcctctgttgtgtgattcggagacctcccgctactctggcgtcgatgaggga

SEQ ID NO: 96 - RNA, 442 nt, Homo sapiens
  source 1..442: mol_type = other RNA
ggcacgtagtactccatttgatgatgagactactgtcattatgcaacgtcgtagaagtcgaaggtccgataactaccactcccactttagacagggtctaggtgacggtgacttggagctcccctggggactctacctgacccttcgtatgaactactcctcgaaacctctgactagaccaaagacgaccatggtcacattcgatgatggttacgagactgaccgggccgtccactaccactgaaagaggaaacctcagtgtctgactttcagacctctgactcagtcgtgttaaaggcaccaccgcagcaggcgcaaactagaggtcgaaccaggggaccggttttgacatgtcacgtagtcgaactgtggctgcaccatctgtcttcatcttcccgccatctgatgagcagttgaaatctggaactgcctctgttgtgtg

SEQ ID NO: 97 - DNA, 533 bp, Homo sapiens
  source 1..533: mol_type = other DNA
caatccgccctcactacaaccggatacgccggtctcgaatgtaatacgactcactataggcacgtagtcctacggtcaacgatgcgacgactgtcattatgtgacgttttagaagtccgagatccgacgactaccactctcacttcagacagggtccgggtgacggtgacttggaccgaccctacggtcaccgggacaacctacgtagtatctactcctcggaccctcggaccggtccaaagacgaccatggtccgattcaacgacgattgtgggactgaccgggacgtcctctcccaccgagaaaggggacctctgtttctgtcccaccgacctctgacacagttgtgttaaaggacagccgcacgcccaaattagaggtcagcacagggaaccggcttccactagcacgtagtcgaactgtggctgcaccatctgtcttcatcttcccgccatctgatgagcagttgaaatctggaactgcctctgttgtgtgattcggagacctcccgctactctggcgtcgatgaggga

SEQ ID NO: 98 - RNA, 437 nt, Homo sapiens
  source 1..437: mol_type = other RNA
ggcacgtagtcctacggtcaacgatgcgacgactgtcattatgtgacgttttagaagtccgagatccgacgactaccactctcacttcagacagggtccgggtgacggtgacttggaccgaccctacggtcaccgggacaacctacgtagtatctactcctcggaccctcggaccggtccaaagacgaccatggtccgattcaacgacgattgtgggactgaccgggacgtcctctcccaccgagaaaggggacctctgtttctgtcccaccgacctctgacacagttgtgttaaaggacagccgcacgcccaaattagaggtcagcacagggaaccggcttccactagcacgtagtcgaactgtggctgcaccatctgtcttcatcttcccgccatctgatgagcagttgaaatctggaactgcctctgttgtgtg

SEQ ID NO: 99 - DNA, 557 bp, Homo sapiens
  source 1..557: mol_type = other DNA
caatccgccctcactacaaccggatacgccggtctcgaatgtaatacgactcactataggcacgtagtactcgtgactccgacgacagggtacgactcgtcattatcagtcggagcaggagtccgacctcaggtcattaccagtccctccgacacaaaggactggacctacgtctcttagagagactctagggactcccggccaacaataaggacatcctatcctcaaaccctcccaccgggaccacgacgacgtcggttcgacgaggaaccaacggttgttacaacgacaagggtcacgtccactcacaccgccagacagagttcgggaacctgtggctcccaccgactcagtcgggacggacgagcagaacacacagcaccgctcctgccagtcgacccacggaggaggcttgtgtcgtcacgtagtggtcagcccaaggctgccccctcggtcactctgttcccgccctcctctgaggagcttcaagccaacaaggccacactggtgtgtctcataattcggagacctcccgctactctggcgtcgatgaggga

SEQ ID NO: 100 - RNA, 461 nt, Homo sapiens
  source 1..461: mol_type = other RNA
ggcacgtagtactcgtgactccgacgacagggtacgactcgtcattatcagtcggagcaggagtccgacctcaggtcattaccagtccctccgacacaaaggactggacctacgtctcttagagagactctagggactcccggccaacaataaggacatcctatcctcaaaccctcccaccgggaccacgacgacgtcggttcgacgaggaaccaacggttgttacaacgacaagggtcacgtccactcacaccgccagacagagttcgggaacctgtggctcccaccgactcagtcgggacggacgagcagaacacacagcaccgctcctgccagtcgacccacggaggaggcttgtgtcgtcacgtagtggtcagcccaaggctgccccctcggtcactctgttcccgccctcctctgaggagcttcaagccaacaaggccacactggtgtgtctcata

SEQ ID NO: 101 - DNA, 557 bp, Homo sapiens
  source 1..557: mol_type = other DNA
caatccgccctcactacaaccggatacgccggtctcgaatgtaatacgactcactataggcacgtagtactcgtgactccgacgacagggtacgactcgtcattatcagtcggagcaggagtccgacctcaggtcattaccagtccctccgacacaaaggactggacctacgtctcttagagagactctagggactcccggccaacaataaggacatcctatcctcaaaccctcccaccgggaccacgacgacgtcggttcgacgaggaaccaacggtcgtaacaacgacaagggtcacgtccactcacaccgccagacagagttcgggaacctgtggctcccaccgactcagtcgggacggacggagaagccaggcgaagcgaattctgtcacccgacccagaacgacggtttgtacacgcacgtagtggtcagcccaaggctgccccctcggtcactctgttcccgccctcctctgaggagcttcaagccaacaaggccacactggtgtgtctcataattcggagacctcccgctactctggcgtcgatgaggga

SEQ ID NO: 102 - RNA, 461 nt, Homo sapiens
  source 1..461: mol_type = other RNA
ggcacgtagtactcgtgactccgacgacagggtacgactcgtcattatcagtcggagcaggagtccgacctcaggtcattaccagtccctccgacacaaaggactggacctacgtctcttagagagactctagggactcccggccaacaataaggacatcctatcctcaaaccctcccaccgggaccacgacgacgtcggttcgacgaggaaccaacggtcgtaacaacgacaagggtcacgtccactcacaccgccagacagagttcgggaacctgtggctcccaccgactcagtcgggacggacggagaagccaggcgaagcgaattctgtcacccgacccagaacgacggtttgtacacgcacgtagtggtcagcccaaggctgccccctcggtcactctgttcccgccctcctctgaggagcttcaagccaacaaggccacactggtgtgtctcata

SEQ ID NO: 103 - DNA, 559 bp, Homo sapiens
  source 1..559: mol_type = other DNA
caatccgccctcactacaaccggatacgccggtctcgaatgtaatacgactcactataggcacgtagtactcgtgactccgacgacaggttacgactcgtcattatcagtcggagcaggagtccgacctcaggtcattaccagtccctccgacacaaaggactggacctacgtctcttagagagactctagggactcccggccaacaataaggacatcctatcctcaaaccctcccaccgggaccacgacgacgtcggttcgacgaggaaccaacggttgttacaacgacaagggtcacgtccactcacaccgccagacagagttcgggaacctgtggctcccaccgactcagtcgggacggaccgcgggagggaggcgacacgggattttactagtcgacccaaggaggtggtttatgttttcacgtagtggtcagcccaaggctgccccctcggtcactctgttcccgccctcctctgaggagcttcaagccaacaaggccacactggtgtgtctcataattcggagacctcccgctactctggcgtcgatgaggga

SEQ ID NO: 104 - RNA, 463 nt, Homo sapiens
  source 1..463: mol_type = other RNA
ggcacgtagtactcgtgactccgacgacaggttacgactcgtcattatcagtcggagcaggagtccgacctcaggtcattaccagtccctccgacacaaaggactggacctacgtctcttagagagactctagggactcccggccaacaataaggacatcctatcctcaaaccctcccaccgggaccacgacgacgtcggttcgacgaggaaccaacggttgttacaacgacaagggtcacgtccactcacaccgccagacagagttcgggaacctgtggctcccaccgactcagtcgggacggaccgcgggagggaggcgacacgggattttactagtcgacccaaggaggtggtttatgttttcacgtagtggtcagcccaaggctgccccctcggtcactctgttcccgccctcctctgaggagcttcaagccaacaaggccacactggtgtgtctcata

SEQ ID NO: 105 - DNA, 562 bp, Homo sapiens
  source 1..562: mol_type = other DNA
caatccgccctcactacaaccggatacgccggtctcgaatgtaatacgactcactataggcacgtagtgcgaacgacgtgtcttcatccaccgactcagaggtccgacccttaggtactatacgttactcttgacgaaccgtgaaaataacttcctctaacacttagaaggaagaaagaaaagtgagtgtagacctgcataccgatagttattacgtcccggaaagggtcccttaacaaccatggtacctttcatcagtttgcgtcacaagagtattcgtgtcaaatattaaccttagggaggaaagacctgatagtttctaactcctgaaacaaagtggacgacgaccgagcacgaaaaacaacgaaagccaacctaaataaaaacatgagtaggggaaaggggttgtcctcgtaatggtcgtgaacacacgtagtatatccagaaccctgaccctgccgtgtaccagctgagagactctaaatccagtgacaagtctgtctgcctattcaccgattttgattattcggagacctcccgctactctggcgtcgatgaggga

SEQ ID NO: 106 - RNA, 466 nt, Homo sapiens
  source 1..466: mol_type = other RNA
ggcacgtagtgcgaacgacgtgtcttcatccaccgactcagaggtccgacccttaggtactatacgttactcttgacgaaccgtgaaaataacttcctctaacacttagaaggaagaaagaaaagtgagtgtagacctgcataccgatagttattacgtcccggaaagggtcccttaacaaccatggtacctttcatcagtttgcgtcacaagagtattcgtgtcaaatattaaccttagggaggaaagacctgatagtttctaactcctgaaacaaagtggacgacgaccgagcacgaaaaacaacgaaagccaacctaaataaaaacatgagtaggggaaaggggttgtcctcgtaatggtcgtgaacacacgtagtatatccagaaccctgaccctgccgtgtaccagctgagagactctaaatccagtgacaagtctgtctgcctattcaccgattttgatt

SEQ ID NO: 107 - DNA, 567 bp, Homo sapiens
  source 1..567: mol_type = other DNA
caatccgccctcactacaaccggatacgccggtctcgaatgtaatacgactcactataggcacgtagtacgaacgacgtgtcttcatccaccgactcagaggtccgacccttaggtactatacgctactcttgacgaaccgtgaaaataacttcctctaacacttagaaggaagaaagaaaagtgagtgtagacctgcataccgatagttattacgtcccggaaagggtcccttaacaaccatggtacctttcatcagtttgcgtcacaagagtattcgtgtcaaatattaactttagggaggaaagacctgatagtttctaactcctgaaacaaagtggacgacgacagacgcgaccagacagagggcgaacccaaagtgtctatcgaggcaagggacaggcttctattcgaaaggagggaccaatatttagtcacgtagtatatccagaaccctgaccctgccgtgtaccagctgagagactctaaatccagtgacaagtctgtctgcctattcaccgattttgattattcggagacctcccgctactctggcgtcgatgaggga

SEQ ID NO: 108 - RNA, 471 nt, Homo sapiens
  source 1..471: mol_type = other RNA
ggcacgtagtacgaacgacgtgtcttcatccaccgactcagaggtccgacccttaggtactatacgctactcttgacgaaccgtgaaaataacttcctctaacacttagaaggaagaaagaaaagtgagtgtagacctgcataccgatagttattacgtcccggaaagggtcccttaacaaccatggtacctttcatcagtttgcgtcacaagagtattcgtgtcaaatattaactttagggaggaaagacctgatagtttctaactcctgaaacaaagtggacgacgacagacgcgaccagacagagggcgaacccaaagtgtctatcgaggcaagggacaggcttctattcgaaaggagggaccaatatttagtcacgtagtatatccagaaccctgaccctgccgtgtaccagctgagagactctaaatccagtgacaagtctgtctgcctattcaccgattttgatt

SEQ ID NO: 109 - DNA, 540 bp, Homo sapiens
  source 1..540: mol_type = other DNA
caatccgccctcactacaaccggatacgccggtctcgaatgtaatacgactcactataggcacgtagttgtcgcgtcttcattctccgtctcagaaagtagacctcgaggacatcttcctccattggtattgatagtctcgctgattccttacttctttttgctggacagaggaggtttggtaggtctggtaacattctttctttacacccacgaggcggtagtacaacgaccatggtcctgtcgggtattttgggtctacagaccatgcacgtcaaatagacctgttaccgaggaaggtgtcgacagtgaagtctcccgacgagttccgaaacagggcaccaaaccattctaggtggaaattgtaccagggggaacgtttctcgtcgaagaccggtagacttttggcacgtagtatatccagaaccctgaccctgccgtgtaccagctgagagactctaaatccagtgacaagtctgtctgcctattcaccgattttgattattcggagacctcccgctactctggcgtcgatgaggga

SEQ ID NO: 110 - RNA, 444 nt, Homo sapiens
  source 1..444: mol_type = other RNA
ggcacgtagttgtcgcgtcttcattctccgtctcagaaagtagacctcgaggacatcttcctccattggtattgatagtctcgctgattccttacttctttttgctggacagaggaggtttggtaggtctggtaacattctttctttacacccacgaggcggtagtacaacgaccatggtcctgtcgggtattttgggtctacagaccatgcacgtcaaatagacctgttaccgaggaaggtgtcgacagtgaagtctcccgacgagttccgaaacagggcaccaaaccattctaggtggaaattgtaccagggggaacgtttctcgtcgaagaccggtagacttttggcacgtagtatatccagaaccctgaccctgccgtgtaccagctgagagactctaaatccagtgacaagtctgtctgcctattcaccgattttgatt

SEQ ID NO: 111 - DNA, 580 bp, Homo sapiens
  source 1..580: mol_type = other DNA
caatccgccctcactacaaccggatacgccggtctcgaatgtaatacgactcactataggcacgtagtggtggcgatgacatctctatgtgccgactcagggagccgagacgccctacctagaagtcacaccttctgcaaggcaatccagaaagtcgacacttagcaaggaacccgtagggacttagttgacgttacaaaaccatgaccttcctttagtcgttgaagtaaagaacggagtctaagacagccatggtcacttgtttgtacactggtcttctacctagcgtggagtcttagtggaggacagggtacaaccactggaacacagacccccagaccgactgaggtccgagaaaaagcacgaagacacgggggacagggggcgcagacacagagggtcttggtcctcaggaggcgggtctgcgggtcgtcgcggagagtccacgtagtgaggacctgaacaaggtgttcccacccgaggtcgctgtgtttgagccatcagaagcagagatctcccacacccaaaaggccacactggtgattcggagacctcccgctactctggcgtcgatgaggga

SEQ ID NO: 112 - RNA, 484 nt, Homo sapiens
  source 1..484: mol_type = other RNA
ggcacgtagtggtggcgatgacatctctatgtgccgactcagggagccgagacgccctacctagaagtcacaccttctgcaaggcaatccagaaagtcgacacttagcaaggaacccgtagggacttagttgacgttacaaaaccatgaccttcctttagtcgttgaagtaaagaacggagtctaagacagccatggtcacttgtttgtacactggtcttctacctagcgtggagtcttagtggaggacagggtacaaccactggaacacagacccccagaccgactgaggtccgagaaaaagcacgaagacacgggggacagggggcgcagacacagagggtcttggtcctcaggaggcgggtctgcgggtcgtcgcggagagtccacgtagtgaggacctgaacaaggtgttcccacccgaggtcgctgtgtttgagccatcagaagcagagatctcccacacccaaaaggccacactggtg

SEQ ID NO: 113 - DNA, 554 bp, Homo sapiens
  source 1..554: mol_type = other DNA
caatccgccctcactacaaccggatacgccggtctcgaatgtaatacgactcactataggcacgtagtcgacgaccgtgtctctatgtaccggctcaggggttcgagacgaccgacctagaactctcacctcagatgaggaaactcggagagacgtcttttagctaggaatccgttgacacttagcagatgacgaaggagtaagagtatagcttagtcttcgaggcccgggacagggttcaagacgtccatggtcatttcccataacaccggtctttaacctaacgtggtttttcggtgtccgacaaagaagagatattagaatatagaccctctgggaaaggacggacgggggacagggaccgagccgatcctacctctcagctcagggtagtggttttacgaccccgactaacgatcacgtagtgaggacctgaacaaggtgttcccacccgaggtcgctgtgtttgagccatcagaagcagagatctcccacacccaaaaggccacactggtgattcggagacctcccgctactctggcgtcgatgaggga

SEQ ID NO: 114 - RNA, 458 nt, Homo sapiens
  source 1..458: mol_type = other RNA
ggcacgtagtcgacgaccgtgtctctatgtaccggctcaggggttcgagacgaccgacctagaactctcacctcagatgaggaaactcggagagacgtcttttagctaggaatccgttgacacttagcagatgacgaaggagtaagagtatagcttagtcttcgaggcccgggacagggttcaagacgtccatggtcatttcccataacaccggtctttaacctaacgtggtttttcggtgtccgacaaagaagagatattagaatatagaccctctgggaaaggacggacgggggacagggaccgagccgatcctacctctcagctcagggtagtggttttacgaccccgactaacgatcacgtagtgaggacctgaacaaggtgttcccacccgaggtcgctgtgtttgagccatcagaagcagagatctcccacacccaaaaggccacactggtg

SEQ ID NO: 115 - DNA, 579 bp, Homo sapiens
  source 1..579: mol_type = other DNA
caatccgccctcactacaaccggatacgccggtctcgaatgtaatacgactcactataggcacgtagtagaaccgacgaccgcgtctctatgtcccggctcagaagaccgacgtcccacacatccacttccttattcactctcgacaaccccgtaagtccactcttcgctgaaccgtgtgacaacaaaagttgggcaagaagttctgacatctgtttgtactcgaggtcaccgaagaatcgtgaaacgaacatggttatgtatcgcaatactgggtctacaacaagtgtaaagtttctgaagaataaacagtaagggtactggtccacagaaccacagacgcattgagggcaaagccagccggccgcgggagggcgatcagggcagggaccaggatcctacctctcagctcagggtagtggttttacgaccccgactaacgatcacgtagtgaggacctgaacaaggtgttcccacccgaggtcgctgtgtttgagccatcagaagcagagatctcccacacccaaaaggccacactggtgattcggagacctcccgctactctggcgtcgatgaggga

SEQ ID NO: 116 - RNA, 483 nt, Homo sapiens
  source 1..483: mol_type = other RNA
ggcacgtagtagaaccgacgaccgcgtctctatgtcccggctcagaagaccgacgtcccacacatccacttccttattcactctcgacaaccccgtaagtccactcttcgctgaaccgtgtgacaacaaaagttgggcaagaagttctgacatctgtttgtactcgaggtcaccgaagaatcgtgaaacgaacatggttatgtatcgcaatactgggtctacaacaagtgtaaagtttctgaagaataaacagtaagggtactggtccacagaaccacagacgcattgagggcaaagccagccggccgcgggagggcgatcagggcagggaccaggatcctacctctcagctcagggtagtggttttacgaccccgactaacgatcacgtagtgaggacctgaacaaggtgttcccacccgaggtcgctgtgtttgagccatcagaagcagagatctcccacacccaaaaggccacactggtg

SEQ ID NO: 117 - DNA, 568 bp, Homo sapiens
  source 1..568: mol_type = other DNA
caatccgccctcactacaaccggatacgccggtctcgaatgtaatacgactcactataggcacgtagttcaaggggttctcgtgttttcatgaaacgacttagaagatcgacattccgactttaccaattccgctgcctaaagcgacgaaagaacttcaactgtcttatcgctggtgaaaaacgtaagacaagtagtcttgggaccgcttattccttttagtagagaaacgacccttcaacgaacatggttttttatattatactggtggttgaacaaagtatgtccgtcaagtcccactgacgaaaggagtgaccgtacctatgactactgacccgaactcattggaagacccgcgacaagtgataaagaccacccccgaggtgcttctcaaactacggtcaaggctttttgtagacagcccacagggtcctccacgtagtgaagtcagcctcataccaaaccatccgtttttgtcatgaaaaatggaacaaatgtcgcttgtctggtgaaggaattctaccccaaggaattcggagacctcccgctactctggcgtcgatgaggga

SEQ ID NO: 118 - RNA, 472 nt, Homo sapiens
  source 1..472: mol_type = other RNA
ggcacgtagttcaaggggttctcgtgttttcatgaaacgacttagaagatcgacattccgactttaccaattccgctgcctaaagcgacgaaagaacttcaactgtcttatcgctggtgaaaaacgtaagacaagtagtcttgggaccgcttattccttttagtagagaaacgacccttcaacgaacatggttttttatattatactggtggttgaacaaagtatgtccgtcaagtcccactgacgaaaggagtgaccgtacctatgactactgacccgaactcattggaagacccgcgacaagtgataaagaccacccccgaggtgcttctcaaactacggtcaaggctttttgtagacagcccacagggtcctccacgtagtgaagtcagcctcataccaaaccatccgtttttgtcatgaaaaatggaacaaatgtcgcttgtctggtgaaggaattctaccccaagga

SEQ ID NO: 119 - DNA, 577 bp, Homo sapiens
  source 1..577: mol_type = other DNA
caatccgccctcactacaaccggatacgccggtctcgaatgtaatacgactcactataggcacgtagttcaaggggttctcgtgttttcatgaaacgacttagaagatcgacattccgactttaccaattccgctgcctaaagcgacgaaagaacttcaactgtcttatcgctggtgaaaaacgtaagacaagtagtcttgggaccgcttattccttttagtagagaaacgacccttcaacgaacatggttttttatattatactggtggttgaacaaagtatgtccgtcaagtcccactgacgaaaggagtgaccgtacctatgactactgacccgaactcattggaagacccgaaggcagaccgccaaggtgataaagaacaacgccgaccaaggtgctactcaacacaagggaaaggtttcttctcaacacgacagtttccacgtagtgaagtcagcctcataccaaaccatccgtttttgtcatgaaaaatggaacaaatgtcgcttgtctggtgaaggaattctaccccaaggaattcggagacctcccgctactctggcgtcgatgaggga

SEQ ID NO: 120 - RNA, 481 nt, Homo sapiens
  source 1..481: mol_type = other RNA
ggcacgtagttcaaggggttctcgtgttttcatgaaacgacttagaagatcgacattccgactttaccaattccgctgcctaaagcgacgaaagaacttcaactgtcttatcgctggtgaaaaacgtaagacaagtagtcttgggaccgcttattccttttagtagagaaacgacccttcaacgaacatggttttttatattatactggtggttgaacaaagtatgtccgtcaagtcccactgacgaaaggagtgaccgtacctatgactactgacccgaactcattggaagacccgaaggcagaccgccaaggtgataaagaacaacgccgaccaaggtgctactcaacacaagggaaaggtttcttctcaacacgacagtttccacgtagtgaagtcagcctcataccaaaccatccgtttttgtcatgaaaaatggaacaaatgtcgcttgtctggtgaaggaattctaccccaagga

SEQ ID NO: 121 - DNA, 557 bp, Homo sapiens
  source 1..557: mol_type = other DNA
caatccgccctcactacaaccggatacgccggtctcgaatgtaatacgactcactataggcacgtagttcaaggggttctcgtgttttcatgaaacgacttagaagatcgacattccgactttaccaattccgctgcctaaagcgacgaaagaacttcaactgtcttatcgctggtgaaaaacgtaagacaagtagtcttgggaccgcttattccttttagtagagaaacgacccttcaacgaacatggttttttatattatactggtggttgaacaaagtatgtccgtcaagtcccactgacgaaaggagtgaccgtacctatgactactgacccgaactcattggaagacccgagccgatgataaagacagaacaacaacatggaggtctatccaaggaaacggtttctagtccccagacccacgtagtgaagtcagcctcataccaaaccatccgtttttgtcatgaaaaatggaacaaatgtcgcttgtctggtgaaggaattctaccccaaggaattcggagacctcccgctactctggcgtcgatgaggga

SEQ ID NO: 122 - RNA, 461 nt, Homo sapiens
  source 1..461: mol_type = other RNA
ggcacgtagttcaaggggttctcgtgttttcatgaaacgacttagaagatcgacattccgactttaccaattccgctgcctaaagcgacgaaagaacttcaactgtcttatcgctggtgaaaaacgtaagacaagtagtcttgggaccgcttattccttttagtagagaaacgacccttcaacgaacatggttttttatattatactggtggttgaacaaagtatgtccgtcaagtcccactgacgaaaggagtgaccgtacctatgactactgacccgaactcattggaagacccgagccgatgataaagacagaacaacaacatggaggtctatccaaggaaacggtttctagtccccagacccacgtagtgaagtcagcctcataccaaaccatccgtttttgtcatgaaaaatggaacaaatgtcgcttgtctggtgaaggaattctaccccaagga

SEQ ID NO: 123 - DNA, 567 bp, Homo sapiens
  source 1..567: mol_type = other DNA
caatccgccctcactacaaccggatacgccggtctcgaatgtaatacgactcactataggcacgtagtgtcccgggtccaacacgtcatcaagtacgggagctacggacagaggttgaagtcatgacggttctatctcgacgacggtaggaaccgatactaaaatagacgaaagtcctaccttagggtgacgtgtaggttgaacctgtaccggtcgaccacctcacggagtccctggaccgggaagacgaccatggtcacccacatcgactaggaccacgcctggtgcgtgacgtcctactggttcgaagggatcacggggtcctgtacccggtcgacgaggccggactactccagaaaggaacaccggggaaggcagaaggacactgttgttcacaacaaggtgacggtttctcaaagaatattattaagcacgtagtgataaacaacttgatgcagatgtttcccccaagcccactatttttcttccttcgattgctgaaacaaaactccagaaggctggaacatacattcggagacctcccgctactctggcgtcgatgaggga

SEQ ID NO: 124 - RNA, 471 nt, Homo sapiens
  source 1..471: mol_type = other RNA
ggcacgtagtgtcccgggtccaacacgtcatcaagtacgggagctacggacagaggttgaagtcatgacggttctatctcgacgacggtaggaaccgatactaaaatagacgaaagtcctaccttagggtgacgtgtaggttgaacctgtaccggtcgaccacctcacggagtccctggaccgggaagacgaccatggtcacccacatcgactaggaccacgcctggtgcgtgacgtcctactggttcgaagggatcacggggtcctgtacccggtcgacgaggccggactactccagaaaggaacaccggggaaggcagaaggacactgttgttcacaacaaggtgacggtttctcaaagaatattattaagcacgtagtgataaacaacttgatgcagatgtttcccccaagcccactatttttcttccttcgattgctgaaacaaaactccagaaggctggaacatac

SEQ ID NO: 125 - DNA, 592 bp, Homo sapiens
  source 1..592: mol_type = other DNA
caatccgccctcactacaaccggatacgccggtctcgaatgtaatacgactcactataggcacgtagtcacggattaggtcgtccgtgtcaccatgtggtggagtagaagaaagagattcttgaaataaaagtttcaccttcaccttcacactcgtaaaaatgaatagagttcaaagaatcagaagggtggactcgttagaactcgtctctaacagttcttctgtacattatttataagattcggaaacaaaccaaagacggtcatggtcacatactaaaatgacgatttcggaacctacctacggaaggttctatacacccgtgagaataaacgaccagacctttatctataaagtccaacaaggttgacgggttcgaacgccacgggaaggagcccacgccgaagacactgttgttcacaacaaggtgacggtttctcaaagaatattattaagcacgtagtgataaacaacttgatgcagatgtttcccccaagcccactatttttcttccttcgattgctgaaacaaaactccagaaggctggaacatacattcggagacctcccgctactctggcgtcgatgaggga

SEQ ID NO: 126 - RNA, 496 nt, Homo sapiens
  source 1..496: mol_type = other RNA
ggcacgtagtcacggattaggtcgtccgtgtcaccatgtggtggagtagaagaaagagattcttgaaataaaagtttcaccttcaccttcacactcgtaaaaatgaatagagttcaaagaatcagaagggtggactcgttagaactcgtctctaacagttcttctgtacattatttataagattcggaaacaaaccaaagacggtcatggtcacatactaaaatgacgatttcggaacctacctacggaaggttctatacacccgtgagaataaacgaccagacctttatctataaagtccaacaaggttgacgggttcgaacgccacgggaaggagcccacgccgaagacactgttgttcacaacaaggtgacggtttctcaaagaatattattaagcacgtagtgataaacaacttgatgcagatgtttcccccaagcccactatttttcttccttcgattgctgaaacaaaactccagaaggctggaacatac

SEQ ID NO: 127 - DNA, 604 bp, Homo sapiens
  source 1..604: mol_type = other DNA
caatccgccctcactacaaccggatacgccggtctcgaatgtaatacgactcactataggcacgtagtcacggattaggtcgtccgtgtcaccatgtggtggagtagaagaaagagattcttgaaataaaagtttcaccttcaccttcacactcgtaaaaatgaatagagttcaaagaatcagaagggtggactcgttagaactcgtctctaacagttcttctgtacattatttataagattcggaaacaaaccaaagacggtcatggtcacatactaaaatgacgatttcggaacctacctacggaaggttctatacacccgtgagaataaacgaccagacctttatctataaagtccaacaaggttgacgggttcgcacccgaaaaagcccacgccagcgcagaacgtccgcttcaatgatactcggatcagggaaaacgtttgcagaactaggttagtgatgatacacgtagtgataaacaacttgatgcagatgtttcccccaagcccactatttttcttccttcgattgctgaaacaaaactccagaaggctggaacatacattcggagacctcccgctactctggcgtcgatgaggga

SEQ ID NO: 128 - RNA, 508 nt, Homo sapiens
  source 1..508: mol_type = other RNA
ggcacgtagtcacggattaggtcgtccgtgtcaccatgtggtggagtagaagaaagagattcttgaaataaaagtttcaccttcaccttcacactcgtaaaaatgaatagagttcaaagaatcagaagggtggactcgttagaactcgtctctaacagttcttctgtacattatttataagattcggaaacaaaccaaagacggtcatggtcacatactaaaatgacgatttcggaacctacctacggaaggttctatacacccgtgagaataaacgaccagacctttatctataaagtccaacaaggttgacgggttcgcacccgaaaaagcccacgccagcgcagaacgtccgcttcaatgatactcggatcagggaaaacgtttgcagaactaggttagtgatgatacacgtagtgataaacaacttgatgcagatgtttcccccaagcccactatttttcttccttcgattgctgaaacaaaactccagaaggctggaacatac

(19) United States
(12) Patent Application Publication
(10) Pub. No.: US 2026/0347925 A1
(43) Pub. Date: Nov. 5, 2026

(54) Methods for Characterizing an Immune Response Repertoire

(71) Applicant: NEW ENGLAND BIOLABS, INC., Ipswich, MA (US)

(72) Inventors: Chen Song, Boxford, MA (US); Ariel Erijman, Ipswich, MA (US); Sean Lund, Boston, MA (US); Bradley W. Langhorst, Beverly, MA (US); Pingfang Liu, Hamilton, MA (US)

(73) Assignee: NEW ENGLAND BIOLABS, INC., Ipswich, MA (US)

(21) Appl. No.: 19/113,590

(22) PCT Filed: Sep. 20, 2023
(86) PCT No.: PCT/US2023/074661

§ 371 Date: Mar. 20, 2025

Related U.S. Application Data
(60) Provisional application No. 63/376,354, filed on Sep. 20, 2022.
(60) Provisional application No. 63/377,068, filed on Sep. 26, 2022.

(51) Int. Cl. C12Q 1/6869; C12N 15/10; C12Q 1/6806; C12Q 1/6855

(52) U.S. Cl. CPC C12Q 1/6869; C12N 15/1065; C12Q 1/6806; C12Q 1/6855

(57) ABSTRACT

This disclosure provides synthetic nucleic acid oligonucleotides and related methods for analysis of immune repertoires. A disclosed synthetic oligonucleotide contains (a) one or more tags with a sequence that is not contained in a eukaryotic immune repertoire, wherein at least one tag is positioned between a region corresponding to a diversity and joining region ((D)J) and a constant region (C) of an immunoglobulin (IG) or T cell receptor, thereby distinguishing the synthetic oligonucleotide from naturally occurring sequences in the eukaryotic immune repertoire; and wherein the V(D)J region has a predetermined V(D)J clonotype, the synthetic oligonucleotide optionally further comprising a tag proximate to a region corresponding to a eukaryotic variable region (V); and optionally wherein C further comprises one or more priming sites for nucleic acid amplification.

### BACKGROUND

Immune sequencing technologies seek to determine an immune repertoire of an individual as a means to track the dynamics of disease and treatment by capturing the diversity of a B-cell and/or T-cell response through generating and then sequencing DNA or RNA libraries. While DNA libraries have been frequently used, RNA copy number is important in estimating clonal expansion and identifying rare clones. Lack of standard references causes difficulty in assessing reproducibility, sensitivity and accuracy of immune sequencing technologies applied to RNA.

### SUMMARY

Provided herein is a synthetic nucleic acid oligonucleotide, which contains (a) one or more tags with a sequence that is not contained in a eukaryotic immune repertoire, wherein at least one tag is positioned between a region corresponding to a diversity and joining region ((D)J) and a constant region (C) of an immunoglobulin (IG) or T cell receptor, thereby distinguishing the synthetic oligonucleotide from naturally occurring sequences in the eukaryotic immune repertoire; and wherein the V(D)J region has a predetermined V(D)J clonotype, the synthetic oligonucleotide optionally further comprising a tag proximate to a region corresponding to a eukaryotic variable region (V); and optionally wherein C further comprises one or more priming sites for nucleic acid amplification.

The synthetic oligonucleotide may be a DNA oligonucleotide. In an embodiment, the DNA oligonucleotide is a single stranded DNA; in another embodiment the DNA oligonucleotide is a double stranded DNA. The DNA oligonucleotide may optionally include a promoter sequence for a DNA dependent RNA polymerase. In this embodiment, one of the tags may optionally be positioned between the promoter and the variable region. In an embodiment, the DNA oligonucleotide may further include a poly-dT tail.

In an embodiment, the synthetic oligonucleotide may have a length in the range of 200-1000 nucleotides, for example in the range of 450-700 nucleotides. In an embodiment, the tag may have a length in the range of 6-25 nucleotides, e.g., 8-12 nucleotides, such as 8 nucleotides.

In an embodiment, the synthetic oligonucleotide may be an RNA oligonucleotide. The RNA oligonucleotide may optionally include a poly-A tail. The RNA oligonucleotide may optionally be capped, or the transcribed RNA may be capped if the synthetic oligonucleotide is DNA.

In an embodiment, the synthetic oligonucleotide may further include sequencing adaptors.

In an embodiment, the eukaryotic immune repertoire is a mammalian immune repertoire, and may be a human immune repertoire.

In an embodiment, the sequence of the V(D)J region comprises naturally occurring sequence. In an embodiment, the sequence of the V(D)J region comprises non-naturally occurring, designed sequence.

In an embodiment, the synthetic oligonucleotide is contained within a mixture of synthetic oligonucleotides, wherein each oligonucleotide in the mixture contains a different V(D)J clonotype. The mixture of clonotypes may include each of at least 2, e.g., 5 different clonotypes.

In an embodiment, a plurality of mixtures of clonotypes are combined to form an internal control for a eukaryotic derived sample.

In an embodiment, the plurality of mixtures of clonotypes comprises at least 2 mixtures, e.g., 10 mixtures, for forming a control pool.

Also provided is a method for RNA analysis of an immune response. The method involves:
(a) obtaining a synthetic oligonucleotide as described herein;
(b) optionally performing in vitro transcription of the synthetic oligonucleotide if the synthetic oligonucleotide is DNA and omitting this step if the synthetic oligonucleotide is RNA;
(c) optionally performing a capping reaction if the synthetic oligonucleotide is RNA, or optionally performing a capping reaction on the transcribed RNA if the synthetic oligonucleotide is DNA;
(d) forming a control pool of RNA oligonucleotides from the DNA oligonucleotides in (b) or from the RNA oligonucleotides in (a) or (c) wherein the control pool of RNA oligonucleotides comprises a plurality of mixtures of clonotypes wherein each mixture comprises a plurality of clonotypes at different predetermined concentrations;
(e) adding a predetermined concentration of the control pool of RNA from (d) to a sample obtained from a eukaryote comprising RNAs produced by an immune response; and
(f) preparing a library of (e) for sequencing.

In an embodiment, the method involves adding a polynucleotide tail to the synthetic oligonucleotide by means of a primer and amplifying the primed oligonucleotide.

In an embodiment, the method further involves sequencing the RNA from (f) to analyze the RNA associated with the immune response.

In an embodiment, the method further involves identifying the control pool of the synthetic oligonucleotides.

In an embodiment, the method further involves using the identified control pool for a purpose selected from quantifying the eukaryotic RNA associated with immune response; normalizing the eukaryotic RNA associated with immune response; determining the sensitivity of an immune repertoire technology; and determining the sensitivity of an immune repertoire technology.

### DESCRIPTION OF FIGURES

FIG. 1 shows the design of a suitable DNA sequence for a transcribed DNA oligonucleotide control.

FIG. 2 shows the workflow starting with design of a DNA oligonucleotide for making an RNA control and its use in a sequencing library.

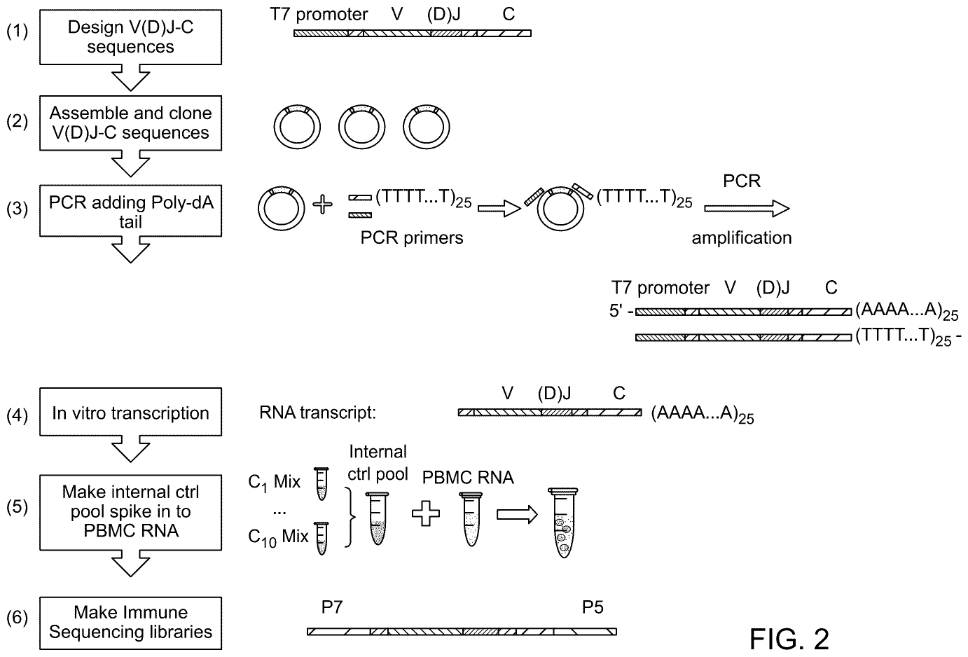

FIG. 3 shows clonotypes and their concentration in two examples of mixes of constant (C) region priming sites: Immunoglobulin heavy chain (IGH) (all the heavy chain isotypes), Immunoglobulin light chain (lambda chain or kappa chain, IGL/K) and T cell receptor (alpha, beta, delta and gamma chains, TRA/B/D/G). The control pool contains a plurality of mixtures of different C regions where each mixture is a plurality of different C regions combined at a defined molar ratio. For example, each C region priming site is shown here to contain 5 different V(D)J sequences although more or fewer V(D)J sequences may be contained in C. Each Cₙ mix in this example is a combination of different V(D)J clonal types from the same C region in a broad dynamic range from 5-0.0005 nM. Equal volumes of C₁ Mix, C₂ Mix, . . . . C₁₀ Mixes are then combined to make an internal control pool (while n=10 is used here as an example. n can by any number greater than 1). The control mix was then spiked into a peripheral blood mononuclear cells (PBMC) RNA sample to serve as an internal control pool and Immune sequencing libraries were then made and sequenced following the manufacturer's instructions (see NEBNext Immune Sequencing kit, New England Biolabs, Ipswich, MA).

FIG. 4A-4C shows clonotype correlations between two replicates of libraries made using the NEBNext® Immune Sequencing Kit (New England Biolabs, Ipswich, MA). FIG. 4A is a plot of 29 IG clonotype controls, R²=0.9604; FIG. 4B is a plot of 20 TCR clonotype controls, R²=0.9964; FIG. 4C is a plot of PBMC RNA, R²=0.7408. Correlation analysis between two replicates on the clonotype counts from internal controls, show superior clonotype correlation across high and low abundance clonotypes with large dynamic range. In comparison, libraries formed from PBMC RNA in the absence of controls show much poorer correlation with each other. Reads from internal controls were extracted from total reads using the two 8 nucleotide tags shown in FIG. 1 for DNA oligonucleotides or a similar single tag for RNA oligonucleotides (680,000 sampled from each library), processed by The REpertoire Sequencing TOolkit (pRESTO), and mapped to reference sequences for internal controls, resulting in counts for each clonotype.

### DETAILED DESCRIPTION

The following brief explanation of immunoglobulins is intended to provide background for the present disclosure. In the developing B cell, the first recombination event to occur is between one diversity (D) gene segment and one joining (J) gene segment of the heavy chain locus. Any DNA between these two gene segments is deleted. This D-J recombination is followed by the joining of one variable (V) gene segment, from a region upstream of the newly formed DJ complex, forming a rearranged VDJ gene segment. All other gene segments between V and D segments are now deleted from the cell's genome. Primary transcript (unspliced RNA) is generated containing the VDJ region of the heavy chain and both the constant mu and delta chains (Cμ and Cδ). (i.e. the primary transcript contains the segments: V-D-J-Cμ-Cδ). The primary RNA is processed to add a polyadenylated (poly-A) tail after the Cμ chain and to remove sequence between the VDJ segment and the constant gene segment. Translation of this mRNA leads to the production of the IgM heavy chain protein.

In the formation of the light chain, the kappa (κ) and lambda (λ) chains of the immunoglobulin light chain loci rearrange in a very similar way, except that the light chains lack a D segment. In other words, the first step of recombination for the light chains involves the joining of the V and J chains to give a VJ complex before the addition of the constant chain gene during primary transcription.

Translation of the spliced mRNA for either the kappa or lambda chains results in formation of the Ig κ or Ig λ light chain protein.

Assembly of the Ig μ heavy chain and one of the light chains results in the formation of membrane bound form of the immunoglobulin IgM that is expressed on the surface of the immature B cell. A clonotype is a unique nucleotide sequence that arises during the above-mentioned gene rearrangement. For example, the circulating repertoire of each human individual contained between 9 and 17 million B cell clonotypes (see for example, Soto et al. (2019) Nature, 566, 398-402). A combination of nucleotide sequences for the surface expressed receptor pair would define the T cell clonotype (see for example, Straten et al. (2004) Journal of Translational Medicine, 2, 1-10; Yassai et al. Immunogenetics (2009) 61, 493-502).

### Synthetic Oligonucleotides

Provided herein is a synthetic nucleic acid oligonucleotide, which contains (a) one or more tags with a sequence that is not contained in a eukaryotic immune repertoire, wherein at least one tag is positioned between a region corresponding to a diversity and joining region ((D)J) and a constant region (C) of an immunoglobulin (IG) or T cell receptor, thereby distinguishing the synthetic oligonucleotide from naturally occurring sequences in the eukaryotic immune repertoire; and wherein the V(D)J region has a predetermined V(D)J clonotype, the synthetic oligonucleotide optionally further comprising a tag proximate to a region corresponding to a eukaryotic variable region (V); and optionally wherein C further comprises one or more priming sites for nucleic acid amplification.

The length of a synthetic oligonucleotide may be in the range of 200-1000 nucleotides, for example in the range of 450-700 nucleotides. The oligonucleotide may be double stranded DNA, single stranded DNA, a combination thereof, or RNA. The oligonucleotide may be linear or may be circular. The circular oligonucleotides may be incorporated in a vector, e.g., for purposes of cloning and production of reagent quantities by fermentation. Linear and circular oligonucleotides may be amplified using a variety of methods, e.g., by PCR (for DNA) or rolling circle amplification (for DNA or RNA) (see for example, US20230141630A1 for rolling circle amplification of RNA).

Where the oligonucleotide is DNA, a poly-dT tail (for example 20 Ts) may added prior to transcription with an RNA polymerase. A poly-dT tail may be added, for example, by ligation or amplification using a primer having a poly-dT tail; or by in vitro transcription. Where the oligonucleotide is RNA, a poly-A tail (for example, 20 As) may be added by ligation, or by a Poly(A) polymerase to the end of the oligonucleotide.

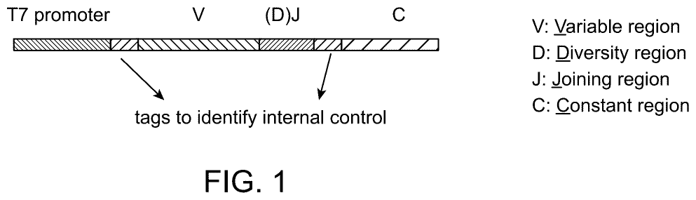

A DNA oligonucleotide may contain an RNA polymerase promoter such as a phage promoter (e.g., a phage T7 promoter) suitable for initiating in vitro transcription. At least one tag of at least 6 nucleotides in length, preferably 8 nucleotides or more, such as 8-12 nucleotides, and for example 8 nucleotides, may be included in a DNA oligonucleotide sequence in order to differentiate the controls from real sample clonotypes. Preferably a tag is positioned between the T7 promoter region and the V region and a second tag is positioned between the (D)J region and the C region as shown in FIG. 1. If the oligonucleotide is an RNA oligonucleotide, then a single tag may be positioned between the (D)J region and the C region. Alternatively, a second tag may be positioned before the V region of a RNA oligonucleotide in addition to a first tag positioned between the (D)J region and the C region. The RNA oligonucleotide may be designed to omit the phage promoter region for a DNA dependent RNA polymerase used in transcription of the DNA although a suitable promoter region may be included for an RNA dependent RNA polymerase for copying the RNA oligonucleotide.

An oligonucleotide may include an element to improve stability, if desired. For example, when the oligonucleotide is RNA or transcribed RNA if the synthetic oligonucleotide is DNA, it may be capped. Any of a variety of capping procedures and enzymes may be used, e.g., Faustovirus Capping Enzyme (M2081; New England Biolabs, Ipswich, MA).

The sequence for the V(D)J combination incorporated into the oligonucleotide design is selected as representative of clonotypes from healthy donors (see Table 2, Natural V(D)J combination). The constant region positioned 3′ of the V and (D)J regions also contains NEBNext Immune Sequencing priming sites. Each constant region contains at least 2, preferably 5 different V(D)J clonotypes. Alternatively, the V(D)J region may contain non-naturally occurring sequences, such as in-silico designed sequence that mimics the eukaryotic immune repertoire in sequence diversity and structural context (See Table 2 for exemplary sequences). Such sequences can be designed to have predictable performance in downstream applications. The in-silico designed sequences may be selected from any combination of reverse complemented natural V, D and J segment sequences, random sequences, or other V,D,J+CDR sequences, which are further selected by comparing sequence context to provide higher V(D)J diversity.

The synthetic oligonucleotides may be combined into a plurality of mixtures of clonotypes. Each mixture may include a plurality of clonotypes at different predetermined concentrations. A mixture of clonotypes may be selected from the synthetic oligonucleotide sequences of Table 2. The predetermined concentrations are selected to represent a broad dynamic range (e.g,, from 5-0.0005 nM) as determined by a quantitative method, e.g., by Nanodrop or RT-qPCR.

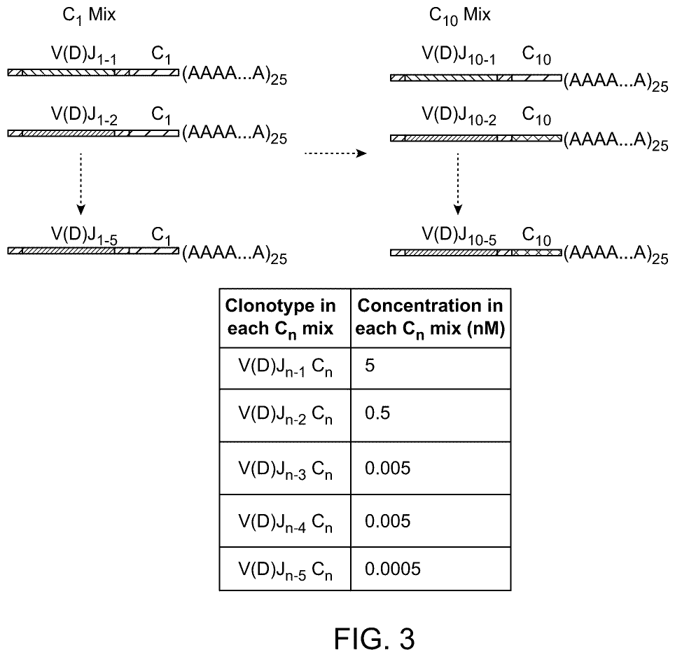

As one example, the sequences for DNA oligonucleotides were selected as follows: (1) Synthetic DNA templates were ordered and assembled into plasmid for cloning (2) PCR amplification of plasmid added Poly-dA tail (3) In vitro transcription (IVT) resulted in control RNA templates (4) The RNA product of IVT was quantified and validated with NEBNext Immune Sequencing workflow (New England Biolabs, Ipswich, MA). Mixes and control pools were created as shown in FIG. 3. The control pool was spiked into PBMC RNA (5) and the NEBNext Immune Sequencing libraries (E6320, E6330) were made using control pool spiked PBMC RNA (6).

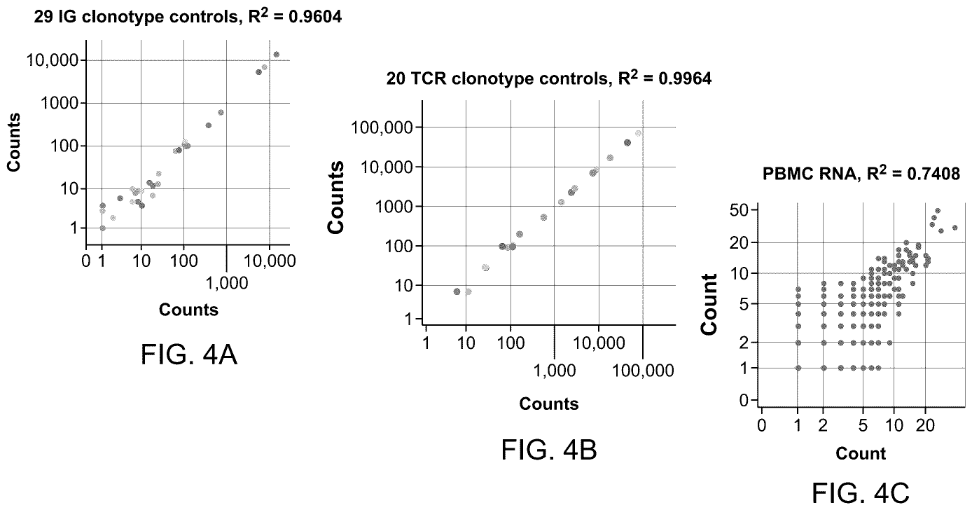

Absence of such controls makes quantitative data analysis more challenging, e.g. it is more difficult to differentiate between technical and biological variation without controls, as exemplified in FIG. 4. Moreover, with controls, reproducibility between non-replicate libraries, can be assessed with high confidence (e.g. longitudinal samples from the same individual).

Applications for Cₙ mixes and control pools

Features of mixtures of synthetic oligonucleotides described herein (e.g., NEBNext Immune Sequencing RNA controls) include:
(i) A pool of many sequences to represent V(D)J clonotype diversity and different constant region isotypes
(ii) Screening candidate sequences throughout the whole workflow (from library preparation to data analysis) to ensure reproducibility of the sequencing data
(iii) One or more tag sequences in each of the oligonucleotides used in the controls to differentiate the controls from real sample clonotypes
(iv) Quantitatively mixed sequences to span high dynamic range of at least 2 orders of magnitude such as 5 orders of magnitude for sensitivity and accuracy assessment

Applications include the following: standard reference for RT-qPCR, loop-mediated isothermal amplification (LAMP), strand displacement amplification (SDA), and helicase-dependent amplification (HAD) based methods; synthetic RNA controls for RNA modification detection; and spike-in controls for Immune sequencing library prep workflows designed for other sequencing platforms, such as MGI, PacBio, Oxford Nanopore, and other new platforms.

Examples of sequences of representative immune repertoire controls are provided in Table 2. The following Table 1 provides concordance in naming between the presently filed application (“Updated name”) and corresponding provisional application (“Original name”).

TABLE 1
Sequence Concordance

| Original name | Updated name |
|---|---|
| HTman-IGHA_0 | Human-IGHA_0 |
| HTman-IGHD_0 | Human-IGHD_0 |
| HTman-IGHE_2 | Human-IGHE_2 |
| HTman-IGHG_0 | Human-IGHG_0 |
| HTman-IGK_0 | Human-IGK_0 |
| HTman-IGL_0 | Human-IGL_0 |
| HTman-TRA_0 | Human-TRA_0 |
| HTman-TRB_0 | Human-TRB_0 |
| HTman-TRD_0 | Human-TRD_0 |
| HTman-TRG_0 | Human-TRG_0 |

Unless otherwise defined, all technical and scientific terms used herein have the same meaning as commonly understood by one of ordinary skill in the art to which this disclosure belongs. Still, certain terms are defined herein with respect to embodiments of the disclosure and for the sake of clarity and ease of reference.

Sources of commonly understood terms and symbols may include: standard treatises and texts such as Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); Singleton, et al., Dictionary of Microbiology and Molecular biology, 2d ed., John Wiley and Sons, New York (1994), and Hale & Markham, the Harper Collins Dictionary of Biology, Harper Perennial, N.Y. (1991) and the like.

As used herein and in the appended claims, the singular forms “a”, “an”, and “the” include plural referents unless the context clearly dictates otherwise. For example, the term “a protein” refers to one or more proteins, i.e., a single protein and multiple proteins. The claims can be drafted to exclude any optional element when exclusive terminology is used such as “solely,” “only” are used in connection with the recitation of claim elements or when a negative limitation is specified.

Aspects of the present disclosure can be further understood in light of the embodiments, section headings, figures, descriptions and examples, none of which should be construed as limiting the entire scope of the present disclosure in any way. Accordingly, the claims set forth below should be construed in view of the full breadth and spirit of the disclosure.

Each of the individual embodiments described and illustrated herein has discrete components and features which may be readily separated from or combined with the features of any of the other several embodiments without departing from the scope or spirit of the present teachings. Any recited method can be carried out in the order of events recited or in any other order which is logically possible.

Numeric ranges are inclusive of the numbers defining the range. All numbers should be understood to encompass the midpoint of the integer above and below the integer i.e. the number 2 encompasses 1.5-2.5. The number 2.5 encompasses 2.45-2.55 etc. When sample numerical values are provided, each alone may represent an intermediate value in a range of values and together may represent the extremes of a range unless specified.

In the context of the present disclosure, “non-naturally occurring” refers to a polynucleotide, polypeptide, carbohydrate, lipid, or composition that does not exist in nature. Such a polynucleotide, polypeptide, carbohydrate, lipid, or composition may differ from naturally occurring polynucleotides polypeptides, carbohydrates, lipids, or compositions in one or more respects. For example, a polymer (e.g., a polynucleotide, polypeptide, or carbohydrate) may differ in the kind and arrangement of the component building blocks (e.g., nucleotide sequence, amino acid sequence, or sugar molecules). A polymer may differ from a naturally occurring polymer with respect to the molecule(s) to which it is linked. For example, a “non-naturally occurring” protein may differ from naturally occurring proteins in its secondary, tertiary, or quaternary structure, by having a chemical bond (e.g., a covalent bond including a peptide bond, a phosphate bond, a disulfide bond, an ester bond, and ether bond, and others) to a polypeptide (e.g., a fusion protein), a lipid, a carbohydrate, or any other molecule. Similarly, a “non-naturally occurring” polynucleotide or nucleic acid may contain one or more other modifications (e.g., an added label or other moiety) to the 5′-end, the 3′ end, and/or between the 5′- and 3′-ends (e.g., methylation) of the nucleic acid. A “non-naturally occurring” composition may differ from naturally occurring compositions in one or more of the following respects: (a) having components that are not combined in nature, (b) having components in concentrations not found in nature, (c) omitting one or components otherwise found in naturally occurring compositions, (d) having a form not found in nature, e.g., dried, freeze dried, crystalline, aqueous, and (e) having one or more additional components beyond those found in nature (e.g., buffering agents, a detergent, a dye, a solvent or a preservative). All publications, patents, and patent applications mentioned in this specification are herein incorporated by reference to the same extent as if each individual publication, patent, or patent application was specifically and individually indicated to be incorporated by reference.

Embodiments include the following:
1. A synthetic nucleic acid oligonucleotide, comprising (a) one or more tags with a sequence that is not contained in a eukaryotic immune repertoire wherein at least one tag is positioned between a region corresponding to a diversity and joining region ((D)J) and a constant region (C) of an immunoglobulin (IG) or T cell receptor thereby distinguishing synthetic oligonucleotides from naturally occurring sequences in the eukaryotic immune repertoire; and wherein the V(D)J region has a predetermined V(D)J clonotype, the synthetic oligonucleotide optionally further comprising a tag proximate to a region corresponding to a eukaryotic variable region (V); and optionally wherein C further comprises one or more priming sites for nucleic acid amplification.
2. The synthetic oligonucleotide according to embodiment 1, wherein the oligonucleotide is a single strand DNA or a double-stranded DNA further comprising a promoter sequence for a DNA dependent RNA polymerase.
3. The synthetic oligonucleotide according to embodiment 2, wherein one of the tags is positioned between the T7 promoter and the variable region.
4. The synthetic oligonucleotide of embodiments 1 or 2, comprising a length in the range of 200-1000 nucleotides for example in the range of 450-700 nucleotides.
5. The synthetic oligonucleotide of any previous embodiment, wherein the tag comprises a number of nucleotides in the range of 6-25 nucleotides e.g. 8 nucleotides.
6. The synthetic oligonucleotide of any previous embodiment, wherein if the synthetic oligonucleotide is a double-stranded (dsDNA) or single-stranded DNA (ssDNA) oligonucleotide, the oligonucleotide further comprises a poly-dT tail.
7. The synthetic oligonucleotide of any previous embodiment wherein if the synthetic oligonucleotide is an RNA oligonucleotide, the oligonucleotide further comprises a poly-A tail.
8. The synthetic oligonucleotide of any previous embodiment, wherein the synthetic DNA oligonucleotide is inserted into a bacterial plasmid, is a linear dsDNA or has a priming site for nucleic acid amplification.
9. The synthetic oligonucleotide of any previous embodiment, further comprising sequencing adaptors.
10. The synthetic oligonucleotide of any previous embodiment, wherein the eukaryotic immune repertoire is a mammalian immune repertoire.
11. The synthetic oligonucleotide according to any previous embodiment, wherein the synthetic oligonucleotide is contained within a mixture of synthetic oligonucleotides wherein each oligonucleotide in the mixture contains a different V(D)J clonotype.
12. The synthetic oligonucleotide according to embodiment 11, wherein the mixture of clonotypes comprises each of at least 2 e.g. 5 different clonotypes.
13. The synthetic oligonucleotide according to embodiment 12, wherein a plurality of mixtures of clonotypes are combined to form an internal control for a eukaryotic derived sample.
14. The synthetic oligonucleotide according to embodiment 13, wherein the plurality of mixtures of clonotypes comprises at least 2 mixtures e.g. 10 mixtures for forming a control pool.
15. A method for RNA analysis of an immune response, comprising:
   (a) obtaining a synthetic oligonucleotide according to any of embodiments 1-14;
   (b) optionally performing in vitro transcription of the synthetic oligonucleotide if the synthetic oligonucleotide is DNA and omitting this step if the synthetic oligonucleotide is RNA;
   (c) forming a control pool of RNA oligonucleotides from the DNA oligonucleotides in (b) or from the RNA oligonucleotides in (a) wherein the control pool of RNA oligonucleotides comprises a plurality of mixtures of clonotypes wherein each mixture comprises a plurality of clonotypes at different predetermined concentrations;
   (d) adding a predetermined concentration of the control pool of RNA from (c) to a sample obtained from a eukaryote RNAs produced by an immune response; and
   (e) preparing a library of (d) for sequencing.
16. The method according to embodiment 15, wherein (a) further comprises adding a polynucleotide tail to the synthetic oligonucleotide by means of a primer and amplifying the primed oligonucleotide.
17. The method according to embodiments 15 or 16, wherein (d) further comprises sequencing the RNA from (e) to analyze the RNA associated with the immune response.
18. The method according to any of claims 15-17, further comprising identifying the control pool of the synthetic oligonucleotides.
19. The method according to any of embodiments 15-18, further comprising using the identified control pool for quantifying, normalizing, and detecting the sensitivity and accuracy of the eukaryotic RNA associated with the immune response.

TABLE 2
Natural V(D)J combination

| | SEQ ID | | SEQ ID | |
|---|---|---|---|---|
| Name | NO | DNA sequence | NO | Transcribed RNA sequence |
| Human- | 1 | CAATCCGCCCTCACTACAACCGGATACGCCGGTCTCGAATGTAATAC | 2 | GGCACGTAGTTCCGGAGCTTCCACCACCGACAGCTCTCTGAGGCCAAGGGA |
| IGHA_0 | | GACTCACTATAGGCACGTAGTTCCGGAGCTTCCACCACCGACAGCTC | | CAGCTTTCGCGGCTCCCGCTCGCTCAGCTTTCGGATGCGGGAGCCCCTGTCC |
| | | TCTGAGGCCAAGGGACAGCTTTCGCGGCTCCCGCTCGCTCAGCTTTC | | AGCATCTCCAGCGTGCGGAGCATCTGAAGTTGCAGTCTTGCGTGTGGATGG |
| | | GGATGCGGGAGCCCCTGTCCAGCATCTCCAGCGTGCGGAGCATCTG | | TGCAGCCACCGGGTGCGTGCCAGGCAGGCCCTCCTGGGGTACAGGAAGCT |
| | | AAGTTGCAGTCTTGCGTGTGGATGGTGCAGCCACCGGGTGCGTGCC | | GTGTGCACGCAGCCTCGCCTGTATGGGGAGCAGGGAACGGGACAGGCCCC |
| | | AGGCAGGCCCTCCTGGGGTACAGGAAGCTGTGTGCACGCAGCCTCG | | CATGGTCTTCCCGGTGGCCTCTCGGGGCTTCTGACGCCAAATGGGCTTCCCA |
| | | CCTGTATGGGGAGCAGGGAACGGGACAGGCCCCCATGGTCTTCCCG | | TGGTCACCCTGGACAAGGAGGCAACCACCCCACCTCCACGTAGTGCATCCCC |
| | | GTGGCCTCTCGGGGCTTCTGACGCCAAATGGGCTTCCCATGGTCACC | | GACCAGCCCCAAGGTCTTCCCGCTGAGCCTCTGCAGCACCCCCCAAGATGGG |
| | | CTGGACAAGGAGGCAACCACCCCACCTCCACGTAGTGCATCCCCGAC | | AACGTGGTCGTCGCATGCCTGGTCCAGGGCTTCTTCCCCCAGAAGAGCGAT C |
| | | CAGCCCCAAGGTCTTCCCGCTGAGCCTCTGCAGCACCCCCCAAGATG | | |
| | | GGAACGTGGTCGTCGCATGCCTGGTCCAGGGCTTCTTCCCCCAGAAG | | |
| | | AGCGATCATTCGGAGACCTCCCGCTACTCTGGCGTCGATGAGGGA | | |
| Human- | 3 | CAATCCGCCCTCACTACAACCGGATACGCCGGTCTCGAATGTAATAC | 4 | GGCACGTAGTAGGAGGAATGTAAGCTGGAGAGAAATCAGTCTCTAAAAC |
| IGHD_0 | | GACTCACTATAGGCACGTAGTAGGAGGAATGTAAGCTGGAGAGA | | TGAAGAATGACATTGAAAATCGGCCCAAGAAGGAGCAGGTTCTGGAACT |
| | | AATCAGTCTCTAAAACTGAAGAATGACATTGAAAATCGGCCCAAG | | GGAGCGGGAGAATGAAATGCTGAAGACCAAAAACCAGGAGCTGCAGTCC |
| | | AAGGAGCAGGTTCTGGAACTGGAGCGGGAGAATGAAATGCTGAA | | ATCATCCAGGCCGGGAAGCGCAGCCTGCCAGACTCAGACAAGGCCATCCTG |
| | | GACCAAAAACCAGGAGCTGCAGTCCATCATCCAGGCCGGGAAGCG | | GACATCTTGGAACACGACCGCAAGGAGGCCCTGGAGGACAGGCAGGAGC |
| | | CAGCCTGCCAGACTCAGACAAGGCCATCCTGGACATCTTGGAACAC | | TGGTCAACAGGATCTACAACCTGCAGGAGGAGGCCCGCCAGGCAGAGGA |
| | | GACCGCAAGGAGGCCCTGGAGGACAGGCAGGAGCTGGTCAACAG | | GCTGCGAGACAAGGCCTTCCACCACGTAGTGCACCCACCAAGGCTCCGGAT |
| | | GATCTACAACCTGCAGGAGGAGGCCCGCCAGGCAGAGGAGCTGCG | | GTGTTCCCCATCATATCAGGGTGCAGACACCCAAAGGATAACAGCCCTGTG |
| | | AGACAAGGCCTTCCACCACGTAGTGCACCCACCAAGGCTCCGGATG | | GTCCTGGCATGCTTGATAACTGGGTACCAGAAGAGCGATC |
| | | TGTTCCCCATCATATCAGGGTGCAGACACCCAAAGGATAACAGCCC | | |
| | | TGTGGTCCTGGCATGCTTGATAACTGGGTACCAGAAGAGCGATCAT | | |
| | | TCGGAGACCTCCCGCTACTCTGGCGTCGATGAGGGA | | |
| Human- | | CAATCCGCCCTCACTACAACCGGATACGCCGGTCTCGAATGTAATAC | 5 | GGCACGTAGTCGATCTTTGCAGCAGCGGGTCAAGGGGAAGACGGATGGG |
| IGHE_2 | | GACTCACTATAGGCACGTAGTCGATCTTTGCAGCAGCGGGTCAAGG | | CTCTGTGTGGAGGCTGAAGCGCCGGGTCGTCCATTTGGTGCCTGTGGATGG |
| | | GGAAGACGGATGGGCTCTGTGTGGAGGCTGAAGCGCCGGGTCGTC | | TGTGGCCCGTCCGGCTCCCTGTCGGGTTCCTGGACAGCTCCCAGATGATCAG |
| | | CATTTGGTGCCTGTGGATGGTGTGGCCCGTCCGGCTCCCTGTCGGGT | | TAACCGTGGTTGTTATTTCTGTGCCGGGCAGTGGAGCCTGGGTAGGGGGA |
| | | TCCTGGACAGCTCCCAGATGATCAGTAACCGTGGTTGTTATTTCTGT | | GCTCTGCCTCAGTGCTTTCAGCTAAAAATGGGGTGGGAACCCCCGGAGGCC |
| | | GCCGGGCAGTGGAGCCTGGGTAGGGGGAGCTCTGCCTCAGTGCTTT | | CGGGCCGCCCTGGAAGTTCCCTTTTCTCTCTCCCCGTAGATTTTATTAATAAT |
| | | CAGCTAAAAATGGGGTGGGAACCCCCGGAGGCCCGGGCCGCCCTG | | CCATAGATCGCACGTAGTGCCTCCACACAGAGCCCATCCGTCTTCCCCTTGA |
| | | GAAGTTCCCTTTTCTCTCTCCCCGTAGATTTTATTAATAATCCATAGA | | CCCGCTGCTGCAAAAACATTCCCTCCAATGCCACCTCCGTGACTCTGGGCTG |
| | | TCGCACGTAGTGCCTCCACACAGAGCCCATCCGTCTTCCCCTTGACCC | | CCTGGCCACGGGCTAGAAGAGCGATC |
| | | GCTGCTGCAAAAACATTCCCTCCAATGCCACCTCCGTGACTCTGGGC | | |
| | | TGCCTGGCCACGGGCTAGAAGAGCGATCATTCGGAGACCTCCCGCT | | |
| | | ACTCTGGCGTCGATGAGGGA | | |
| Human- | 5 | CAATCCGCCCTCACTACAACCGGATACGCCGGTCTCGAATGTAATAC | 6 | GGCACGTAGTTCAGGTCCTCTGGTCAATATCTGATGGGATGGTCCACATTT |
| IGHG_0 | | GACTCACTATAGGCACGTAGTTCAGGTCCTCTGGTCAATATCTGATG | | CCTCCTCCCCTGGGGCAGGAGGGTGTGGTAGCTTGTCCAGGATGGCTGAAA |
| | | GGATGGTCCACATTTCCTCCTCCCCTGGGGCAGGAGGGTGTGGTAG | | TCCCATGGAAGTTGATGGTCATGGGATTAACCCTTTCTTCTCTCAGAGTGTC |
| | | CTTGTCCAGGATGGCTGAAATCCCATGGAAGTTGATGGTCATGGGA | | CAGCAACCCTGCCTGGCCAGGATTGGAATCCGTCCACAGTTAAGCTTTGCA |
| | | TTAACCCTTTCTTCTCTCAGAGTGTCCAGCAACCCTGCCTGGCCAGGA | | TTCTCAAAAGTAGTACAAGCTAGACCACTGAGTATGAATGAACCACAAAG |
| | | TTGGAATCCGTCCACAGTTAAGCTTTGCATTCTCAAAAGTAGTACA | | TCCAAGGGCCATTTCAGTAGAAAGCTCACACTGCAGAGCTCTGGACCTTTT |
| | | AGCTAGACCACTGAGTATGAATGAACCACAAAGTCCAAGGGCCAT | | CACATGCTCCAGGCACTGTCCTCAGGATGTTTTTCCACGTAGTGCCTCCACC |
| | | TTCAGTAGAAAGCTCACACTGCAGAGCTCTGGACCTTTTCACATGCT | | AAGGGCCCATCGGTCTTCCCCCTGGTGCCCTCCTCCAGGAGCGTCTCTGAGG |
| | | CCAGGCACTGTCCTCAGGATGTTTTTCCACGTAGTGCCTCCACCAAG | | GCACAGCGGCCCTGGGCTGCCTGGTCAAGGACTACTTCCAGAAGAGCGATC |
| | | GGCCCATCGGTCTTCCCCCTGGTGCCCTCCTCCAGGAGCGTCTCTGAG | | |
| | | GGCACAGCGGCCCTGGGCTGCCTGGTCAAGGACTACTTCCAGAAGA | | |
| | | GCGATCATTCGGAGACCTCCCGCTACTCTGGCGTCGATGAGGGA | | |
| Human- | 7 | CAATCCGCCCTCACTACAACCGGATACGCCGGTCTCGAATGTAATAC | 8 | GGCACGTAGTTTCGTTTGATCTCCAGCTTGGTCCCCTGGCCAAAAGTGTACG |
| IGK_0 | | GACTCACTATAGGCACGTAGTTTCGTTTGATCTCCAGCTTGGTCCCCT | | GGGTACTGTAACTCTGTTGACAGTAGTAAGTTGCAAAATCTTCAGGTTGCA |
| | | GGCCAAAAGTGTACGGGGTACTGTAACTCTGTTGACAGTAGTAAG | | GACTGCTGATGGTGAGAGTGAAATCTGTCCCAGATCCACTGCCACTGAACC |
| | | TTGCAAAATCTTCAGGTTGCAGACTGCTGATGGTGAGAGTGAAATC | | TTGATGGGACCCCACTTTGCAAACTGGATGCAGCATAGATCAGGAGCTTA |
| | | TGTCCCAGATCCACTGCCACTGAACCTTGATGGGACCCCACTTTGCA | | GGGGCTTTCCCTGGTTTCTGCTGATACCAATTTAAATAGCTGCTAATGCTCT |
| | | AACTGGATGCAGCATAGATCAGGAGCTTAGGGGCTTTCCCTGGTTT | | GACTTGCCCGGCAAGTGATGGTGACTCTGTCTCCTACAGATGCAGACAGGG |
| | | CTGCTGATACCAATTTAAATAGCTGCTAATGCTCTGACTTGCCCGGC | | AGGATGGAGACTGGGTCATCTGGATGTCACATCTGGCACCTCGGAGCCAG |
| | | AAGTGATGGTGACTCTGTCTCCTACAGATGCAGACAGGGAGGATG | | AGTAGCAGGAGCCCCAGGAGCTGAGCGGGGACCCTCATGTCCATGCTGTG |
| | | GAGACTGGGTCATCTGGATGTCACATCTGGCACCTCGGAGCCAGAG | | TCCTGACTGAGACTCCACGTAGTCGAACTGTGGCTGCACCATCTGTCTTCAT |
| | | TAGCAGGAGCCCCAGGAGCTGAGCGGGGACCCTCATGTCCATGCTG | | CTTCCCGCCATCTGATGAGCAGTTGAAATCTGGAACTGCCTCTGTTGTGTGA |
| | | TGTCCTGACTGAGACTCCACGTAGTCGAACTGTGGCTGCACCATCTG | | GAAGAGCGATO |
| | | TCTTCATCTTCCCGCCATCTGATGAGCAGTTGAAATCTGGAACTGCC | | |
| | | TCTGTTGTGTGAGAAGAGCGATCATTCGGAGACCTCCCGCTACTCTG | | |
| | | GCGTCGATGAGGGA | | |
| Human- | 9 | CAATCCGCCCTCACTACAACCGGATACGCCGGTCTCGAATGTAATAC | 10 | GGCACGTAGTCGAGGGGGCAGCCTTGGGCTGACCTAGGACGGTCAGCTTG |
| IGL_0 | | GACTCACTATAGGCACGTAGTCGAGGGGGCAGCCTTGGGCTGACCT | | GTCCCTCCGCCGAATACCACAGTGCTGCTGTCCCACGCCTGACAGTAATAGT |
| | | AGGACGGTCAGCTTGGTCCCTCCGCCGAATACCACAGTGCTGCTGTC | | CAGCCTCATCCATAGCCTGGGTCCCGCTGATGGTCAGAGTGGCTGTGTTCCC |
| | | CCACGCCTGACAGTAATAGTCAGCCTCATCCATAGCCTGGGTCCCGC | | AGAGTTGGAGCCAGAGAATCGCTCAGGGATCCCTGAGGGCCGCTTGCTAT |
| | | TGATGGTCAGAGTGGCTGTGTTCCCAGAGTTGGAGCCAGAGAATC | | CTTGATAGATGACCAGCACAGGGGACTGGCCTGGCTTCTGCTGATACCAGC |
| | | GCTCAGGGATCCCTGAGGGCCGCTTGCTATCTTGATAGATGACCAG | | AAGCATATTTATCCCCCAATTTATCTCCAGAGCAGGTGATGCTGGCTGTCTG |
| | | CACAGGGGACTGGCCTGGCTTCTGCTGATACCAGCAAGCATATTTA | | TCCTGGGGACACGGACACTGAGGGTGGCTGAGTCAGCTCATAGGAGGCCA |
| | | TCCCCCAATTTATCTCCAGAGCAGGTGATGCTGGCTGTCTGTCCTGG | | CGGATCCTGTGCAGTAAGCAAGGACGCCGAGGAAGAGAGGGATCCATGC |
| | | GGACACGGACACTGAGGGTGGCTGAGTCAGCTCATAGGAGGCCAC | | CATGGCTGAGGCACCTCCGATGCTGCTTCCTGAGGCCCCCACGTAGTGGTCA |
| | | GGATCCTGTGCAGTAAGCAAGGACGCCGAGGAAGAGAGGGATCC | | GCCCAAGGCTGCCCCCTCGGTCACTCTGTTCCCGCCCTCCTCTGAGGAGCTTC |
| | | ATGCCATGGCTGAGGCACCTCCGATGCTGCTTCCTGAGGCCCCCACG | | AAGCCAACAAGGCCACACTGGTGTGTCTCATAAGAAGAGCGATC |
| | | TAGTGGTCAGCCCAAGGCTGCCCCCTCGGTCACTCTGTTCCCGCCCTC | | |
| | | CTCTGAGGAGCTTCAAGCCAACAAGGCCACACTGGTGTGTCTCATA | | |
| | | AGAAGAGCGATCATTCGGAGACCTCCCGCTACTCTGGCGTCGATGA | | |
| | | GGGA | | |
| Human- | 11 | CAATCCGCCCTCACTACAACCGGATACGCCGGTCTCGAATGTAATAC | 12 | GGCACGTAGTCATCCAAGCGAGCACCTGCTTCCTATTTTCCGACTAATTTCG |
| TRA_0 | | GACTCACTATAGGCACGTAGTCATCCAAGCGAGCACCTGCTTCCTAT | | GTCCCCTGATACGGAATCCGGGATCCTGCAGACAGGCAGCGGGTGACAGC |
| | | TTTCCGACTAATTTCGGTCCCCTGATACGGAATCCGGGATCCTGCAG | | AAGCACAAGGGTGTTCGATGACACCTGGAGAGGTCAGGAGAGCCAGTCCT |
| | | ACAGGCAGCGGGTGACAGCAAGCACAAGGGTGTTCGATGACACCT | | GCGGTGCAGAGGAAGAAACCCAGCACAGAGGCCAACAGAGCCAGCCCCA |
| | | GGAGAGGTCAGGAGAGCCAGTCCTGCGGTGCAGAGGAAGAAACC | | GCCTCTGGAGGCCTGAAGGTACCGCCCTGCCGCGCGTGGGCACTGCTCCCTG |
| | | CAGCACAGAGGCCAACAGAGCCAGCCCCAGCCTCTGGAGGCCTGAA | | GCGCCGTCCGTCCACTGACCATCGTCCCTCGGCCCGGCCGCCCGCGCGGGGC |
| | | GGTACCGCCCTGCCGCGCGTGGGCACTGCTCCCTGGCGCCGTCCGTCC | | AGGCAACGCCCGACCGGGGGCCTCGGGGACCAGGAGGAACCTCCACGTAG |
| | | ACTGACCATCGTCCCTCGGCCCGGCCGCCCGCGCGGGGCAGGCAACG | | TATATCCAGAACCCTGACCCTGCCGTGTACCAGCTGAGAGACTCTAAATCC |
| | | CCCGACCGGGGGCCTCGGGGACCAGGAGGAACCTCCACGTAGTATA | | AGTGACAAGTCTGTCTGCCTATTCACCGATTTTGATTAGAAGAGCGATC |
| | | TCCAGAACCCTGACCCTGCCGTGTACCAGCTGAGAGACTCTAAATCC | | |
| | | AGTGACAAGTCTGTCTGCCTATTCACCGATTTTGATTAGAAGAGCG | | |
| | | ATCATTCGGAGACCTCCCGCTACTCTGGCGTCGATGAGGGA | | |
| Human- | 13 | CAATCCGCCCTCACTACAACCGGATACGCCGGTCTCGAATGTAATAC | 14 | GGCACGTAGTCTTGTTCAGGTCCTCTGGTCAATATCTGATGGGATGGTCCA |
| TRB_0 | | GACTCACTATAGGCACGTAGTCTTGTTCAGGTCCTCTGGTCAATATC | | CATTTCCTCCTCCCCTGGGGCAGGAGGGTGTGGTAGCTTGTCCAGGATGGC |
| | | TGATGGGATGGTCCACATTTCCTCCTCCCCTGGGGCAGGAGGGTGT | | TGAAATCCCATGGAAGTTGATGGTCATGGGATTAACCCTTTCTTCTCTCAG |
| | | GGTAGCTTGTCCAGGATGGCTGAAATCCCATGGAAGTTGATGGTCA | | AGTGTCCAGCAACCCTGCCTGGCCAGGATTGGAATCCGTCCACAGTTAAGC |
| | | TGGGATTAACCCTTTCTTCTCTCAGAGTGTCCAGCAACCCTGCCTGGC | | TTTGCATTCTCAAAAGTAGTACAAGCTAGACCACTGAGTATGAATGAACC |
| | | CAGGATTGGAATCCGTCCACAGTTAAGCTTTGCATTCTCAAAAGTA | | ACAAAGTCCAAGGGCCATTTCAGTAGAAAGCTCACACTGCAGAGCTCTGG |
| | | GTACAAGCTAGACCACTGAGTATGAATGAACCACAAAGTCCAAGG | | ACCTTTTCACATGCTCCAGGCACTGTCCTCAGGATGTTTTGAGCTCCTTAGA |
| | | GCCATTTCAGTAGAAAGCTCACACTGCAGAGCTCTGGACCTTTTCAC | | GCTGGATGTCTTTGTAAGGCTGTTTGGCGAAGGGGCTGGTCTTCTCCAGTA |
| | | ATGCTCCAGGCACTGTCCTCAGGATGTTTTGAGCTCCTTAGAGCTGG | | CCCACCCCTCTCCCCACGTAGTGAGGACCTGAACAAGGTGTTCCCACCCGAG |
| | | ATGTCTTTGTAAGGCTGTTTGGCGAAGGGGCTGGTCTTCTCCAGTAC | | GTCGCTGTGTTTGAGCCATCAGAAGCAGAGATCTCCCACACCCAAAAGGCC |
| | | CCACCCCTCTCCCCACGTAGTGAGGACCTGAACAAGGTGTTCCCACC | | ACACTGGTGAGAAGAGCGATC |
| | | CGAGGTCGCTGTGTTTGAGCCATCAGAAGCAGAGATCTCCCACACC | | |
| | | CAAAAGGCCACACTGGTGAGAAGAGCGATCATTCGGAGACCTCCC | | |
| | | GCTACTCTGGCGTCGATGAGGGA | | |
| Human- | 15 | CAATCCGCCCTCACTACAACCGGATACGCCGGTCTCGAATGTAATAC | 16 | GGCACGTAGTTGTGACAAAGTAACCCAGAGTTCCCCGGACCAGACGGTGG |
| TRD_0 | | GACTCACTATAGGCACGTAGTTGTGACAAAGTAACCCAGAGTTCCC | | CGAGTGGCAGTGAGGTGGTACTGCTCTGCACTTACGACACTGTATATTCAA |
| | | CGGACCAGACGGTGGCGAGTGGCAGTGAGGTGGTACTGCTCTGCA | | ATCCAGATTTATTCTGGTACCGGATAAGGCCAGATTATTCCTTTCAGTTTGT |
| | | CTTACGACACTGTATATTCAAATCCAGATTTATTCTGGTACCGGATA | | CTTTTATGGGGATAACAGCAGATCAGAAGGTGCAGATTTTACTCAAGGAC |
| | | AGGCCAGATTATTCCTTTCAGTTTGTCTITTATGGGGATAACAGCAG | | GGTTTTCTGTGAAACACATTCTGACCCAGAAAGCCTTTCACTTGGTGATCTC |
| | | ATCAGAAGGTGCAGATTTTACTCAAGGACGGTTTTCTGTGAAACAC | | TCCAGTAAGGACTGAAGACAGTGCCACTTACTACTGTGCCTTTAGCCAGAC |
| | | ATTCTGACCCAGAAAGCCTTTCACTTGGTGATCTCTCCAGTAAGGAC | | CCCTGATCTTTGGCAAAGGAACCTATCTGGAGGTACAACAACCACGTAGTG |
| | | TGAAGACAGTGCCACTTACTACTGTGCCTTTAGCCAGACCCCTGATC | | AAGTCAGCCTCATACCAAACCATCCGTTTTTGTCATGAAAAATGGAACAAA |
| | | TTTGGCAAAGGAACCTATCTGGAGGTACAACAACCACGTAGTGAA | | TGTCGCTTGTCTGGTGAAGGAATTCTACCCCAAGGAAGAAGAGCGATC |
| | | GTCAGCCTCATACCAAACCATCCGTTTTTGTCATGAAAAATGGAAC | | |
| | | AAATGTCGCTTGTCTGGTGAAGGAATTCTACCCCAAGGAAGAAGA | | |
| | | GCGATCATTCGGAGACCTCCCGCTACTCTGGCGTCGATGAGGGA | | |
| Human- | 17 | CAATCCGCCCTCACTACAACCGGATACGCCGGTCTCGAATGTAATAC | 18 | GGCACGTAGTGCAGGTCACCTAGAGCAACCTCAAATTTCCAGTACTAAAAC |
| TRG_0 | | GACTCACTATAGGCACGTAGTGCAGGTCACCTAGAGCAACCTCAAA | | GCTGTCAAAAACAGCCCGCCTGGAATGTGTGGTGTCTGGAATAAAAATTT |
| | | TTTCCAGTACTAAAACGCTGTCAAAAACAGCCCGCCTGGAATGTGT | | CTGCAACATCTGTATATTGGTATCGAGAGAGTCCTGGTGAAGTCATACAGT |
| | | GGTGTCTGGAATAAAAATTTCTGCAACATCTGTATATTGGTATCGA | | TCCTGGTGTCCATTTCATATGACGGCACTGTCAGAAAGGAATCTGGCATTC |
| | | GAGAGTCCTGGTGAAGTCATACAGTTCCTGGTGTCCATTTCATATG | | CGTCAGGCAAATTTGAGGTGGATAGGATACCTGAAACGTCTACATCCACTC |
| | | ACGGCACTGTCAGAAAGGAATCTGGCATTCCGTCAGGCAAATTTGA | | TCACCATTCACAATGTAGAGAAACAGGACATAGCTACCTACTACTGTGCCT |
| | | GGTGGATAGGATACCTGAAACGTCTACATCCACTCTCACCATTCACA | | TGTGGGAGGTGATAGTAGTGATTGGATCAAGACGTTTGCAAAAGGGACT |
| | | ATGTAGAGAAACAGGACATAGCTACCTACTACTGTGCCTTGTGGGA | | AGGCTCATAGTAACTTCGCCTGCACGTAGTGATAAACAACTTGATGCAGAT |
| | | GGTGATAGTAGTGATTGGATCAAGACGTTTGCAAAAGGGACTAGG | | GTTTCCCCCAAGCCCACTATTTTTCTTCCTTCGATTGCTGAAACAAAACTCCA |
| | | CTCATAGTAACTTCGCCTGCACGTAGTGATAAACAACTTGATGCAG | | GAAGGCTGGAACATACAGAAGAGCGATC |
| | | ATGTTTCCCCCAAGCCCACTATTTTTCTTCCTTCGATTGCTGAAACAA | | |
| | | AACTCCAGAAGGCTGGAACATACAGAAGAGCGATCATTCGGAGAC | | |
| | | CTCCCGCTACTCTGGCGTCGATGAGGGA | | |
| Human- | 19 | CAATCCGCCCTCACTACAACCGGATACGCCGGTCTCGAATGTAATAC | 20 | GGCACGTAGTCAGGTCACCTTGAGGGAGTCTGGTCCTGCGCTGGTGAAACC |
| IGHM3 | | GACTCACTATAGGCACGTAGTCAGGTCACCTTGAGGGAGTCTGGTC | | CACACAGACCCTCACACTGACCTGCACCTTCTCTGGGTTCTCACTCAGCACTA |
| | | CTGCGCTGGTGAAACCCACACAGACCCTCACACTGACCTGCACCTTC | | GTGGAATGTGTGTGAGCTGGGTCCGTCAGCCCCCAGGGAAGGCCCTGGAG |
| | | TCTGGGTTCTCACTCAGCACTAGTGGAATGTGTGTGAGCTGGGTCC | | TGGCTTGCACTCATTGATTGGGATGATGATAAATACTACAGCACATCTCTG |
| | | GTCAGCCCCCAGGGAAGGCCCTGGAGTGGCTTGCACTCATTGATTG | | AAGACCAGGCTCACCATCTCCAAGGACACCTCCAAAAACCAGGTGGTCCTT |
| | | GGATGATGATAAATACTACAGCACATCTCTGAAGACCAGGCTCACC | | ACAATGACCAACATGGACCCTGTGGACACAGCCACGTATTACTGTGCACGG |
| | | ATCTCCAAGGACACCTCCAAAAACCAGGTGGTCCTTACAATGACCA | | ATACAAGCGACGCGCAGTGGATACAGCTATGGTTACACGACAACTGGTTC |
| | | ACATGGACCCTGTGGACACAGCCACGTATTACTGTGCACGGATACA | | GACCCCTGGGGCCAGGGAACCCTGGTCACCGTCTCCTCAGCACGTAGTGGG |
| | | AGCGACGCGCAGTGGATACAGCTATGGTTACACGACAACTGGTTCG | | AGTGCATCCGCCCCAACCCTTTTCCCCCTCGTCTCCTGTGAGAATTCCCCGTC |
| | | ACCCCTGGGGCCAGGGAACCCTGGTCACCGTCTCCTCAGCACGTAGT | | GGATACGAGCAGCGTGGCCGTTGGCTGCCTCGCACAGGACTTCC |
| | | GGGAGTGCATCCGCCCCAACCCTTTTCCCCCTCGTCTCCTGTGAGAAT | | TCCCCGTCGGATACGAGCAGCGTGGCCGTTGGCTGCCTCGCACAGG |
| | | ACTTCCATTCGGAGACCTCCCGCTACTCTGGCGTCGATGAGGGA | | |
| Human- | 21 | CAATCCGCCCTCACTACAACCGGATACGCCGGTCTCGAATGTAATAC | 22 | GGCACGTAGTCAGGTCACCTTGAAGGAGTCTGGTCCTGCGCTGGTGAAACC |
| IGHM4 | | GACTCACTATAGGCACGTAGTCAGGTCACCTTGAAGGAGTCTGGTC | | CACACAGACCCTCACACTGACCTGCACCTTCTCTGGGTTCTCACTCAGCACTA |
| | | CTGCGCTGGTGAAACCCACACAGACCCTCACACTGACCTGCACCTTC | | GTGGAATGCGTGCGAGCTGGATCCGTCAGCCCCCAGGGAAGGCCCTGGAG |
| | | TCTGGGTTCTCACTCAGCACTAGTGGAATGCGTGCGAGCTGGATCC | | TGGCTTGCACGCATTGATTGGGATGATGATAAATTCTACAGCACATCTCTG |
| | | GTCAGCCCCCAGGGAAGGCCCTGGAGTGGCTTGCACGCATTGATTG | | AAGACCAGGCTCACCATCTCCAAGGACACCTCCAAAAACCAGGTGGTCCTT |
| | | GGATGATGATAAATTCTACAGCACATCTCTGAAGACCAGGCTCACC | | ACAATGACCAACATGGACCCTGTGGACACAGCCACGTATTACTGTGCACGG |
| | | ATCTCCAAGGACACCTCCAAAAACCAGGTGGTCCTTACAATGACCA | | ATACCAGGCGACAACAGACCCGTATTATGATTACATTTGGGGGAGTTATC |
| | | ACATGGACCCTGTGGACACAGCCACGTATTACTGTGCACGGATACC | | GTTATACCACCAGACATTACTACTACTACTACGGTATGGACGTCTGGGGCA |
| | | AGGCGACAACAGACCCGTATTATGATTACATTTGGGGGAGTTATCG | | AAGGGACCACGGTCACCGTCTCCTCAGCACGTAGTGGGAGTGCATCCGCCC |
| | | TTATACCACCAGACATTACTACTACTACTACGGTATGGACGTCTGGG | | CAACCCTTTTCCCCCTCGTCTCCTGTGAGAATTCCCCGTCGGATACGAGCAGC |
| | | GCAAAGGGACCACGGTCACCGTCTCCTCAGCACGTAGTGGGAGTGC | | GTGGCCGTTGGCTGCCTCGCACAGGACTTCC |
| | | ATCCGCCCCAACCCTTTTCCCCCTCGTCTCCTGTGAGAATTCCCCGTCG | | |
| | | GATACGAGCAGCGTGGCCGTTGGCTGCCTCGCACAGGACTTCCATTC | | |
| | | GGAGACCTCCCGCTACTCTGGCGTCGATGAGGGA | | |
| Human- | 23 | CAATCCGCCCTCACTACAACCGGATACGCCGGTCTCGAATGTAATAC | 24 | GGCACGTAGTGAGGTCCAGCTGGTACAGTCTGGGGCTGAGGTGAAGAAG |
| IGHD7 | | GACTCACTATAGGCACGTAGTGAGGTCCAGCTGGTACAGTCTGGGG | | CCTGGGGCTACAGTGAAAATCTCCTGCAAGGTTTCTGGATACACCTTCACC |
| | | CTGAGGTGAAGAAGCCTGGGGCTACAGTGAAAATCTCCTGCAAGG | | GACTACTACATGCACTGGGTGCAACAGGCCCCTGGAAAAGGGCTTGAGTG |
| | | TTTCTGGATACACCTTCACCGACTACTACATGCACTGGGTGCAACAG | | GATGGGACTTGTTGATCCTGAAGATGGTGAAACAATATACGCAGAGAAG |
| | | GCCCCTGGAAAAGGGCTTGAGTGGATGGGACTTGTTGATCCTGAA | | TTCCAGGGCAGAGTCACCATAACCGCGGACACGTCTACAGACACAGCCTAC |
| | | GATGGTGAAACAATATACGCAGAGAAGTTCCAGGGCAGAGTCACC | | ATGGAGCTGAGCAGCCTGAGATCTGAGGACACGGCCGTGTATTACTGTGC |
| | | ATAACCGCGGACACGTCTACAGACACAGCCTACATGGAGCTGAGCA | | AACAGAAGCAAAAGCCCTATGTAACAGACCATGGCCCCATGAAAGCGGA |
| | | GCCTGAGATCTGAGGACACGGCCGTGTATTACTGTGCAACAGAAGC | | GCCCAAACTACTTTGACTACTGGGGCCAAGGAACCCTGGTCACCGTCTCCTC |
| | | AAAAGCCCTATGTAACAGACCATGGCCCCATGAAAGCGGAGCCCA | | AGCACGTAGTGCACCCACCAAGGCTCCGGATGTGTTCCCCATCATATCAGG |
| | | AACTACTTTGACTACTGGGGCCAAGGAACCCTGGTCACCGTCTCCTC | | GTGCAGACACCCAAAGGATAACAGCCCTGTGGTCCTGGCATGCTTGATAAC |
| | | AGCACGTAGTGCACCCACCAAGGCTCCGGATGTGTTCCCCATCATAT | | TGGGTACC |
| | | CAGGGTGCAGACACCCAAAGGATAACAGCCCTGTGGTCCTGGCATG | | |
| | | CTTGATAACTGGGTACCATTCGGAGACCTCCCGCTACTCTGGCGTCG | | |
| | | ATGAGGGA | | |
| Human- | 25 | CAATCCGCCCTCACTACAACCGGATACGCCGGTCTCGAATGTAATAC | 26 | GGCACGTAGTCAGGTGCAGCTGCAGGAGTCGGGCCCAGGACTGGTGAAGC |
| IGHD10 | | GACTCACTATAGGCACGTAGTCAGGTGCAGCTGCAGGAGTCGGGCC | | CTTCACAGACCCTGTCCCTCACCTGCACTGTCTCTGGTGGCTCCATCAGCAGT |
| | | CAGGACTGGTGAAGCCTTCACAGACCCTGTCCCTCACCTGCACTGTC | | GGTAGTTACTACTGGAGCTGGATCCGCCAGCACCCAGGGAAGGGCCTGGA |
| | | TCTGGTGGCTCCATCAGCAGTGGTAGTTACTACTGGAGCTGGATCC | | GTGGATTGGGTACATCTATTACAGTGGGAGCACCTACTACAACCCGTCCCT |
| | | GCCAGCACCCAGGGAAGGGCCTGGAGTGGATTGGGTACATCTATT | | CAAGAGTCGAGTTACCATATCAGTAGACACGTCTAAGAACCAGTTCTCCCT |
| | | ACAGTGGGAGCACCTACTACAACCCGTCCCTCAAGAGTCGAGTTAC | | GAAGCTGAGCTCTGTGACTGCCGCGGACACGGCCGTGTATTACTGACAAG |
| | | CATATCAGTAGACACGTCTAAGAACCAGTTCTCCCTGAAGCTGAGC | | GCACGACCGAGTCTCCAAAGGCACAGGCCTCCTCAGTGCCCACTGCACAAC |
| | | TCTGTGACTGCCGCGGACACGGCCGTGTATTACTGACAAGGCACGA | | CCCAAGCAGAGGGCAGCCTCGCCAAGGCAACCACAGCCCCAGCCACCACCC |
| | | CCGAGTCTCCAAAGGCACAGGCCTCCTCAGTGCCCACTGCACAACCC | | GTAACACAGGGGCGGCTGATGCTTTTGATATCTGGGGCCAAGGGACAATG |
| | | CAAGCAGAGGGCAGCCTCGCCAAGGCAACCACAGCCCCAGCCACCA | | GTCACCGTCTCTTCAGCACGTAGTGCACCCACCAAGGCTCCGGATGTGTTCC |
| | | CCCGTAACACAGGGGCGGCTGATGCTTTTGATATCTGGGGCCAAGG | | CCATCATATCAGGGTGCAGACACCCAAAGGATAACAGCCCTGTGGTCCTGG |
| | | GACAATGGTCACCGTCTCTTCAGCACGTAGTGCACCCACCAAGGCTC | | CATGCTTGATAACTGGGTACC |
| | | CGGATGTGTTCCCCATCATATCAGGGTGCAGACACCCAAAGGATAA | | |
| | | CAGCCCTGTGGTCCTGGCATGCTTGATAACTGGGTACCATTCGGAG | | |
| | | ACCTCCCGCTACTCTGGCGTCGATGAGGGA | | |
| Human- | 27 | CAATCCGCCCTCACTACAACCGGATACGCCGGTCTCGAATGTAATAC | 28 | GGCACGTAGTTCTGGTGGCTCCGTCAGCAGTGGTAGTTACTACTGGAGCTG |
| IGHA7 | | GACTCACTATAGGCACGTAGTTCTGGTGGCTCCGTCAGCAGTGGTA | | GATCCGGCAGCCCCCAGGGAAGGGACTGGAGTGGATTGGGTATATCTATT |
| | | GTTACTACTGGAGCTGGATCCGGCAGCCCCCAGGGAAGGGACTGG | | ACAGTGGGAGCACCAACTACAACCCCTCCCTCAAGAGTCGAGTCACCATAT |
| | | AGTGGATTGGGTATATCTATTACAGTGGGAGCACCAACTACAACCC | | CAGTAGACACGTCCAAGAACCAGTTCTCCCTGAAGCTGAGCTCTGTGACCG |
| | | CTCCCTCAAGAGTCGAGTCACCATATCAGTAGACACGTCCAAGAAC | | CTGCGGACACGGCCGTGTATTACTGTGCGAGACACACCCAATACCTGGCCA |
| | | CAGTTCTCCCTGAAGCTGAGCTCTGTGACCGCTGCGGACACGGCCGT | | TGACCCCCCTGATCCCTCAGAGCAAGGATGAGAACAGCGATGACTACACG |
| | | GTATTACTGTGCGAGACACACCCAATACCTGGCCATGACCCCCCTGA | | ACCTTTGATGATGTGGGCAGCCTGTGGACCACCCTGTCCACGTTTGTGGCCC |
| | | TCCCTCAGAGCAAGGATGAGAACAGCGATGACTACACGACCTTTGA | | TCTTCATCCTCACCCTCCTCTACAGCGGCATTGTCACTTTCATCAAGACAACA |
| | | TGATGTGGGCAGCCTGTGGACCACCCTGTCCACGTTTGTGGCCCTCT | | ACTGGTTCGACTCCTGGGGCCAAGGAACCCTGGTCACCGTCTCCTCAGCACG |
| | | TCATCCTCACCCTCCTCTACAGCGGCATTGTCACTTTCATCAAGACAA | | TAGTGCATCCCCGACCAGCCCCAAGGTCTTCCCGCTGAGCCTCTGCAGCACC |
| | | CAACTGGTTCGACTCCTGGGGCCAAGGAACCCTGGTCACCGTCTCCT | | CCCCAAGATGGGAACGTGGTCGTCGCATGCCTGGTCCAGGGCTTCTTCCCCC |
| | | CAGCACGTAGTGCATCCCCGACCAGCCCCAAGGTCTTCCCGCTGAGC | | |
| | | CTCTGCAGCACCCCCCAAGATGGGAACGTGGTCGTCGCATGCCTGGT | | |
| | | CCAGGGCTTCTTCCCCCATTCGGAGACCTCCCGCTACTCTGGCGTCGA | | |
| | | TGAGGGA | | |
| Human- | 29 | CAATCCGCCCTCACTACAACCGGATACGCCGGTCTCGAATGTAATAC | 30 | GGCACGTAGTCAGATCACCTTGAAGGAGTCTGGTCCTACGCTGGTGAAACC |
| IGHA3 | | GACTCACTATAGGCACGTAGTCAGATCACCTTGAAGGAGTCTGGTC | | CACACAGACCCTCACGCTGACCCGCACCTTCTCTGGGTTCTCACTCAGCACTA |
| | | CTACGCTGGTGAAACCCACACAGACCCTCACGCTGACCCGCACCTTC | | GTGGAATGTGTGTGAGCTGGATCCGTCAGCCCCCAGGGAAGGCCCTGGAG |
| | | TCTGGGTTCTCACTCAGCACTAGTGGAATGTGTGTGAGCTGGATCC | | TGGCTTGCACTCATTGATTGGGATGATGATAAATACTACAGCACATCTCTG |
| | | GTCAGCCCCCAGGGAAGGCCCTGGAGTGGCTTGCACTCATTGATTG | | AACACCAGGCTCACCATCTCCAAGGACACCTCCAAAAACCAGGTGGTCCTT |
| | | GGATGATGATAAATACTACAGCACATCTCTGAACACCAGGCTCACC | | ACAATGACCAACATGGACCCTGTGGACACAGGCACATATTACTGTGTACG |
| | | ATCTCCAAGGACACCTCCAAAAACCAGGTGGTCCTTACAATGACCA | | GAGCGCAAACACCGAGGTATTAGCATTTTTGGAGTGGTTATTATACCCAAC |
| | | ACATGGACCCTGTGGACACAGGCACATATTACTGTGTACGGAGCGC | | ACAACGCAACAACTGGTTCGACCCCTGGGGCCAGGGAACCCTGGTCACCGT |
| | | AAACACCGAGGTATTAGCATTTTTGGAGTGGTTATTATACCCAACA | | CTCCTCAGCACGTAGTGCATCCCCGACCAGCCCCAAGGTCTTCCCGCTGAGC |
| | | CAACGCAACAACTGGTTCGACCCCTGGGGCCAGGGAACCCTGGTCA | | CTCTGCAGCACCCCCCAAGATGGGAACGTGGTCGTCGCATGCCTGGTCCAG |
| | | CCGTCTCCTCAGCACGTAGTGCATCCCCGACCAGCCCCAAGGTCTTCC | | GGCTTCTTCCCCC |
| | | CGCTGAGCCTCTGCAGCACCCCCCAAGATGGGAACGTGGTCGTCGC | | |
| | | ATGCCTGGTCCAGGGCTTCTTCCCCCATTCGGAGACCTCCCGCTACTC | | |
| | | TGGCGTCGATGAGGGA | | |
| Human- | 31 | CAATCCGCCCTCACTACAACCGGATACGCCGGTCTCGAATGTAATAC | 32 | GGCACGTAGTGAGGTGCAGCTGGTGGAGTCTGGGGGAGGCTTGGTACAG |
| IGHE1 | | GACTCACTATAGGCACGTAGTGAGGTGCAGCTGGTGGAGTCTGGG | | CCTGGGGGGTCCCTGAGACTCTCCTGTGCAGCCTCTGGATTCACCTTCAGTA |
| | | GGAGGCTTGGTACAGCCTGGGGGGTCCCTGAGACTCTCCTGTGCAG | | ACAGTGACATGAACTGGGCCCGCAAGGCTCCAGGAAAGGGGCTGGAGTG |
| | | CCTCTGGATTCACCTTCAGTAACAGTGACATGAACTGGGCCCGCAA | | GGTATCGGGTGTTAGTTGGAATGGCAGTAGGACGCACTATGTGGACTCCG |
| | | GGCTCCAGGAAAGGGGCTGGAGTGGGTATCGGGTGTTAGTTGGAA | | TGAAGCGCCGATTCATCATCTCCAGAGACAATTCCAGGAACTCCCTGTATCT |
| | | TGGCAGTAGGACGCACTATGTGGACTCCGTGAAGCGCCGATTCATC | | GCAAAAGAACAGACGGAGAGCCGAGGACATGGCTGTGTATTACTGTGTG |
| | | ATCTCCAGAGACAATTCCAGGAACTCCCTGTATCTGCAAAAGAACA | | AGAAACCGCAGGAGAGCAGTATTATGATTTTTGGACTGGTTATTATACCC |
| | | GACGGAGAGCCGAGGACATGGCTGTGTATTACTGTGTGAGAAACC | | GGGGGCAGGGTGATGCTTTTGATGTCTGGGGCCAAGGGACAATGGTCACC |
| | | GCAGGAGAGCAGTATTATGATTTTTGGACTGGTTATTATACCCGGG | | GTCTCTTCAGCACGTAGTGCCTCCACACAGAGCCCATCCGTCTTCCCCTTGAC |
| | | GGCAGGGTGATGCTTTTGATGTCTGGGGCCAAGGGACAATGGTCA | | CCGCTGCTGCAAAAACATTCCCTCCAATGCCACCTCCGTGACTCTGGGCTGC |
| | | CCGTCTCTTCAGCACGTAGTGCCTCCACACAGAGCCCATCCGTCTTCC | | CTGGCCACGGGCT |
| | | CCTTGACCCGCTGCTGCAAAAACATTCCCTCCAATGCCACCTCCGTGA | | |
| | | CTCTGGGCTGCCTGGCCACGGGCTATTCGGAGACCTCCCGCTACTCT | | |
| | | GGCGTCGATGAGGGA | | |
| Human- | 33 | CAATCCGCCCTCACTACAACCGGATACGCCGGTCTCGAATGTAATAC | 34 | GGCACGTAGTGAGGTGCAGCTGGTGCAGTCTGGAGCAGAGGTGAAAAAG |
| IGHE2 | | GACTCACTATAGGCACGTAGTGAGGTGCAGCTGGTGCAGTCTGGA | | CCCGGGGAGTCTCTGAAGATCTCCTGTAAGGGTTCTGGATACAGCTTTACC |
| | | GCAGAGGTGAAAAAGCCCGGGGAGTCTCTGAAGATCTCCTGTAAG | | AGCTACTGGATCGGCTGGGTGCACCAGATGCCCGGGAAAGGCCTGGAGTG |
| | | GGTTCTGGATACAGCTTTACCAGCTACTGGATCGGCTGGGTGCACC | | GATGGGGATCATCTATCCTGGTGACTCTGATACCAGATACAGCCCGTCCTTC |
| | | AGATGCCCGGGAAAGGCCTGGAGTGGATGGGGATCATCTATCCTG | | CAAGGCCAGGTCACCATCTCAGCCGACAAGTCCATCAGCACCGCCTACCTGC |
| | | GTGACTCTGATACCAGATACAGCCCGTCCTTCCAAGGCCAGGTCACC | | AGTGGAGCAGCCTGAAGGCCTCGGACACCGCCATGTATTACTGTGCGAGA |
| | | ATCTCAGCCGACAAGTCCATCAGCACCGCCTACCTGCAGTGGAGCA | | CAAGAGGAAGGAAGAGGAGGAGAAGAGAAGAAGAAGGAGAAGGAGA |
| | | GCCTGAAGGCCTCGGACACCGCCATGTATTACTGTGCGAGACAAGA | | AAGAGGAACAAGAAGAGAGAGAGACAAAGACACCAGAAACACAACTGG |
| | | GGAAGGAAGAGGAGGAGAAGAGAAGAAGAAGGAGAAGGAGAA | | TTCGACCCCTGGGGCCAGGGAACCCTGGTCACCGTCTCCTCAGCACGTAGTG |
| | | AGAGGAACAAGAAGAGAGAGAGACAAAGACACCAGAAACACAA | | CCTCCACACAGAGCCCATCCGTCTTCCCCTTGACCCGCTGCTGCAAAAACATT |
| | | CTGGTTCGACCCCTGGGGCCAGGGAACCCTGGTCACCGTCTCCTCAG | | CCCTCCAATGCCACCTCCGTGACTCTGGGCTGCCTGGCCACGGGCT |
| | | CACGTAGTGCCTCCACACAGAGCCCATCCGTCTTCCCCTTGACCCGCT | | |
| | | GCTGCAAAAACATTCCCTCCAATGCCACCTCCGTGACTCTGGGCTGC | | |
| | | CTGGCCACGGGCTATTCGGAGACCTCCCGCTACTCTGGCGTCGATGA | | |
| | | GGGA | | |
| Human- | 35 | CAATCCGCCCTCACTACAACCGGATACGCCGGTCTCGAATGTAATAC | 36 | GGCACGTAGTCAGGTCCAGCTGGTGCAGTCTGGGGCTGAGGTGAAGAAGC |
| IGHG5 | | GACTCACTATAGGCACGTAGTCAGGTCCAGCTGGTGCAGTCTGGGG | | CTGGGGCCTCAGTGAAGGTTTCCTGCAAGGCTTCTGGATACACCTTCACTA |
| | | CTGAGGTGAAGAAGCCTGGGGCCTCAGTGAAGGTTTCCTGCAAGG | | GCTATGCTATGCATTGGGTGCGCCAGGCCCCCGGACAAAGGCTTGAGTGG |
| | | CTTCTGGATACACCTTCACTAGCTATGCTATGCATTGGGTGCGCCAG | | ATGGGATGGATCAACGCTGGCAATGGTAACACAAAATATTCACAGGAGTT |
| | | GCCCCCGGACAAAGGCTTGAGTGGATGGGATGGATCAACGCTGGC | | CCAGGGCAGAGTCACCATTACCAGGGACACATCCGCGAGCACAGCCTACAT |
| | | AATGGTAACACAAAATATTCACAGGAGTTCCAGGGCAGAGTCACC | | GGAGCTGAGCAGCCTGAGATCTGAGGACATGGCTGTGTATTACTGTGCGA |
| | | ATTACCAGGGACACATCCGCGAGCACAGCCTACATGGAGCTGAGCA | | GAGACAGCAGCAGCCGGGAAGAGGAGGAGAAGAGAAGAAGAAGGAGA |
| | | GCCTGAGATCTGAGGACATGGCTGTGTATTACTGTGCGAGAGACA | | AGGAGAAAGAGGAACAAGAAGAGAGAGAGACAAAGACACCAAGAAGT |
| | | GCAGCAGCCGGGAAGAGGAGGAGAAGAGAAGAAGAAGGAGAA | | GATGCTTTTGATATCTGGGGCCAAGGGACAATGGTCACCGTCTCTTCAGCA |
| | | GGAGAAAGAGGAACAAGAAGAGAGAGAGACAAAGACACCAAGA | | CGTAGTGCCTCCACCAAGGGCCCATCGGTCTTCCCCCTGGTGCCCTCCTCCAG |
| | | AGTGATGCTTTTGATATCTGGGGCCAAGGGACAATGGTCACCGTCT | | GAGCGTCTCTGAGGGCACAGCGGCCCTGGGCTGCCTGGTCAAGGACTACTT |
| | | CTTCAGCACGTAGTGCCTCCACCAAGGGCCCATCGGTCTTCCCCCTGG | | CC |
| | | TGCCCTCCTCCAGGAGCGTCTCTGAGGGCACAGCGGCCCTGGGCTGC | | |
| | | CTGGTCAAGGACTACTTCCATTCGGAGACCTCCCGCTACTCTGGCGT | | |
| | | CGATGAGGGA | | |
| Human- | 37 | CAATCCGCCCTCACTACAACCGGATACGCCGGTCTCGAATGTAATAC | 38 | GGCACGTAGTCAGGTCACCTTGAAGGAGTCTGGTCCTGTGCTGGTGAAACC |
| IGHG6 | | GACTCACTATAGGCACGTAGTCAGGTCACCTTGAAGGAGTCTGGTC | | CACACAGACCCTCACACTGACCTGCACCTTCTCTGGGTTCTCACTCAGCACTA |
| | | CTGTGCTGGTGAAACCCACACAGACCCTCACACTGACCTGCACCTTC | | GTGGAATGTGTGTGAGCTGGATCCGTCAGCCCCCAGGGAAGGCCCTGGAG |
| | | TCTGGGTTCTCACTCAGCACTAGTGGAATGTGTGTGAGCTGGATCC | | TGGCTTGCACGCATTGATTGGGATGATGATAAATTCTACAGCACATCTCTG |
| | | GTCAGCCCCCAGGGAAGGCCCTGGAGTGGCTTGCACGCATTGATTG | | AAGACCAGGCTCACCATCTCCAAGGACACCTCCAAAAACCAGGTGGTCCTT |
| | | GGATGATGATAAATTCTACAGCACATCTCTGAAGACCAGGCTCACC | | ACAATGACCAACATGGACCCTGTGGACACAGCCACGTATTACTGTGCACGG |
| | | ATCTCCAAGGACACCTCCAAAAACCAGGTGGTCCTTACAATGACCA | | ATACCAGCAGAGCGCAACACATGACTACAGTAACTACAGGGCGGGACGG |
| | | ACATGGACCCTGTGGACACAGCCACGTATTACTGTGCACGGATACC | | GACTGATGCTTTTGATGTCTGGGGCCAAGGGACAATGGTCACCGTCTCTTC |
| | | AGCAGAGCGCAACACATGACTACAGTAACTACAGGGCGGGACGGG | | AGCACGTAGTGCCTCCACCAAGGGCCCATCGGTCTTCCCCCTGGTGCCCTCCT |
| | | ACTGATGCTTTTGATGTCTGGGGCCAAGGGACAATGGTCACCGTCT | | CCAGGAGCGTCTCTGAGGGCACAGCGGCCCTGGGCTGCCTGGTCAAGGACT |
| | | CTTCAGCACGTAGTGCCTCCACCAAGGGCCCATCGGTCTTCCCCCTGG | | ACTTCC |
| | | TGCCCTCCTCCAGGAGCGTCTCTGAGGGCACAGCGGCCCTGGGCTGC | | |
| | | CTGGTCAAGGACTACTTCCATTCGGAGACCTCCCGCTACTCTGGCGT | | |
| | | CGATGAGGGA | | |
| Human- | 39 | CAATCCGCCCTCACTACAACCGGATACGCCGGTCTCGAATGTAATAC | 40 | GGCACGTAGTGACATCGTGATGACCCAGTCTCCAGACTCCCTGGCTGTGTCT |
| IGK11 | | GACTCACTATAGGCACGTAGTGACATCGTGATGACCCAGTCTCCAG | | CTGGGCGAGAGGGCCACCATCAACTGCAAGTCCAGCCAGAGTGTTTTATAC |
| | | ACTCCCTGGCTGTGTCTCTGGGCGAGAGGGCCACCATCAACTGCAA | | AGCTCCAACAATAAGAACTACTTAGCTTGGTACCAGCAGAAACCAGGACA |
| | | GTCCAGCCAGAGTGTTTTATACAGCTCCAACAATAAGAACTACTTA | | GCCTCCTAAGCTGCTCATTTACTGGGCATCTACCCGGGAATCCGGGGTCCCT |
| | | GCTTGGTACCAGCAGAAACCAGGACAGCCTCCTAAGCTGCTCATTT | | GACCGATTCAGTGGCAGCGGGTCTGGGACAGATTTCACTCTCACCATCAGC |
| | | ACTGGGCATCTACCCGGGAATCCGGGGTCCCTGACCGATTCAGTGG | | AGCCTGCAGGCTGAAGATGTGGCAGTTTATTACTGTCAGCAATATTATAGT |
| | | CAGCGGGTCTGGGACAGATTTCACTCTCACCATCAGCAGCCTGCAG | | ACTCCTCCGCCACCCCGCGCGCTCACGTTCGGCGGAGGGACCAAGGTGGAG |
| | | GCTGAAGATGTGGCAGTTTATTACTGTCAGCAATATTATAGTACTC | | ATCAAACCACGTAGTCGAACTGTGGCTGCACCATCTGTCTTCATCTTCCCGC |
| | | CTCCGCCACCCCGCGCGCTCACGTTCGGCGGAGGGACCAAGGTGGA | | CATCTGATGAGCAGTTGAAATCTGGAACTGCCTCTGTTGTGTG |
| | | GATCAAACCACGTAGTCGAACTGTGGCTGCACCATCTGTCTTCATCT | | |
| | | TCCCGCCATCTGATGAGCAGTTGAAATCTGGAACTGCCTCTGTTGTG | | |
| | | TGATTCGGAGACCTCCCGCTACTCTGGCGTCGATGAGGGA | | |
| Human- | 41 | CAATCCGCCCTCACTACAACCGGATACGCCGGTCTCGAATGTAATAC | 42 | GGCACGTAGTGAAACGACACTCACGCAGTCTCCAGCATTCATGTCAGCGAC |
| IGK49 | | GACTCACTATAGGCACGTAGTGAAACGACACTCACGCAGTCTCCAG | | TCCAGGAGACAAAGTCAACATCTCCTGCAAAGCCAGCCAAGACATTGATG |
| | | CATTCATGTCAGCGACTCCAGGAGACAAAGTCAACATCTCCTGCAA | | ATGATATGAACTGGTACCAACAGAAACCAGGAGAAGCTGCTATTTTCATT |
| | | AGCCAGCCAAGACATTGATGATGATATGAACTGGTACCAACAGAA | | ATTCAAGAAGCTACTACTCTCGTTCCTGGAATCCCACCTCGATTCAGTGGCA |
| | | ACCAGGAGAAGCTGCTATTTTCATTATTCAAGAAGCTACTACTCTCG | | GCGGGTATGGAACAGATTTTACCCTCACAATTAATAACATAGAATCTGAG |
| | | TTCCTGGAATCCCACCTCGATTCAGTGGCAGCGGGTATGGAACAGA | | GATGCTGCATATTACTTCTGTCTACAACATGATAATTTCCCTCTCCAGGAAC |
| | | TTTTACCCTCACAATTAATAACATAGAATCTGAGGATGCTGCATATT | | AACCGCAGGTGCACTTTTGGCCAGGGGACCAAGCTGGAGATCAAACCACG |
| | | ACTTCTGTCTACAACATGATAATTTCCCTCTCCAGGAACAACCGCAG | | TAGTCGAACTGTGGCTGCACCATCTGTCTTCATCTTCCCGCCATCTGATGAG |
| | | GTGCACTTTTGGCCAGGGGACCAAGCTGGAGATCAAACCACGTAGT | | CAGTTGAAATCTGGAACTGCCTCTGTTGTGTG |
| | | CGAACTGTGGCTGCACCATCTGTCTTCATCTTCCCGCCATCTGATGAG | | |
| | | CAGTTGAAATCTGGAACTGCCTCTGTTGTGTGATTCGGAGACCTCCC | | |
| | | GCTACTCTGGCGTCGATGAGGGA | | |
| Human- | \|43 | CAATCCGCCCTCACTACAACCGGATACGCCGGTCTCGAATGTAATAC | 44 | GGCACGTAGTCAGTCTGTGCTGACTCAGCCACCCTCAGCGTCTGGGACCCCC |
| IGL3 | | GACTCACTATAGGCACGTAGTCAGTCTGTGCTGACTCAGCCACCCTC | | GGGCAGAGGGTCACCATCTCTTGTTCTGGAAGCAGCTCCAACATCGGAAGT |
| | | AGCGTCTGGGACCCCCGGGCAGAGGGTCACCATCTCTTGTTCTGGA | | AATTATGTATACTGGTACCAGCAGCTCCCAGGAACGGCCCCCAAACTCCTC |
| | | AGCAGCTCCAACATCGGAAGTAATTATGTATACTGGTACCAGCAGC | | ATCTATAGGAATAATCAGCGGCCCTCAGGGGTCCCTGACCGATTCTCTGGC |
| | | TCCCAGGAACGGCCCCCAAACTCCTCATCTATAGGAATAATCAGCG | | TCCAAGTCTGGCACCTCAGCCTCCCTGGCCATCAGTGGGCTCTGGTCCGAGG |
| | | GCCCTCAGGGGTCCCTGACCGATTCTCTGGCTCCAAGTCTGGCACCTC | | ATGAGGCTGATTATTACTGTGCAGCATGGGATGACAGCCTGAGTGGTCCA |
| | | AGCCTCCCTGGCCATCAGTGGGCTCTGGTCCGAGGATGAGGCTGAT | | AAACCGCACAGCACATGTTTGGCAGCAAGACCCAGCCCACTGTCTTACACG |
| | | TATTACTGTGCAGCATGGGATGACAGCCTGAGTGGTCCAAAACCGC | | TAGTGGTCAGCCCAAGGCTGCCCCCTCGGTCACTCTGTTCCCGCCCTCCTCTG |
| | | ACAGCACATGTTTGGCAGCAAGACCCAGCCCACTGTCTTACACGTA | | AGGAGCTTCAAGCCAACAAGGCCACACTGGTGTGTCTCATA |
| | | GTGGTCAGCCCAAGGCTGCCCCCTCGGTCACTCTGTTCCCGCCCTCCT | | |
| | | CTGAGGAGCTTCAAGCCAACAAGGCCACACTGGTGTGTCTCATAAT | | |
| | | TCGGAGACCTCCCGCTACTCTGGCGTCGATGAGGGA | | |
| Human- | 45 | CAATCCGCCCTCACTACAACCGGATACGCCGGTCTCGAATGTAATAC | 46 | GGCACGTAGTCAGCTTGTGCTGACTCAATCGCCCTCTGCCTCTGCCTCCCTGG |
| IGL5 | | GACTCACTATAGGCACGTAGTCAGCTTGTGCTGACTCAATCGCCCTC | | GAGCCTCGGTCAAGCTCACCTGCACTCTGAGCAGTGGGCACAGCAGCTACG |
| | | TGCCTCTGCCTCCCTGGGAGCCTCGGTCAAGCTCACCTGCACTCTGAG | | CCATCGCATGGCATCAGCAGCAGCCAGAGAAGGGCCCTCGGTACTTGATG |
| | | CAGTGGGCACAGCAGCTACGCCATCGCATGGCATCAGCAGCAGCCA | | AAGCTTAACAGTGATGGCAGCCACAGTAAGGGGGACGGGATCCCTGATCG |
| | | GAGAAGGGCCCTCGGTACTTGATGAAGCTTAACAGTGATGGCAGC | | CTTCTCAGGCTCCAGCTCTGGGGCTGAGCGCTACCTCACCATCTCCAGCCTCC |
| | | CACAGTAAGGGGGACGGGATCCCTGATCGCTTCTCAGGCTCCAGCT | | AGTCTGAGGATGAGGCTGACTATTACTGTCAGACCTGGGGCACTGGCATTC |
| | | CTGGGGCTGAGCGCTACCTCACCATCTCCAGCCTCCAGTCTGAGGAT | | AAAAGGCCAGCACGACGGGGAGAGGCACATGTTTGGCAGCAAGACCCAG |
| | | GAGGCTGACTATTACTGTCAGACCTGGGGCACTGGCATTCAAAAGG | | CCCACTGTCTTACACGTAGTGGTCAGCCCAAGGCTGCCCCCTCGGTCACTCT |
| | | CCAGCACGACGGGGAGAGGCACATGTTTGGCAGCAAGACCCAGCC | | GTTCCCGCCCTCCTCTGAGGAGCTTCAAGCCAACAAGGCCACACTGGTGTGT |
| | | CACTGTCTTACACGTAGTGGTCAGCCCAAGGCTGCCCCCTCGGTCAC | | CTCATA |
| | | TCTGTTCCCGCCCTCCTCTGAGGAGCTTCAAGCCAACAAGGCCACAC | | |
| | | TGGTGTGTCTCATAATTCGGAGACCTCCCGCTACTCTGGCGTCGATG | | |
| | | AGGGA | | |
| Human- | 47 | CAATCCGCCCTCACTACAACCGGATACGCCGGTCTCGAATGTAATAC | 48 | GGCACGTAGTGCCCAGAGAGTGACTCAGCCCGAGAAGCTCCTCTCTGTCTT |
| TRA2 | | GACTCACTATAGGCACGTAGTGCCCAGAGAGTGACTCAGCCCGAGA | | TAAAGGGGCCCCAGTGGAGCTGAAGTGCAACTATTCCTATTCTGGGAGTCC |
| | | AGCTCCTCTCTGTCTTTAAAGGGGCCCCAGTGGAGCTGAAGTGCAA | | TGAACTCTTCTGGTATGTCCAGTACTCCAGACAACGCCTCCAGTTACTCTTG |
| | | CTATTCCTATTCTGGGAGTCCTGAACTCTTCTGGTATGTCCAGTACTC | | AGACACATCTCTAGAGAGAGCATCAAAGGCTTCACTGCTGACCTTAACAA |
| | | CAGACAACGCCTCCAGTTACTCTTGAGACACATCTCTAGAGAGAGC | | AGGCGAGACATCTTTCCACCTGAAGAAACCATTTGCTCAAGAGGAAGACT |
| | | ATCAAAGGCTTCACTGCTGACCTTAACAAAGGCGAGACATCTTTCC | | CAGCCATGTATTACTGTGCTCTAAGTGGCCGAGGGAAGCGACCAGAGTAA |
| | | ACCTGAAGAAACCATTTGCTCAAGAGGAAGACTCAGCCATGTATTA | | TTCAGGGAGCCCAGAAGCTGGTATTTGGCCAAGGAACCAGGCTGACTATC |
| | | CTGTGCTCTAAGTGGCCGAGGGAAGCGACCAGAGTAATTCAGGGA | | AACCCAACACGTAGTATATCCAGAACCCTGACCCTGCCGTGTACCAGCTGA |
| | | GCCCAGAAGCTGGTATTTGGCCAAGGAACCAGGCTGACTATCAACC | | GAGACTCTAAATCCAGTGACAAGTCTGTCTGCCTATTCACCGATTTTGATT |
| | | CAACACGTAGTATATCCAGAACCCTGACCCTGCCGTGTACCAGCTGA | | |
| | | GAGACTCTAAATCCAGTGACAAGTCTGTCTGCCTATTCACCGATTTT | | |
| | | GATTATTCGGAGACCTCCCGCTACTCTGGCGTCGATGAGGGA | | |
| Human- | 49 | CAATCCGCCCTCACTACAACCGGATACGCCGGTCTCGAATGTAATAC | 50 | GGCACGTAGTAAAAACCAAGTGGAGCAGAGTCCTCAGTCCCTGATCATCCT |
| TRA3 | | GACTCACTATAGGCACGTAGTAAAAACCAAGTGGAGCAGAGTCCT | | GGAGGGAAAGAACTGCACTCTTCAATGCAATTATACAGTGAGCCCCTTCA |
| | | CAGTCCCTGATCATCCTGGAGGGAAAGAACTGCACTCTTCAATGCA | | GCAACTTAAGGTGGTATAAGCAAGATACTGGGAGAGGTCCTGTTTCCCTG |
| | | ATTATACAGTGAGCCCCTTCAGCAACTTAAGGTGGTATAAGCAAGA | | ACAATCATGACTTTCAGTGAGAACACAAAGTCGAACGGAAGATATACAGC |
| | | TACTGGGAGAGGTCCTGTTTCCCTGACAATCATGACTTTCAGTGAG | | AACTCTGGATGCAGACACAAAGCAAAGCTCTCTGCACATCACAGCCTCCCA |
| | | AACACAAAGTCGAACGGAAGATATACAGCAACTCTGGATGCAGAC | | GCTCAGCGATTCAGCCTCCTACATCTGTGTGGTGAGCGAGCAGCGACCGGG |
| | | ACAAAGCAAAGCTCTCTGCACATCACAGCCTCCCAGCTCAGCGATTC | | ATGACAACTGACAGCTGGGGGAAATTGCAGTTTGGAGCAGGGACCCAGG |
| | | AGCCTCCTACATCTGTGTGGTGAGCGAGCAGCGACCGGGATGACAA | | TTGTGGTCACCCCAGCACGTAGTATATCCAGAACCCTGACCCTGCCGTGTAC |
| | | CTGACAGCTGGGGGAAATTGCAGTTTGGAGCAGGGACCCAGGTTG | | CAGCTGAGAGACTCTAAATCCAGTGACAAGTCTGTCTGCCTATTCACCGAT |
| | | TGGTCACCCCAGCACGTAGTATATCCAGAACCCTGACCCTGCCGTGT | | TTTGATT |
| | | ACCAGCTGAGAGACTCTAAATCCAGTGACAAGTCTGTCTGCCTATTC | | |
| | | ACCGATTTTGATTATTCGGAGACCTCCCGCTACTCTGGCGTCGATGA | | |
| | | GGGA | | |

-

| | | | | |
|---|---|---|---|---|
| Human | 51 | CAATCCGCCCTCACTACAACCGGATACGCCGGTCTCGAATGTAATAC | 52 | GGCACGTAGTAATGCTGGTGTCACTCAGACCCCAAAATTCCACGTCCTGAA |
| TRB6 | | GACTCACTATAGGCACGTAGTAATGCTGGTGTCACTCAGACCCCAA | | GACAGGACAGAGCATGACTCTGCTGTGTGCCCAGGATATGAACCATGAAT |
| | | AATTCCACGTCCTGAAGACAGGACAGAGCATGACTCTGCTGTGTGC | | ACATGTATCGGTATCGACAAGACCCAGGCAAGGGGCTGAGGCTGATTTAC |
| | | CCAGGATATGAACCATGAATACATGTATCGGTATCGACAAGACCCA | | TACTCAGTTGCTGCTGCTCTCACTGACAAAGGAGAAGTTCCCAATGGCTAC |
| | | GGCAAGGGGCTGAGGCTGATTTACTACTCAGTTGCTGCTGCTCTCAC | | AATGTCTCCAGATCAAACACAGAGGATTTCCCCCTCAAGCTGGAGTCAGCT |
| | | TGACAAAGGAGAAGTTCCCAATGGCTACAATGTCTCCAGATCAAAC | | GCTCCCTCTCAGACTTCTGTTTACTTCTGTGCCAGCAGTTACTCAGCGGGAG |
| | | ACAGAGGATTTCCCCCTCAAGCTGGAGTCAGCTGCTCCCTCTCAGAC | | GCCGGGGGACAGGGGGCAACCAGCCAAAAACATTCAGTACTTCGGCGCCG |
| | | TTCTGTTTACTTCTGTGCCAGCAGTTACTCAGCGGGAGGCCGGGGG | | GGACCCGGCTCTCAGTGCTGGCACGTAGTGAGGACCTGAACAAGGTGTTCC |
| | | ACAGGGGGCAACCAGCCAAAAACATTCAGTACTTCGGCGCCGGGA | | CACCCGAGGTCGCTGTGTTTGAGCCATCAGAAGCAGAGATCTCCCACACCC |
| | | CCCGGCTCTCAGTGCTGGCACGTAGTGAGGACCTGAACAAGGTGTT | | AAAAGGCCACACTGGTG |
| | | CCCACCCGAGGTCGCTGTGTTTGAGCCATCAGAAGCAGAGATCTCCC | | |
| | | ACACCCAAAAGGCCACACTGGTGATTCGGAGACCTCCCGCTACTCTG | | |
| | | GCGTCGATGAGGGA | | |
| Human- | 53 | CAATCCGCCCTCACTACAACCGGATACGCCGGTCTCGAATGTAATAC | 54 | GGCACGTAGTGACACAGCTGTTTCCCAGACTCCAAAATACCTGGTCACACA |
| TRB13 | | GACTCACTATAGGCACGTAGTGACACAGCTGTTTCCCAGACTCCAA | | GATGGGAAACGACAAGTCCATTAAATGTGAACAAAATCTGGGCCATGAT |
| | | AATACCTGGTCACACAGATGGGAAACGACAAGTCCATTAAATGTG | | ACTATGTATTGGTATAAACAGGACTCTAAGAAATTTCTGAAGATAATGTT |
| | | AACAAAATCTGGGCCATGATACTATGTATTGGTATAAACAGGACTC | | TAGCTACAATAATAAGGAGCTCATTATAAATGAAACAGTTCCAAATCGCT |
| | | TAAGAAATTTCTGAAGATAATGTTTAGCTACAATAATAAGGAGCT | | TCTCACCTAAATCTCCAGACAAAGCTCACTTAAATCTTCACATCAATTCCCT |
| | | CATTATAAATGAAACAGTTCCAAATCGCTTCTCACCTAAATCTCCAG | | GGAGCTTGGTGACTCTGCTGTGTATTTCTGTGCCAGCAGCCAAGAGAAGG |
| | | ACAAAGCTCACTTAAATCTTCACATCAATTCCCTGGAGCTTGGTGAC | | ACAGGGGACAGGGGGCGAAACGACCTCCTACGAGCAGTACTTCGGGCCGG |
| | | TCTGCTGTGTATTTCTGTGCCAGCAGCCAAGAGAAGGACAGGGGAC | | GCACCAGGCTCACGGTCACAGCACGTAGTGAGGACCTGAACAAGGTGTTC |
| | | AGGGGGCGAAACGACCTCCTACGAGCAGTACTTCGGGCCGGGCACC | | CCACCCGAGGTCGCTGTGTTTGAGCCATCAGAAGCAGAGATCTCCCACACC |
| | | AGGCTCACGGTCACAGCACGTAGTGAGGACCTGAACAAGGTGTTCC | | CAAAAGGCCACACTGGTG |
| | | CACCCGAGGTCGCTGTGTTTGAGCCATCAGAAGCAGAGATCTCCCA | | |
| | | CACCCAAAAGGCCACACTGGTGATTCGGAGACCTCCCGCTACTCTGG | | |
| | | CGTCGATGAGGGA | | |
| Human- | 55 | CAATCCGCCCTCACTACAACCGGATACGCCGGTCTCGAATGTAATAC | 56 | GGCACGTAGTTGTGACAAAGTAACCCAGAGTTCCCCGGACCAGACGGTGG |
| TRD1 | | GACTCACTATAGGCACGTAGTTGTGACAAAGTAACCCAGAGTTCCC | | CGAGTGGCAGTGAGGTGGTACTGCTCTGCACTTACGACACTGTATATTCAA |
| | | CGGACCAGACGGTGGCGAGTGGCAGTGAGGTGGTACTGCTCTGCA | | ATCCAGATTTATTCTGGTACTGGATAAGGCCAGATTATTCCTTTCAGTTTGT |
| | | CTTACGACACTGTATATTCAAATCCAGATTTATTCTGGTACTGGATA | | CTTTTATGGGGATAACAGCAGATCAGAAGGTGCAGATTTTACTCAAGGAC |
| | | AGGCCAGATTATTCCTTTCAGTTTGTCTTTTATGGGGATAACAGCAG | | GGTTTTCTGTGAAACACATTCTGACCCAGAAAGCCTTTCACTTGGTGATCTC |
| | | ATCAGAAGGTGCAGATTTTACTCAAGGACGGTTTTCTGTGAAACAC | | TCCAGTAAGGACTGAAGACAGTGCCACTTACTACTGTGCCTTTAGACAAGG |
| | | ATTCTGACCCAGAAAGCCTTTCACTTGGTGATCTCTCCAGTAAGGAC | | CCCAAGGACGACTGGGGGATACGAAAACACGCCAGACCCCTGATCTTTGG |
| | | TGAAGACAGTGCCACTTACTACTGTGCCTTTAGACAAGGCCCAAGG | | CAAAGGAACCTATCTGGAGGTACAACAACCACGTAGTGAAGTCAGCCTCA |
| | | ACGACTGGGGGATACGAAAACACGCCAGACCCCTGATCTTTGGCAA | | TACCAAACCATCCGTTTTTGTCATGAAAAATGGAACAAATGTCGCTTGTCT |
| | | AGGAACCTATCTGGAGGTACAACAACCACGTAGTGAAGTCAGCCTC | | GGTGAAGGAATTCTACCCCAAGGA |
| | | ATACCAAACCATCCGTTTTTGTCATGAAAAATGGAACAAATGTCGC | | |
| | | TTGTCTGGTGAAGGAATTCTACCCCAAGGAATTCGGAGACCTCCCG | | |
| | | CTACTCTGGCGTCGATGAGGGA | | |
| Human- | 57 | CAATCCGCCCTCACTACAACCGGATACGCCGGTCTCGAATGTAATAC | 58 | GGCACGTAGTATTGAGTTGGTGCCTGAACACCAAACAGTGCCTGTGTCAAT |
| TRD2 | | GACTCACTATAGGCACGTAGTATTGAGTTGGTGCCTGAACACCAAA | | AGGGATCCCTGCCACCCTCAGGTGCTCCATGAAAGGAGAAGCGATCGGTA |
| | | CAGTGCCTGTGTCAATAGGGATCCCTGCCACCCTCAGGTGCTCCATG | | ACTACTATATCAACTGGTACAGGAAGACCCAAGGTAACACAATCACTTTCA |
| | | AAAGGAGAAGCGATCGGTAACTACTATATCAACTGGTACAGGAAG | | TATACCGAGAAAAGGACATCTATGGCCCTGGTTTCAAAGACAATTTCCAA |
| | | ACCCAAGGTAACACAATCACTTTCATATACCGAGAAAAGGACATCT | | GGTGACATTGATATTGCAAAGAACCTGGCTGTACTTAAGATACTTGCACCA |
| | | ATGGCCCTGGTTTCAAAGACAATTTCCAAGGTGACATTGATATTGC | | TCAGAGAGAGATGAAGGGTCTTACTACTGTGCCTGTGACACCCAACGAGA |
| | | AAAGAACCTGGCTGTACTTAAGATACTTGCACCATCAGAGAGAGA | | CGCCGCCTTCCTACCGGACAGAACCGCTTTGACAGCACAACTCTTCTTTGGA |
| | | TGAAGGGTCTTACTACTGTGCCTGTGACACCCAACGAGACGCCGCCT | | AAGGGAACACAACTCATCGTGGAACCAGCACGTAGTGAAGTCAGCCTCAT |
| | | TCCTACCGGACAGAACCGCTTTGACAGCACAACTCTTCTTTGGAAAG | | ACCAAACCATCCGTTTTTGTCATGAAAAATGGAACAAATGTCGCTTGTCTG |
| | | GGAACACAACTCATCGTGGAACCAGCACGTAGTGAAGTCAGCCTCA | | GTGAAGGAATTCTACCCCAAGGA |
| | | TACCAAACCATCCGTTTTTGTCATGAAAAATGGAACAAATGTCGCT | | |
| | | TGTCTGGTGAAGGAATTCTACCCCAAGGAATTCGGAGACCTCCCGC | | |
| | | TACTCTGGCGTCGATGAGGGA | | |
| Human- | 59 | CAATCCGCCCTCACTACAACCGGATACGCCGGTCTCGAATGTAATAC | 60 | GGCACGTAGTTCTTCCAACTTGGAAGGGAGAACGAAGTCAGTCACCAGGC |
| TRG2 | | GACTCACTATAGGCACGTAGTTCTTCCAACTTGGAAGGGAGAACGA | | AGACTGGGTCATCTGCTGAAATCACTTGCGATCTTACTGTAACAAATACCT |
| | | AGTCAGTCACCAGGCAGACTGGGTCATCTGCTGAAATCACTTGCGA | | TCTACATCCACTGGTACCTACACCAGGAGGGGAAGGCCCCACAGCGTCTTC |
| | | TCTTACTGTAACAAATACCTTCTACATCCACTGGTACCTACACCAGG | | TGTACTATGACGTCTCCACTGCAAGGGATGTGTTGGAATCAGGACTCAGTC |
| | | AGGGGAAGGCCCCACAGCGTCTTCTGTACTATGACGTCTCCACTGCA | | CAGGAAAGTATTATACTCATACACCCAGGAGGTGGAGCTGGATATTGAGA |
| | | AGGGATGTGTTGGAATCAGGACTCAGTCCAGGAAAGTATTATACT | | CTGCAAAATCTAATTGAAAATGATTCTGGGGTCTATTACTGTGCCACCTGG |
| | | CATACACCCAGGAGGTGGAGCTGGATATTGAGACTGCAAAATCTA | | GACAGCGACGCAGAACCCGGACCAGCATACCACTGGTTGGTTCAAGATAT |
| | | ATTGAAAATGATTCTGGGGTCTATTACTGTGCCACCTGGGACAGCG | | TTGCTGAAGGGACTAAGCTCATAGTAACTTCACCTGCACGTAGTGATAAAC |
| | | ACGCAGAACCCGGACCAGCATACCACTGGTTGGTTCAAGATATTTG | | AACTTGATGCAGATGTTTCCCCCAAGCCCACTATTTTTCTTCCTTCGATTGCT |
| | | CTGAAGGGACTAAGCTCATAGTAACTTCACCTGCACGTAGTGATAA | | GAAACAAAACTCCAGAAGGCTGGAACATAC |
| | | ACAACTTGATGCAGATGTTTCCCCCAAGCCCACTATTTTTCTTCCTTC | | |
| | | GATTGCTGAAACAAAACTCCAGAAGGCTGGAACATACATTCGGAG | | |
| | | ACCTCCCGCTACTCTGGCGTCGATGAGGGA | | |
| Human- | 61 | CAATCCGCCCTCACTACAACCGGATACGCCGGTCTCGAATGTAATAC | 62 | GGCACGTAGTTCTTCCAACTTGGAAGGGAGAACGAAGTCAGTCACCAGGC |
| TRG3 | | GACTCACTATAGGCACGTAGTTCTTCCAACTTGGAAGGGAGAACGA | | TGACTGGGTCATCTGCTGAAATCACCTGTGATCTTCCTGGAGCAAGTACCTT |
| | | AGTCAGTCACCAGGCTGACTGGGTCATCTGCTGAAATCACCTGTGA | | ATACATCCACTGGTACCTGCACCAGGAGGGGAAGGCCCCACAGTGTCTTCT |
| | | TCTTCCTGGAGCAAGTACCTTATACATCCACTGGTACCTGCACCAGG | | GTACTATGAACCCTACTACTCCAGGGTTGTGCTGGAATCAGGAATCACTCC |
| | | AGGGGAAGGCCCCACAGTGTCTTCTGTACTATGAACCCTACTACTCC | | AGGAAAGTATGACACTGGAAGCACAAGGAGCAATTGGAATTTGAGACTG |
| | | AGGGTTGTGCTGGAATCAGGAATCACTCCAGGAAAGTATGACACT | | CAAAATCTAATTAAAAATGATTCTGGGTTCTATTACTGTGCCACCTGGGAC |
| | | GGAAGCACAAGGAGCAATTGGAATTTGAGACTGCAAAATCTAATT | | AGGAGACGCACCGAAGAATTATTATAAGAAACTCTTTGGCAGTGGAACA |
| | | AAAAATGATTCTGGGTTCTATTACTGTGCCACCTGGGACAGGAGAC | | ACACTGGTTGTCACAGCACGTAGTGATAAACAACTTGATGCAGATGTTTCC |
| | | GCACCGAAGAATTATTATAAGAAACTCTTTGGCAGTGGAACAACA | | CCCAAGCCCACTATTTTTCTTCCTTCGATTGCTGAAACAAAACTCCAGAAGG |
| | | CTGGTTGTCACAGCACGTAGTGATAAACAACTTGATGCAGATGTTT | | CTGGAACATAC |
| | | CCCCCAAGCCCACTATTTTTCTTCCTTCGATTGCTGAAACAAAACTCC | | |
| | | AGAAGGCTGGAACATACATTCGGAGACCTCCCGCTACTCTGGCGTC | | |
| | | GATGAGGGA | | |

in silico V(D)J combination

| | | | | |
|---|---|---|---|---|
| Human- | 63 | CAATCCGCCCTCACTACAACCGGATACGCCGGTCTCGAATGTAATAC | 64 | GGCACGTAGTAGAGAGCGTGTCATTATGTGCCGGCACAGGAGTCTAGAGT |
| IGHM9_ | | GACTCACTATAGGCACGTAGTAGAGAGCGTGTCATTATGTGCCGGC | | CCGACGAGTCGAGGTACATCCGACACGAGCACCTAAGCAGGCGCCATTAG |
| rev | | ACAGGAGTCTAGAGTCCGACGAGTCGAGGTACATCCGACACGAGC | | CACTGAGACGGGACCTTGAAGACACGCATCAAACGACATGGTTTCTATCCC |
| | | ACCTAAGCAGGCGCCATTAGCACTGAGACGGGACCTTGAAGACAC | | TACTAGGGAGGGTAGGTGAGTTCGGGAACAGGTCCCCGGACAGCGTGGG |
| | | GCATCAAACGACATGGTTTCTATCCCTACTAGGGAGGGTAGGTGAG | | TCGACTATCGTATCGACGACTTCCACGGAGGTCTTCGGAACGTCCTCTGGA |
| | | TTCGGGAACAGGTCCCCGGACAGCGTGGGTCGACTATCGTATCGAC | | AGTGGCTCCTGGGTCCGAAGAAGTGGAGTCGGGGTCTGACGTGGTCGACC |
| | | GACTTCCACGGAGGTCTTCGGAACGTCCTCTGGAAGTGGCTCCTGG | | TGGACACAACCACACGGCATCAATGACATCAGTGGGAGGGACTCCTCTGCC |
| | | GTCCGAAGAAGTGGAGTCGGGGTCTGACGTGGTCGACCTGGACAC | | ACTGGCACCAGGGAACGGGGGTCTGCAGGTATGGCATCATCATCATCATT |
| | | AACCACACGGCATCAATGACATCAGTGGGAGGGACTCCTCTGCCAC | | ACACGTAGTGGGAGTGCATCCGCCCCAACCCTTTTCCCCCTCGTCTCCTGTGA |
| | | TGGCACCAGGGAACGGGGGTCTGCAGGTATGGCATCATCATCATCA | | GAATTCCCCGTCGGATACGAGCAGCGTGGCCGTTGGCTGCCTCGCACAGGA |
| | | TTACACGTAGTGGGAGTGCATCCGCCCCAACCCTTTTCCCCCTCGTCT | | CTTCC |
| | | CCTGTGAGAATTCCCCGTCGGATACGAGCAGCGTGGCCGTTGGCTG | | |
| | | CCTCGCACAGGACTTCCATTCGGAGACCTCCCGCTACTCTGGCGTCG | | |
| | | ATGAGGGA | | |
| Human- | 65 | CAATCCGCCCTCACTACAACCGGATACGCCGGTCTCGAATGTAATAC | 66 | GGCACGTAGTCACAGGCGCCGCCAGTGTCTCGAGTCGAAGTCCCTCTTGAC |
| IGHM12_ | | GACTCACTATAGGCACGTAGTCACAGGCGCCGCCAGTGTCTCGAGT | | CAAGAACCTGCACAGATGCCTATACCACTGAGCTGAGAACTCCCTGCCCAA |
| rev | | CGAAGTCCCTCTTGACCAAGAACCTGCACAGATGCCTATACCACTGA | | CATCATCCACGAGGGTGATATTATCTATGAGGGTTAGGTGAGGTCGGGGA |
| | | GCTGAGAACTCCCTGCCCAACATCATCCACGAGGGTGATATTATCTA | | AGGGACCCCCGACCGCCTAGGTCGGGGTCATCATTGATGATGACGACTACC |
| | | TGAGGGTTAGGTGAGGTCGGGGAAGGGACCCCCGACCGCCTAGGT | | TCGCGGCGAACCCAGACGGAACCACAGAAACAGAGAGAGAGAAGAACA |
| | | CGGGGTCATCATTGATGATGACGACTACCTCGCGGCGAACCCAGAC | | AGGAGAAAGAGGAAGAGGAAGAAGAAGAGAAGAGGAGGAGAAGGCA |
| | | GGAACCACAGAAACAGAGAGAGAGAAGAACAAGGAGAAAGAGG | | GAACCGAAAGGGACTCCTCTGCCACTGGTCCCAAGGAACCGGGGTCATCA |
| | | AAGAGGAAGAAGAAGAGAAGAGGAGGAGAAGGCAGAACCGAA | | GTTTCATCACACGTAGTGGGAGTGCATCCGCCCCAACCCTTTTCCCCCTCGTC |
| | | AGGGACTCCTCTGCCACTGGTCCCAAGGAACCGGGGTCATCAGTTTC | | TCCTGTGAGAATTCCCCGTCGGATACGAGCAGCGTGGCCGTTGGCTGCCTC |
| | | ATCACACGTAGTGGGAGTGCATCCGCCCCAACCCTTTTCCCCCTCGTC | | GCACAGGACTTCC |
| | | TCCTGTGAGAATTCCCCGTCGGATACGAGCAGCGTGGCCGTTGGCT | | |
| | | GCCTCGCACAGGACTTCCATTCGGAGACCTCCCGCTACTCTGGCGTC | | |
| | | GATGAGGGA | | |
| Human- | 67 | CAATCCGCCCTCACTACAACCGGATACGCCGGTCTCGAATGTAATAC | 68 | GGCACGTAGTGGAGAGCGTGTCATTATGTGTCGGCACAGGCGCCGCCAGT |
| IGHM14_ | | GACTCACTATAGGCACGTAGTGGAGAGCGTGTCATTATGTGTCGGC | | GTCTCGAGTCGAAGTCCCTCTTGACCAAGAACCTGCACAGATGACTATACC |
| rev | | ACAGGCGCCGCCAGTGTCTCGAGTCGAAGTCCCTCTTGACCAAGAA | | ACCGAGCTGAGAACTCCCTGCCCAACAACAACCACGAAGGTGATACTAACT |
| | | CCTGCACAGATGACTATACCACCGAGCTGAGAACTCCCTGCCCAACA | | AAAGGGGTTAGGTGAGGTCGGGGAAGGGATCCCCGACCGCCTAGGTCGT |
| | | ACAACCACGAAGGTGATACTAACTAAAGGGGTTAGGTGAGGTCGG | | GGTCATCATTGGTGACTTCCTGGGTGGTATCTGTCGCGTCCACTCCCTGTCCC |
| | | GGAAGGGATCCCCGACCGCCTAGGTCGTGGTCATCATTGGTGACTT | | |
| | | CCTGGGTGGTATCTGTCGCGTCCACTCCCTGTCCCAGAGGCTTCCGA | | |
| | | AGTTGTCAGGACGCGGGGTGACGACATCGACGTGGACGCCCGAGG | | AGAGGCTTCCGAAGTTGTCAGGACGCGGGGTGACGACATCGACGTGGACG |
| | | AAACAGCATTAGCATCTGTGATATAGGTGAGCGGAAGACCACAGA | | CCCGAGGAAACAGCATTAGCATCTGTGATATAGGTGAGCGGAAGACCACA |
| | | CTCCTCTGCCACTGGTCCCAGGGAACCGGGGTCATCAGTTTCATCGC | | GACTCCTCTGCCACTGGTCCCAGGGAACCGGGGTCATCAGTTTCATCGCACG |
| | | ACGTAGTGGGAGTGCATCCGCCCCAACCCTTTTCCCCCTCGTCTCCTG | | TAGTGGGAGTGCATCCGCCCCAACCCTTTTCCCCCTCGTCTCCTGTGAGAATT |
| | | TGAGAATTCCCCGTCGGATACGAGCAGCGTGGCCGTTGGCTGCCTC | | CCCCGTCGGATACGAGCAGCGTGGCCGTTGGCTGCCTCGCACAGGACTTCC |
| | | GCACAGGACTTCCATTCGGAGACCTCCCGCTACTCTGGCGTCGATGA | | |
| | | GGGA | | |
| Human- | 69 | CAATCCGCCCTCACTACAACCGGATACGCCGGTCTCGAATGTAATAC | 70 | GGCACGTAGTGGAGAGCGTGTCATTATGTGTCGGCACAGGCGCCGCCAGT |
| IGHD6_ | | GACTCACTATAGGCACGTAGTGGAGAGCGTGTCATTATGTGTCGGC | | GTCTCGAGTCGAAGTCCCTCTTGACCAAGAACCTGCACAGATGACTATACC |
| rev | | ACAGGCGCCGCCAGTGTCTCGAGTCGAAGTCCCTCTTGACCAAGAA | | ACTGAGCTGAGAACTCCCTGCCCAACATCAACCACGAAGGTGATACTAACT |
| | | CCTGCACAGATGACTATACCACTGAGCTGAGAACTCCCTGCCCAACA | | AAAGGGGTTAGGTGAGGTCGGGGAAGGGACCCCCGACCGCCTAGGTCGA |
| | | TCAACCACGAAGGTGATACTAACTAAAGGGGTTAGGTGAGGTCGG | | GGTCATCATTGGTGACTTCCTGGGTGGTATCTGTCGCGTCCACTCCCTGTCCC |
| | | GGAAGGGACCCCCGACCGCCTAGGTCGAGGTCATCATTGGTGACTT | | AGAGGCTTCCGAAGTTGTCAGGACGCGGGGTGACAACATCGACGTGGACC |
| | | CCTGGGTGGTATCTGTCGCGTCCACTCCCTGTCCCAGAGGCTTCCGA | | GCCCGCAGCGCAGACCACAGAAACAGAGAGAGAGAAGAACAAGGAGAA |
| | | AGTIGTCAGGACGCGGGGTGACAACATCGACGTGGACCGCCCGCA | | AGAGGAAGAGGAAGAAGAAGAGAAGAGGAGGAGAAGGGCAGCAAGA |
| | | GCGCAGACCACAGAAACAGAGAGAGAGAAGAACAAGGAGAAAG | | CTCCTCTGCCACTGGTCCCAGGGAACCGGGGTCATCAGTTTCATCGCACGTA |
| | | AGGAAGAGGAAGAAGAAGAGAAGAGGAGGAGAAGGGCAGCAA | | GTGCACCCACCAAGGCTCCGGATGTGTTCCCCATCATATCAGGGTGCAGAC |
| | | GACTCCTCTGCCACTGGTCCCAGGGAACCGGGGTCATCAGTTTCATC | | ACCCAAAGGATAACAGCCCTGTGGTCCTGGCATGCTTGATAACTGGGTACC |
| | | GCACGTAGTGCACCCACCAAGGCTCCGGATGTGTTCCCCATCATATC | | |
| | | AGGGTGCAGACACCCAAAGGATAACAGCCCTGTGGTCCTGGCATGC | | |
| | | TTGATAACTGGGTACCATTCGGAGACCTCCCGCTACTCTGGCGTCGA | | |
| | | TGAGGGA | | |
| Human- | 71 | CAATCCGCCCTCACTACAACCGGATACGCCGGTCTCGAATGTAATAC | 72 | GGCACGTAGTAGAGAGCGTGTCGTTATGTGCCGGCACAGGCGCCGCCAGT |
| IGHD8_ | | GACTCACTATAGGCACGTAGTAGAGAGCGTGTCGTTATGTGCCGGC | | GTCTCGAGTCCAAGTCCCTCTTGACCAAGAACCTGCACAGATGACTATACC |
| rev | | ACAGGCGCCGCCAGTGTCTCGAGTCCAAGTCCCTCTTGACCAAGAAC | | ACCGAGCTGAGAACTCCCTCCCCAACAACAACCACGAGGGTGATATTATCT |
| | | CTGCACAGATGACTATACCACCGAGCTGAGAACTCCCTCCCCAACAA | | ATATGGGTTAGGTGAGGTCGGGGAAGGGACCCCCGACGGCCTAGGTCGA |
| | | CAACCACGAGGGTGATATTATCTATATGGGTTAGGTGAGGTCGGG | | GGTCATCATTGGTGACTGCCTGGGTGGTATCTGTCGCGTCCACTCCCTGTCC |
| | | GAAGGGACCCCCGACGGCCTAGGTCGAGGTCATCATTGGTGACTGC | | CAGAGGCTTCCGAAGTTGTCAGGACGCGGGGTGACGACATCGACGTGGAC |
| | | CTGGGTGGTATCTGTCGCGTCCACTCCCTGTCCCAGAGGCTTCCGAA | | GGACGGCCGGCAGCAAGGTCAATATGGACGGGACTCCTCTGCCACTGGTCC |
| | | GTTGTCAGGACGCGGGGTGACGACATCGACGTGGACGGACGGCCG | | CAAGGGACCGGGGTCATCAGTTTCATCACACGTAGTGCACCCACCAAGGCT |
| | | GCAGCAAGGTCAATATGGACGGGACTCCTCTGCCACTGGTCCCAAG | | CCGGATGTGTTCCCCATCATATCAGGGTGCAGACACCCAAAGGATAACAGC |
| | | GGACCGGGGTCATCAGTTTCATCACACGTAGTGCACCCACCAAGGC | | CCTGTGGTCCTGGCATGCTTGATAACTGGGTACC |
| | | TCCGGATGTGTTCCCCATCATATCAGGGTGCAGACACCCAAAGGAT | | |
| | | AACAGCCCTGTGGTCCTGGCATGCTTGATAACTGGGTACCATTCGG | | |
| | | AGACCTCCCGCTACTCTGGCGTCGATGAGGGA | | |
| Human- | 73 | CAATCCGCCCTCACTACAACCGGATACGCCGGTCTCGAATGTAATAC | 74 | GGCACGTAGTTTTAGGACAGGTGAGTTCGGGAACAGGTCCCTGGGTAGCG |
| IGHD10_ | | GACTCACTATAGGCACGTAGTTTTAGGACAGGTGAGTTCGGGAAC | | TGGGTCACGTTCATCGTCATCCACTTCCACATAGGTCTTCGGAACGTCCTCT |
| rev | | AGGTCCCTGGGTAGCGTGGGTCACGTTCATCGTCATCCACTTCCACA | | GGAAGTGACTCCGGGGTCCGAAGAAGTGGAGTCGGGGTCTGACGTGGTC |
| | | TAGGTCTTCGGAACGTCCTCTGGAAGTGACTCCGGGGTCCGAAGAA | | GACGTGGTCACGGGCGACACGCACACAATGCCCACCACCGACCCCGACACC |
| | | GTGGAGTCGGGGTCTGACGTGGTCGACGTGGTCACGGGCGACACG | | AACGGAACCGCTCCGACGGGAGACGAACCCCAACACGTCACCCGTGACTCC |
| | | CACACAATGCCCACCACCGACCCCGACACCAACGGAACCGCTCCGAC | | TCCGGACACGGAAACCTCTGAGCCACGCCGGAGACTCCTCTGCCACTGGCA |
| | | GGGAGACGAACCCCAACACGTCACCCGTGACTCCTCCGGACACGGA | | CCAGGGAACCGGGGTCTGCAGGTATGGCATCATCATCATCATTACACGTAG |
| | | AACCTCTGAGCCACGCCGGAGACTCCTCTGCCACTGGCACCAGGGA | | TGCACCCACCAAGGCTCCGGATGTGTTCCCCATCATATCAGGGTGCAGACA |
| | | ACCGGGGTCTGCAGGTATGGCATCATCATCATCATTACACGTAGTG | | CCCAAAGGATAACAGCCCTGTGGTCCTGGCATGCTTGATAACTGGGTACC |
| | | CACCCACCAAGGCTCCGGATGTGTTCCCCATCATATCAGGGTGCAGA | | |
| | | CACCCAAAGGATAACAGCCCTGTGGTCCTGGCATGCTTGATAACTG | | |
| | | GGTACCATTCGGAGACCTCCCGCTACTCTGGCGTCGATGAGGGA | | |
| Human- | 75 | CAATCCGCCCTCACTACAACCGGATACGCCGGTCTCGAATGTAATAC | 76 | GGCACGTAGTAGAGCGTGTCATTATGTACCGCCACAGGCTCCGGAAGTCCG |
| IGHA11_ | | GACTCACTATAGGCACGTAGTAGAGCGTGTCATTATGTACCGCCAC | | ACGAGGTGACGTCCATCCGCCACGACTACCCGAACAGCCGACTCTACCACT |
| rev | | AGGCTCCGGAAGTCCGACGAGGTGACGTCCATCCGCCACGACTACC | | GGACCGGAACCTTCCTGCCCGACATAGACCATAGTCTCAGTGGTCCTATCTA |
| | | CGAACAGCCGACTCTACCACTGGACCGGAACCTTCCTGCCCGACATA | | CTAGGGGTAGGTGAGGTCCGGAAAGGGCCCGTAGACCGCGTGGGTCGGCT |
| | | GACCATAGTCTCAGTGGTCCTATCTACTAGGGGTAGGTGAGGTCCG | | AGGTCATCGACCATTTCGACATAGGTCTTGGGAATGTCCTCTAGAAGTCTC |
| | | GAAAGGGCCCGTAGACCGCGTGGGTCGGCTAGGTCATCGACCATTT | | TGAGGGGGCCGAAAAAGTGGAGACGAGGTCTGACGTGGTCGACGTGGAG |
| | | CGACATAGGTCTTGGGAATGTCCTCTAGAAGTCTCTGAGGGGGCCG | | |
| | | AAAAAGTGGAGACGAGGTCTGACGTGGTCGACGTGGAGGGGGCC | | |
| | | AGGCCACCACAGAAACAGAGAGAGAGAAGAACAAGGAGAAAGA | | GGGGCCAGGCCACCACAGAAACAGAGAGAGAGAAGAACAAGGAGAAAG |
| | | GGAAGAGGAAGAAGAAGAGAAGAGGAGGAGAAGGGCGAAAGC | | AGGAAGAGGAAGAAGAAGAGAAGAGGAGGAGAAGGGCGAAAGCAGA |
| | | AGACTCCTCTGCCACTGGTCCCAAGGGACCGGGGTCATCAGTTTCAT | | CTCCTCTGCCACTGGTCCCAAGGGACCGGGGTCATCAGTTTCATCACACGTA |
| | | CACACGTAGTGCATCCCCGACCAGCCCCAAGGTCTTCCCGCTGAGCC | | GTGCATCCCCGACCAGCCCCAAGGTCTTCCCGCTGAGCCTCTGCAGCACCCCO |
| | | TCTGCAGCACCCCCCAAGATGGGAACGTGGTCGTCGCATGCCTGGTC | | CAAGATGGGAACGTGGTCGTCGCATGCCTGGTCCAGGGCTTCTTCCCCC |
| | | CAGGGCTTCTTCCCCCATTCGGAGACCTCCCGCTACTCTGGCGTCGAT | | |
| | | GAGGGA | | |
| Human- | 77 | CAATCCGCCCTCACTACAACCGGATACGCCGGTCTCGAATGTAATAC | 78 | GGCACGTAGTAGAGCGTGTCATTATGTACCGCCACAGGCTCCGGAAGTCCG |
| IGHA14_ | | GACTCACTATAGGCACGTAGTAGAGCGTGTCATTATGTACCGCCAC | | ACGAGGTGACGTCCATCCGCCACGACTACCTGAACAGCCGACTCTACCATT |
| rev | | AGGCTCCGGAAGTCCGACGAGGTGACGTCCATCCGCCACGACTACC | | GGACCGGAACCTTCCTGCCCGACATAGACCATAGTCTCAGTGGTCCTATCTA |
| | | TGAACAGCCGACTCTACCATTGGACCGGAACCTTCCTGCCCGACATA | | CTAGGGGTAGGTGAGGTCCGGAAAGGGCCCGTAGACCGCGTGGGTCGGCT |
| | | GACCATAGTCTCAGTGGTCCTATCTACTAGGGGTAGGTGAGGTCCG | | AGGTCATCGACCATTTCGACATAGGTCTTGGGAATGTCCTCTAGAAGTCTC |
| | | GAAAGGGCCCGTAGACCGCGTGGGTCGGCTAGGTCATCGACCATTT | | TGAGGGGGCCGAAAAAGTGGAGACGAGGTCTGACGTGGTCGACGTGGAG |
| | | CGACATAGGTCTTGGGAATGTCCTCTAGAAGTCTCTGAGGGGGCCG | | CGCCCCAGCAAGCAATATTATTGAGGGGCTTGTATCATTATGGCGGGGCA |
| | | AAAAAGTGGAGACGAGGTCTGACGTGGTCGACGTGGAGCGCCCCA | | GAGACTCCTCTGCCACTGGCACCAGGGAACCGGGGTCTGCAGGTATGGCAT |
| | | GCAAGCAATATTATTGAGGGGCTTGTATCATTATGGCGGGGCAGA | | CATCATCATCATTACACGTAGTGCATCCCCGACCAGCCCCAAGGTCTTCCCG |
| | | GACTCCTCTGCCACTGGCACCAGGGAACCGGGGTCTGCAGGTATGG | | CTGAGCCTCTGCAGCACCCCCCAAGATGGGAACGTGGTCGTCGCATGCCTG |
| | | CATCATCATCATCATTACACGTAGTGCATCCCCGACCAGCCCCAAGG | | GTCCAGGGCTTCTTCCCCC |
| | | TCTTCCCGCTGAGCCTCTGCAGCACCCCCCAAGATGGGAACGTGGTC | | |
| | | GTCGCATGCCTGGTCCAGGGCTTCTTCCCCCATTCGGAGACCTCCCGC | | |
| | | TACTCTGGCGTCGATGAGGGA | | |
| Human- | 79 | CAATCCGCCCTCACTACAACCGGATACGCCGGTCTCGAATGTAATAC | 80 | GGCACGTAGTAGAGAGCGTGTCATTATGTGCCGGCACAGGAGTCTAGAGT |
| IGHA15_ | | GACTCACTATAGGCACGTAGTAGAGAGCGTGTCATTATGTGCCGGC | | CCGACGAGTCGAGGTACATCCGACACGAGCACCTAAACAGGCGCCATTAG |
| rev | | ACAGGAGTCTAGAGTCCGACGAGTCGAGGTACATCCGACACGAGC | | CACTGAGACGGGACCTTGAAGACACGCATCAAACGACATGGTTTCTATCCC |
| | | ACCTAAACAGGCGCCATTAGCACTGAGACGGGACCTTGAAGACAC | | TACTAGGGAGGGTAGGTGAGTTCGGGAACAGGTCCCCGGACAGCGTGGG |
| | | GCATCAAACGACATGGTTTCTATCCCTACTAGGGAGGGTAGGTGAG | | TCGACTATCGTATCGACGACTTCCACGGAGGTCTTCGGAACGTCCTCTGGA |
| | | TTCGGGAACAGGTCCCCGGACAGCGTGGGTCGACTATCGTATCGAC | | AGTGGCTCCTGGGTCCGAAGAAGTGGAGTCGGGGTCTGACGTGGTCGACC |
| | | GACTTCCACGGAGGTCTTCGGAACGTCCTCTGGAAGTGGCTCCTGG | | TGGACAGCAACGACGCACATCAAGGTCAATATGGAACAGACTTCTCTGCC |
| | | GTCCGAAGAAGTGGAGTCGGGGTCTGACGTGGTCGACCTGGACAG | | ACTGGTAACAGGGAACCGGGGTCTGTAGTTTTCGTAGTCACGTAGTGCATC |
| | | CAACGACGCACATCAAGGTCAATATGGAACAGACTTCTCTGCCACT | | CCCGACCAGCCCCAAGGTCTTCCCGCTGAGCCTCTGCAGCACCCCCCAAGAT |
| | | GGTAACAGGGAACCGGGGTCTGTAGTTTTCGTAGTCACGTAGTGCA | | GGGAACGTGGTCGTCGCATGCCTGGTCCAGGGCTTCTTCCCCC |
| | | TCCCCGACCAGCCCCAAGGTCTTCCCGCTGAGCCTCTGCAGCACCCCC | | |
| | | CAAGATGGGAACGTGGTCGTCGCATGCCTGGTCCAGGGCTTCTTCCC | | |
| | | CCATTCGGAGACCTCCCGCTACTCTGGCGTCGATGAGGGA | | |
| Human- | 81 | CAATCCGCCCTCACTACAACCGGATACGCCGGTCTCGAATGTAATAC | 82 | GGCACGTAGTGTCATTATGTGCCGGCACAGGCGCCGCCAGTGTCTCGGGTC |
| IGHE6_ | | GACTCACTATAGGCACGTAGTGTCATTATGTGCCGGCACAGGCGCC | | GAAGTCCCTCTTGACCAAGAACCTGCACAGATGACTATACCACTGAGCTGA |
| rev | | GCCAGTGTCTCGGGTCGAAGTCCCTCTTGACCAAGAACCTGCACAG | | GAACTCCCTGCCCAACATCAACCACGAAGGTGATACTAACTAAAGGGGTT |
| | | ATGACTATACCACTGAGCTGAGAACTCCCTGCCCAACATCAACCACG | | AGGTGAGGTCGGGGAAGGGACCCCCGACCGCCTAGGTCGAGGTCATCATT |
| | | AAGGTGATACTAACTAAAGGGGTTAGGTGAGGTCGGGGAAGGGA | | GGTGACTTCCTGGGTGGTATCTGTCGCGTCCACTCCCTGTCCCAGAGGCTTC |
| | | CCCCCGACCGCCTAGGTCGAGGTCATCATTGGTGACTTCCTGGGTGG | | CGAAGTTGTCAGGACGCGGGGTGACGACATCGACGTGGACAGAGGCAGC |
| | | TATCTGTCGCGTCCACTCCCTGTCCCAGAGGCTTCCGAAGTTGTCAG | | GACGCCATCAATGACATCAGTAGAGCACGGAAGACTTCTCTGCCACTGGTA |
| | | GACGCGGGGTGACGACATCGACGTGGACAGAGGCAGCGACGCCAT | | ACAGGGAACCGGGGTCTGTAGTTTTCGTAGTCACGTAGTGCCTCCACACAG |
| | | CAATGACATCAGTAGAGCACGGAAGACTTCTCTGCCACTGGTAACA | | AGCCCATCCGTCTTCCCCTTGACCCGCTGCTGCAAAAACATTCCCTCCAATGC |
| | | GGGAACCGGGGTCTGTAGTTTTCGTAGTCACGTAGTGCCTCCACAC | | CACCTCCGTGACTCTGGGCTGCCTGGCCACGGGCT |
| | | AGAGCCCATCCGTCTTCCCCTTGACCCGCTGCTGCAAAAACATTCCCT | | |
| | | CCAATGCCACCTCCGTGACTCTGGGCTGCCTGGCCACGGGCTATTCG | | |
| | | GAGACCTCCCGCTACTCTGGCGTCGATGAGGGA | | |
| Human- | 83 | CAATCCGCCCTCACTACAACCGGATACGCCGGTCTCGAATGTAATAC | 84 | GGCACGTAGTGAGAGCGTGTCATTATGTGTCGGCACAGAAGTCTAGAGTC |
| IGHE11_ | | GACTCACTATAGGCACGTAGTGAGAGCGTGTCATTATGTGTCGGCA | | CGACGAGTCGAGGTACATCCGACACGAGCGCCTACACAGGGACCATTACC |
| rev | | CAGAAGTCTAGAGTCCGACGAGTCGAGGTACATCCGACACGAGCG | | ACTGAGACGGGACCTTGAAGACACTTATAAAACACAATGGTAACGGTCAC |
| | | CCTACACAGGGACCATTACCACTGAGACGGGACCTTGAAGACACTT | | AACTAGGTAGGGTAGGTGAGTTCGGAAACAGGCCCCCGGACCGCGTGGGT |
| | | ATAAAACACAATGGTAACGGTCACAACTAGGTAGGGTAGGTGAGT | | TACGTATCGTATCGATCACTTCCACATAGGTCTTCGGAACGTCCTTTGGAAG |
| | | TCGGAAACAGGCCCCCGGACCGCGTGGGTTACGTATCGTATCGATC | | TGACTCCGGGGTCCGAAGAAGTGGAGTCGGGGTCTGACGTGTTCGACCTG |
| | | ACTTCCACATAGGTCTTCGGAACGTCCTTTGGAAGTGACTCCGGGGT | | GACAACGACAACAAAAACACCACAGAAACAGAGAGAGAGAAGAACAAG |
| | | CCGAAGAAGTGGAGTCGGGGTCTGACGTGTTCGACCTGGACAACG | | GAGAAAGAGGAAGAGGAAGAAGAAGAGAAGAGGAGGAGAAGGCAAG |
| | | ACAACAAAAACACCACAGAAACAGAGAGAGAGAAGAACAAGGA | | CAGACTCCTCTGCCACTGGTCCCAAGGGACCGGGGTCATCAGTTTCATCACA |
| | | GAAAGAGGAAGAGGAAGAAGAAGAGAAGAGGAGGAGAAGGCA | | CGTAGTGCCTCCACACAGAGCCCATCCGTCTTCCCCTTGACCCGCTGCTGCAA |
| | | AGCAGACTCCTCTGCCACTGGTCCCAAGGGACCGGGGTCATCAGTTT | | AAACATTCCCTCCAATGCCACCTCCGTGACTCTGGGCTGCCTGGCCACGGGC |
| | | CATCACACGTAGTGCCTCCACACAGAGCCCATCCGTCTTCCCCTTGAC | | T |
| | | CCGCTGCTGCAAAAACATTCCCTCCAATGCCACCTCCGTGACTCTGG | | |
| | | GCTGCCTGGCCACGGGCTATTCGGAGACCTCCCGCTACTCTGGCGTC | | |
| | | GATGAGGGA | | |
| Human- | 85 | CAATCCGCCCTCACTACAACCGGATACGCCGGTCTCGAATGTAATAC | 86 | GGCACGTAGTAGAGAGCGTGTCATTATGTGCCGGCACAGGAGTCTAGAGT |
| IGHE13_ | | GACTCACTATAGGCACGTAGTAGAGAGCGTGTCATTATGTGCCGGC | | CCGACGAGTCGAGGTACATCCGACACGAGCACCTAAGCAGGCGCCATTAG |
| rev | | ACAGGAGTCTAGAGTCCGACGAGTCGAGGTACATCCGACACGAGC | | CACTGAGACGGGACCTTGAAGACACGCATCAAACGACATGGTTCCTATCCC |
| | | ACCTAAGCAGGCGCCATTAGCACTGAGACGGGACCTTGAAGACAC | | TACTAGGAAGGGTAGGTGAGTTCGGGAACAGGTCCCCGGACAGCGTGGG |
| | | GCATCAAACGACATGGTTCCTATCCCTACTAGGAAGGGTAGGTGAG | | TCGACTATCGTATCGACGACTTCCACGGAGGTCTTCGGAACGTCCTCTGGA |
| | | TTCGGGAACAGGTCCCCGGACAGCGTGGGTCGACTATCGTATCGAC | | AGTGGCTCCTGGGTCCGAAGAAGTGGAGTCGGGGTCTGACGTGGTCGACC |
| | | GACTTCCACGGAGGTCTTCGGAACGTCCTCTGGAAGTGGCTCCTGG | | |
| | | GTCCGAAGAAGTGGAGTCGGGGTCTGACGTGGTCGACCTGGACAG | | TGGACAGGAGCGGCCGCCATCAATCGTGGTATCAGTAACCAAGACTCCTCT |
| | | GAGCGGCCGCCATCAATCGTGGTATCAGTAACCAAGACTCCTCTGCC | | GCCACTGGCACCAGGGAACCGGGGTCTGCAGGTATGGCATCATCATCATCA |
| | | ACTGGCACCAGGGAACCGGGGTCTGCAGGTATGGCATCATCATCAT | | TTACACGTAGTGCCTCCACACAGAGCCCATCCGTCTTCCCCTTGACCCGCTGC |
| | | CATTACACGTAGTGCCTCCACACAGAGCCCATCCGTCTTCCCCTTGAC | | TGCAAAAACATTCCCTCCAATGCCACCTCCGTGACTCTGGGCTGCCTGGCCA |
| | | CCGCTGCTGCAAAAACATTCCCTCCAATGCCACCTCCGTGACTCTGG | | CGGGCT |
| | | GCTGCCTGGCCACGGGCTATTCGGAGACCTCCCGCTACTCTGGCGTC | | |
| | | GATGAGGGA | | |
| Human- | 87 | CAATCCGCCCTCACTACAACCGGATACGCCGGTCTCGAATGTAATAC | 88 | GGCACGTAGTAGAGAGCGTGTCATTATGTGCCGGCACAGGAGTCTAGAGT |
| IGHG1_ | | GACTCACTATAGGCACGTAGTAGAGAGCGTGTCATTATGTGCCGGC | | CCGACGAGTCGAGGTACATCCGACACGAGCACCTAAACAGGCGCCATTAG |
| rev | | ACAGGAGTCTAGAGTCCGACGAGTCGAGGTACATCCGACACGAGC | | CACTGAGACGGGACCTTGAAGACACGCATCAAACGATATGGTTTCTATCCC |
| | | ACCTAAACAGGCGCCATTAGCACTGAGACGGGACCTTGAAGACAC | | TACTAGGAAGGGTAGGTGAGTTCGGGAACAGGTCCCCGGACAGCGTGGG |
| | | GCATCAAACGATATGGTTTCTATCCCTACTAGGAAGGGTAGGTGAG | | TCGACTATCGTATCGACGACTTCCACGGAGGTCTTCGGAACGTCCTCTGGA |
| | | TTCGGGAACAGGTCCCCGGACAGCGTGGGTCGACTATCGTATCGAC | | |
| | | GACTTCCACGGAGGTCTTCGGAACGTCCTCTGGAAGTGGCTCCTGG | | |
| | | GTCCGAAGAAGTGGAGTCGGGGTCTGACGTGGTCGACCTGGACGG | | AGTGGCTCCTGGGTCCGAAGAAGTGGAGTCGGGGTCTGACGTGGTCGACC |
| | | CGACGCCGTATCGTCGACCATGATGATGTTATAGGAGGCCGACTCC | | TGGACGGCGACGCCGTATCGTCGACCATGATGATGTTATAGGAGGCCGAC |
| | | TCTGCCACTGGTCCCAAGGAACCGGGGTCATCAGTTTCATCACACGT | | TCCTCTGCCACTGGTCCCAAGGAACCGGGGTCATCAGTTTCATCACACGTAG |
| | | AGTGCCTCCACCAAGGGCCCATCGGTCTTCCCCCTGGTGCCCTCCTCC | | TGCCTCCACCAAGGGCCCATCGGTCTTCCCCCTGGTGCCCTCCTCCAGGAGCG |
| | | AGGAGCGTCTCTGAGGGCACAGCGGCCCTGGGCTGCCTGGTCAAGG | | TCTCTGAGGGCACAGCGGCCCTGGGCTGCCTGGTCAAGGACTACTTCC |
| | | ACTACTTCCATTCGGAGACCTCCCGCTACTCTGGCGTCGATGAGGGA | | |
| Human- | 89 | CAATCCGCCCTCACTACAACCGGATACGCCGGTCTCGAATGTAATAC | 90 | GGCACGTAGTAGAGAGCGTGTCATTATGTGTCGGTACAGGAGTCTAGAGT |
| IGHG2_ | | GACTCACTATAGGCACGTAGTAGAGAGCGTGTCATTATGTGTCGGT | | CCGACGAGTCGAGGTACATCCGACACGAGCGCCTACACAGGGACCATTACC |
| rev | | ACAGGAGTCTAGAGTCCGACGAGTCGAGGTACATCCGACACGAGC | | ACTGAGACGGGACCTTGAGGACACTTATAAAACACAATGGTAACGGTCGC |
| | | GCCTACACAGGGACCATTACCACTGAGACGGGACCTTGAGGACACT | | AACTAGGTAGGGTAGGTGAGTTCGGAAACAGGCCCCCGGACCGCGTGGGT |
| | | TATAAAACACAATGGTAACGGTCGCAACTAGGTAGGGTAGGTGAG | | TACGTATCGTATCGATCACTTCCACATAGGTCTTCGGAACGTCCTTTGGAAG |
| | | TTCGGAAACAGGCCCCCGGACCGCGTGGGTTACGTATCGTATCGAT | | TGACTCCGGGGTCCGAAGAAGTGGAGTCGGGGTCTGACGTGGTCGACCTG |
| | | CACTTCCACATAGGTCTTCGGAACGTCCTTTGGAAGTGACTCCGGGG | | GACCAGCAGCAGCCGACCACAGAAACAGAGAGAGAGAAGAACAAGGAG |
| | | TCCGAAGAAGTGGAGTCGGGGTCTGACGTGGTCGACCTGGACCAG | | AAAGAGGAAGAGGAAGAAGAAGAGAAGAGGAGGAGAAGGAGAAGGA |
| | | CAGCAGCCGACCACAGAAACAGAGAGAGAGAAGAACAAGGAGA | | CTTCTCTGCCACTGGTAACAGGGAACCGGGGTCTATAGTTTTCGTAGTCAC |
| | | AAGAGGAAGAGGAAGAAGAAGAGAAGAGGAGGAGAAGGAGAA | | GTAGTGCCTCCACCAAGGGCCCATCGGTCTTCCCCCTGGTGCCCTCCTCCAGG |
| | | GGACTTCTCTGCCACTGGTAACAGGGAACCGGGGTCTATAGTTTTC | | AGCGTCTCTGAGGGCACAGCGGCCCTGGGCTGCCTGGTCAAGGACTACTTC |
| | | GTAGTCACGTAGTGCCTCCACCAAGGGCCCATCGGTCTTCCCCCTGG | | C |
| | | TGCCCTCCTCCAGGAGCGTCTCTGAGGGCACAGCGGCCCTGGGCTGC | | |
| | | CTGGTCAAGGACTACTTCCATTCGGAGACCTCCCGCTACTCTGGCGT | | |
| | | CGATGAGGGA | | |
| Human- | 91 | CAATCCGCCCTCACTACAACCGGATACGCCGGTCTCGAATGTAATAC | 92 | GGCACGTAGTAGAGAGCGTGTCATTATGTGCCGGCACAGGAGTCTAGAGT |
| IGHG3_ | | GACTCACTATAGGCACGTAGTAGAGAGCGTGTCATTATGTGCCGGC | | CCGACGAGTCGAGGTACATCCGACACGAGCACCTAAACAGGCGCCATTAG |
| rev | | ACAGGAGTCTAGAGTCCGACGAGTCGAGGTACATCCGACACGAGC | | CACTGAGACGGGACCTTGAAGACACGCATCAAACGATATGGTTTCTATCCC |
| | | ACCTAAACAGGCGCCATTAGCACTGAGACGGGACCTTGAAGACAC | | TACTAGGGAGGGTAGGTGAGTTCGGGAACAGGTCCCCGGACAGCGTGGG |
| | | GCATCAAACGATATGGTTTCTATCCCTACTAGGGAGGGTAGGTGAG | | TCGACTATCGTATCGACGACTTCCACGGAGGTCTTCGGAACGTCCTCTGGA |
| | | TTCGGGAACAGGTCCCCGGACAGCGTGGGTCGACTATCGTATCGAC | | AGTGGCTCCTGGGTCCGAAGAAGTGGAGTCGGGGTCTGACGTGGTCGACG |
| | | GACTTCCACGGAGGTCTTCGGAACGTCCTCTGGAAGTGGCTCCTGG | | TGGACCGCCCAACAAGGGCATTAGCATCTGTGATATAGGTGGCACGGAAA |
| | | GTCCGAAGAAGTGGAGTCGGGGTCTGACGTGGTCGACGTGGACCG | | GAGACTCCTCTGCCACTGGCACCAGGGAACGGGGGTCTGCAGGTATGGCA |
| | | CCCAACAAGGGCATTAGCATCTGTGATATAGGTGGCACGGAAAGA | | TCATCATCATCATTACACGTAGTGCCTCCACCAAGGGCCCATCGGTCTTCCC |
| | | GACTCCTCTGCCACTGGCACCAGGGAACGGGGGTCTGCAGGTATGG | | C |
| | | CATCATCATCATCATTACACGTAGTGCCTCCACCAAGGGCCCATCGG | | CTGGTGCCCTCCTCCAGGAGCGTCTCTGAGGGCACAGCGGCCCTGGGCTGC |
| | | TCTTCCCCCTGGTGCCCTCCTCCAGGAGCGTCTCTGAGGGCACAGCG | | C TGGTCAAGGACTACTTCC |
| | | GCCCTGGGCTGCCTGGTCAAGGACTACTTCCATTCGGAGACCTCCCG | | |
| | | CTACTCTGGCGTCGATGAGGGA | | |
| Human- | 93 | CAATCCGCCCTCACTACAACCGGATACGCCGGTCTCGAATGTAATAC | 94 | GGCACGTAGTCCTCCATTCAATACTAGGACGACTGTCATTATTTGACGTTTT |
| IGK1_ | | GACTCACTATAGGCACGTAGTCCTCCATTCAATACTAGGACGACTGT | | AGAAGTCCGACGTCCGACGACTACCACTCTCACTTCAGAGAGGGTCTGGGT |
| rev | | CATTATTTGACGTTTTAGAAGTCCGACGTCCGACGACTACCACTCTC | | GACGGTGACTTGGACCGACCCTACGATCACCGGGACCACCTACGTGGTATC |
| | | ACTTCAGAGAGGGTCTGGGTGACGGTGACTTGGACCGACCCTACGA | | TACTCCTCGGACCCGCGGACCGGTCCAAAGACGACTATGGTCCAATTCATC |
| | | TCACCGGGACCACCTACGTGGTATCTACTCCTCGGACCCGCGGACCG | | GACGACGATTGTGAGACTGACCGGGACGTCCTCTCCCACTGAGAAAGGGG |
| | | GTCCAAAGACGACTATGGTCCAATTCATCGACGACGATTGTGAGAC | | ACCTCTGTTTCTGTCCCACCCACCTCTGACACAGTAATGTTAAAGAGAACAC |
| | | TGACCGGGACGTCCTCTCCCACTGAGAAAGGGGACCTCTGTTTCTGT | | GCCAACGCGGAGCAAAGCAAACTAAAGGTGGAACCAGGGAACCGGCTTG |
| | | CCCACCCACCTCTGACACAGTAATGTTAAAGAGAACACGCCAACGC | | CAGGTGCACGTAGTCGAACTGTGGCTGCACCATCTGTCTTCATCTTCCCGCC |
| | | GGAGCAAAGCAAACTAAAGGTGGAACCAGGGAACCGGCTTGCAG | | ATCTGATGAGCAGTTGAAATCTGGAACTGCCTCTGTTGTGTG |
| | | GTGCACGTAGTCGAACTGTGGCTGCACCATCTGTCTTCATCTTCCCGC | | |
| | | CATCTGATGAGCAGTTGAAATCTGGAACTGCCTCTGTTGTGTGATTC | | |
| | | GGAGACCTCCCGCTACTCTGGCGTCGATGAGGGA | | |
| Human- | 95 | CAATCCGCCCTCACTACAACCGGATACGCCGGTCTCGAATGTAATAC | 96 | GGCACGTAGTACTCCATTTGATGATGAGACTACTGTCATTATGCAACGTCG |
| IGK3_ | | GACTCACTATAGGCACGTAGTACTCCATTTGATGATGAGACTACTG | | TAGAAGTCGAAGGTCCGATAACTACCACTCCCACTTTAGACAGGGTCTAGG |
| rev | | TCATTATGCAACGTCGTAGAAGTCGAAGGTCCGATAACTACCACTC | | TGACGGTGACTTGGAGCTCCCCTGGGGACTCTACCTGACCCTTCGTATGAA |
| | | CCACTTTAGACAGGGTCTAGGTGACGGTGACTTGGAGCTCCCCTGG | | C |
| | | GGACTCTACCTGACCCTTCGTATGAACTACTCCTCGAAACCTCTGACT | | TACTCCTCGAAACCTCTGACTAGACCAAAGACGACCATGGTCACATTCGAT |
| | | AGACCAAAGACGACCATGGTCACATTCGATGATGGTTACGAGACT | | GATGGTTACGAGACTGACCGGGCCGTCCACTACCACTGAAAGAGGAAACC |
| | | GACCGGGCCGTCCACTACCACTGAAAGAGGAAACCTCAGTGTCTGA | | TCAGTGTCTGACTTTCAGACCTCTGACTCAGTCGTGTTAAAGGCACCACCGC |
| | | CTTTCAGACCTCTGACTCAGTCGTGTTAAAGGCACCACCGCAGCAGG | | AGCAGGCGCAAACTAGAGGTCGAACCAGGGGACCGGTTTTGACATGTCAC |
| | | CGCAAACTAGAGGTCGAACCAGGGGACCGGTTTTGACATGTCACGT | | GTAGTCGAACTGTGGCTGCACCATCTGTCTTCATCTTCCCGCCATCTGATGA |
| | | AGTCGAACTGTGGCTGCACCATCTGTCTTCATCTTCCCGCCATCTGAT | | GCAGTTGAAATCTGGAACTGCCTCTGTTGTGTG |
| | | GAGCAGTTGAAATCTGGAACTGCCTCTGTTGTGTGATTCGGAGACC | | |
| | | TCCCGCTACTCTGGCGTCGATGAGGGA | | |
| Human- | 97 | CAATCCGCCCTCACTACAACCGGATACGCCGGTCTCGAATGTAATAC | 98 | GGCACGTAGTCCTACGGTCAACGATGCGACGACTGTCATTATGTGACGTTT |
| IGK7_ | | GACTCACTATAGGCACGTAGTCCTACGGTCAACGATGCGACGACTG | | TAGAAGTCCGAGATCCGACGACTACCACTCTCACTTCAGACAGGGTCCGGG |
| rev | | TCATTATGTGACGTTTTAGAAGTCCGAGATCCGACGACTACCACTCT | | TGACGGTGACTTGGACCGACCCTACGGTCACCGGGACAACCTACGTAGTAT |
| | | CACTTCAGACAGGGTCCGGGTGACGGTGACTTGGACCGACCCTACG | | CTACTCCTCGGACCCTCGGACCGGTCCAAAGACGACCATGGTCCGATTCAAC |
| | | GTCACCGGGACAACCTACGTAGTATCTACTCCTCGGACCCTCGGACC | | GACGATTGTGGGACTGACCGGGACGTCCTCTCCCACCGAGAAAGGGGACC |
| | | GGTCCAAAGACGACCATGGTCCGATTCAACGACGATTGTGGGACTG | | TCTGTTTCTGTCCCACCGACCTCTGACACAGTTGTGTTAAAGGACAGCCGCA |
| | | ACCGGGACGTCCTCTCCCACCGAGAAAGGGGACCTCTGTTTCTGTCC | | CGCCCAAATTAGAGGTCAGCACAGGGAACCGGCTTCCACTAGCACGTAGTC |
| | | CACCGACCTCTGACACAGTTGTGTTAAAGGACAGCCGCACGCCCAA | | GAACTGTGGCTGCACCATCTGTCTTCATCTTCCCGCCATCTGATGAGCAGTT |
| | | ATTAGAGGTCAGCACAGGGAACCGGCTTCCACTAGCACGTAGTCGA | | GAAATCTGGAACTGCCTCTGTTGTGTG |
| | | ACTGTGGCTGCACCATCTGTCTTCATCTTCCCGCCATCTGATGAGCAG | | |
| | | TTGAAATCTGGAACTGCCTCTGTTGTGTGATTCGGAGACCTCCCGCT | | |
| | | ACTCTGGCGTCGATGAGGGA | | |
| Human- | 99 | CAATCCGCCCTCACTACAACCGGATACGCCGGTCTCGAATGTAATAC | 100 | GGCACGTAGTACTCGTGACTCCGACGACAGGGTACGACTCGTCATTATCAG |
| IGL4_ | | GACTCACTATAGGCACGTAGTACTCGTGACTCCGACGACAGGGTAC | | TCGGAGCAGGAGTCCGACCTCAGGTCATTACCAGTCCCTCCGACACAAAGG |
| rev | | GACTCGTCATTATCAGTCGGAGCAGGAGTCCGACCTCAGGTCATTA | | ACTGGACCTACGTCTCTTAGAGAGACTCTAGGGACTCCCGGCCAACAATAA |
| | | CCAGTCCCTCCGACACAAAGGACTGGACCTACGTCTCTTAGAGAGA | | GGACATCCTATCCTCAAACCCTCCCACCGGGACCACGACGACGTCGGTTCGA |
| | | CTCTAGGGACTCCCGGCCAACAATAAGGACATCCTATCCTCAAACCC | | CGAGGAACCAACGGTTGTTACAACGACAAGGGTCACGTCCACTCACACCGC |
| | | TCCCACCGGGACCACGACGACGTCGGTTCGACGAGGAACCAACGGT | | CAGACAGAGTTCGGGAACCTGTGGCTCCCACCGACTCAGTCGGGACGGAC |
| | | TGTTACAACGACAAGGGTCACGTCCACTCACACCGCCAGACAGAGT | | GAGCAGAACACACAGCACCGCTCCTGCCAGTCGACCCACGGAGGAGGCTT |
| | | TCGGGAACCTGTGGCTCCCACCGACTCAGTCGGGACGGACGAGCAG | | GTGTCGTCACGTAGTGGTCAGCCCAAGGCTGCCCCCTCGGTCACTCTGTTCC |
| | | AACACACAGCACCGCTCCTGCCAGTCGACCCACGGAGGAGGCTTGT | | CGCCCTCCTCTGAGGAGCTTCAAGCCAACAAGGCCACACTGGTGTGTCTCAT |
| | | GTCGTCACGTAGTGGTCAGCCCAAGGCTGCCCCCTCGGTCACTCTGT | | A |
| | | TCCCGCCCTCCTCTGAGGAGCTTCAAGCCAACAAGGCCACACTGGTG | | |
| | | TGTCTCATAATTCGGAGACCTCCCGCTACTCTGGCGTCGATGAGGGA | | |
| Human- | 101 | CAATCCGCCCTCACTACAACCGGATACGCCGGTCTCGAATGTAATAC | 102 | A |
| IGL5_ | | GACTCACTATAGGCACGTAGTACTCGTGACTCCGACGACAGGGTAC | | GGCACGTAGTACTCGTGACTCCGACGACAGGGTACGACTCGTCATTATCAG |
| rev | | GACTCGTCATTATCAGTCGGAGCAGGAGTCCGACCTCAGGTCATTA | | TCGGAGCAGGAGTCCGACCTCAGGTCATTACCAGTCCCTCCGACACAAAGG |
| | | CCAGTCCCTCCGACACAAAGGACTGGACCTACGTCTCTTAGAGAGA | | ACTGGACCTACGTCTCTTAGAGAGACTCTAGGGACTCCCGGCCAACAATAA |
| | | CTCTAGGGACTCCCGGCCAACAATAAGGACATCCTATCCTCAAACCC | | GGACATCCTATCCTCAAACCCTCCCACCGGGACCACGACGACGTCGGTTCGA |
| | | TCCCACCGGGACCACGACGACGTCGGTTCGACGAGGAACCAACGGT | | CGAGGAACCAACGGTCGTAACAACGACAAGGGTCACGTCCACTCACACCG |
| | | CGTAACAACGACAAGGGTCACGTCCACTCACACCGCCAGACAGAGT | | CCAGACAGAGTTCGGGAACCTGTGGCTCCCACCGACTCAGTCGGGACGGAC |
| | | TCGGGAACCTGTGGCTCCCACCGACTCAGTCGGGACGGACGGAGAA | | GGAGAAGCCAGGCGAAGCGAATTCTGTCACCCGACCCAGAACGACGGTTT |
| | | GCCAGGCGAAGCGAATTCTGTCACCCGACCCAGAACGACGGTTTGT | | GTACACGCACGTAGTGGTCAGCCCAAGGCTGCCCCCTCGGTCACTCTGTTCC |
| | | ACACGCACGTAGTGGTCAGCCCAAGGCTGCCCCCTCGGTCACTCTGT | | CGCCCTCCTCTGAGGAGCTTCAAGCCAACAAGGCCACACTGGTGTGTCTCAT |
| | | TCCCGCCCTCCTCTGAGGAGCTTCAAGCCAACAAGGCCACACTGGTG | | |
| | | TGTCTCATAATTCGGAGACCTCCCGCTACTCTGGCGTCGATGAGGGA | | |
| Human- | 103 | CAATCCGCCCTCACTACAACCGGATACGCCGGTCTCGAATGTAATAC | 104 | GGCACGTAGTACTCGTGACTCCGACGACAGGTTACGACTCGTCATTATCAG |
| IGL7_ | | GACTCACTATAGGCACGTAGTACTCGTGACTCCGACGACAGGTTAC | | TCGGAGCAGGAGTCCGACCTCAGGTCATTACCAGTCCCTCCGACACAAAGG |
| rev | | GACTCGTCATTATCAGTCGGAGCAGGAGTCCGACCTCAGGTCATTA | | ACTGGACCTACGTCTCTTAGAGAGACTCTAGGGACTCCCGGCCAACAATAA |
| | | CCAGTCCCTCCGACACAAAGGACTGGACCTACGTCTCTTAGAGAGA | | GGACATCCTATCCTCAAACCCTCCCACCGGGACCACGACGACGTCGGTTCGA |
| | | CTCTAGGGACTCCCGGCCAACAATAAGGACATCCTATCCTCAAACCC | | CGAGGAACCAACGGTTGTTACAACGACAAGGGTCACGTCCACTCACACCGC |
| | | TCCCACCGGGACCACGACGACGTCGGTTCGACGAGGAACCAACGGT | | CAGACAGAGTTCGGGAACCTGTGGCTCCCACCGACTCAGTCGGGACGGACC |
| | | TGTTACAACGACAAGGGTCACGTCCACTCACACCGCCAGACAGAGT | | GCGGGAGGGAGGCGACACGGGATTTTACTAGTCGACCCAAGGAGGTGGT |
| | | TCGGGAACCTGTGGCTCCCACCGACTCAGTCGGGACGGACCGCGGG | | TTATGTTTTCACGTAGTGGTCAGCCCAAGGCTGCCCCCTCGGTCACTCTGTTC |
| | | AGGGAGGCGACACGGGATTTTACTAGTCGACCCAAGGAGGTGGTT | | CCGCCCTCCTCTGAGGAGCTTCAAGCCAACAAGGCCACACTGGTGTGTCTCA |
| | | TATGTTTTCACGTAGTGGTCAGCCCAAGGCTGCCCCCTCGGTCACTCT | | TA |
| | | GTTCCCGCCCTCCTCTGAGGAGCTTCAAGCCAACAAGGCCACACTGG | | |
| | | TGTGTCTCATAATTCGGAGACCTCCCGCTACTCTGGCGTCGATGAGG GA | | |
| Human- | 105 | CAATCCGCCCTCACTACAACCGGATACGCCGGTCTCGAATGTAATAC | 106 | GGCACGTAGTGCGAACGACGTGTCTTCATCCACCGACTCAGAGGTCCGACC |
| TRA1_ | | GACTCACTATAGGCACGTAGTGCGAACGACGTGTCTTCATCCACCG | | CTTAGGTACTATACGTTACTCTTGACGAACCGTGAAAATAACTTCCTCTAAC |
| rev | | ACTCAGAGGTCCGACCCTTAGGTACTATACGTTACTCTTGACGAACC | | ACTTAGAAGGAAGAAAGAAAAGTGAGTGTAGACCTGCATACCGATAGTT |
| | | GTGAAAATAACTTCCTCTAACACTTAGAAGGAAGAAAGAAAAGTG | | ATTACGTCCCGGAAAGGGTCCCTTAACAACCATGGTACCTTTCATCAGTTTG |
| | | AGTGTAGACCTGCATACCGATAGTTATTACGTCCCGGAAAGGGTCC | | CGTCACAAGAGTATTCGTGTCAAATATTAACCTTAGGGAGGAAAGACCTG |
| | | CTTAACAACCATGGTACCTTTCATCAGTTTGCGTCACAAGAGTATTC | | ATAGTTTCTAACTCCTGAAACAAAGTGGACGACGACCGAGCACGAAAAAC |
| | | GTGTCAAATATTAACCTTAGGGAGGAAAGACCTGATAGTTTCTAAC | | AACGAAAGCCAACCTAAATAAAAACATGAGTAGGGGAAAGGGGTTGTCC |
| | | TCCTGAAACAAAGTGGACGACGACCGAGCACGAAAAACAACGAA | | TCGTAATGGTCGTGAACACACGTAGTATATCCAGAACCCTGACCCTGCCGT |
| | | AGCCAACCTAAATAAAAACATGAGTAGGGGAAAGGGGTTGTCCTC | | GTACCAGCTGAGAGACTCTAAATCCAGTGACAAGTCTGTCTGCCTATTCAC |
| | | GTAATGGTCGTGAACACACGTAGTATATCCAGAACCCTGACCCTGC | | CGATTTTGATT |
| | | CGTGTACCAGCTGAGAGACTCTAAATCCAGTGACAAGTCTGTCTGC | | |
| | | CTATTCACCGATTTTGATTATTCGGAGACCTCCCGCTACTCTGGCGTC | | |
| | | GATGAGGGA | | |
| Human- | 107 | CAATCCGCCCTCACTACAACCGGATACGCCGGTCTCGAATGTAATAC | 108 | GGCACGTAGTACGAACGACGTGTCTTCATCCACCGACTCAGAGGTCCGACC |
| TRA4_ | | GACTCACTATAGGCACGTAGTACGAACGACGTGTCTTCATCCACCG | | CTTAGGTACTATACGCTACTCTTGACGAACCGTGAAAATAACTTCCTCTAAC |
| rev | | ACTCAGAGGTCCGACCCTTAGGTACTATACGCTACTCTTGACGAACC | | ACTTAGAAGGAAGAAAGAAAAGTGAGTGTAGACCTGCATACCGATAGTT |
| | | GTGAAAATAACTTCCTCTAACACTTAGAAGGAAGAAAGAAAAGTG | | ATTACGTCCCGGAAAGGGTCCCTTAACAACCATGGTACCTTTCATCAGTTTG |
| | | AGTGTAGACCTGCATACCGATAGTTATTACGTCCCGGAAAGGGTCC | | CGTCACAAGAGTATTCGTGTCAAATATTAACTTTAGGGAGGAAAGACCTG |
| | | CTTAACAACCATGGTACCTTTCATCAGTTTGCGTCACAAGAGTATTC | | ATAGTTTCTAACTCCTGAAACAAAGTGGACGACGACAGACGCGACCAGAC |
| | | GTGTCAAATATTAACTTTAGGGAGGAAAGACCTGATAGTTTCTAAC | | AGAGGGCGAACCCAAAGTGTCTATCGAGGCAAGGGACAGGCTTCTATTCG |
| | | TCCTGAAACAAAGTGGACGACGACAGACGCGACCAGACAGAGGGC | | AAAGGAGGGACCAATATTTAGTCACGTAGTATATCCAGAACCCTGACCCT |
| | | GAACCCAAAGTGTCTATCGAGGCAAGGGACAGGCTTCTATTCGAA | | GCCGTGTACCAGCTGAGAGACTCTAAATCCAGTGACAAGTCTGTCTGCCTA |
| | | AGGAGGGACCAATATTTAGTCACGTAGTATATCCAGAACCCTGACC | | TTCACCGATTTTGATT |
| | | CTGCCGTGTACCAGCTGAGAGACTCTAAATCCAGTGACAAGTCTGT | | |
| | | CTGCCTATTCACCGATTTTGATTATTCGGAGACCTCCCGCTACTCTGG | | |
| | | CGTCGATGAGGGA | | |
| Human- | 109 | CAATCCGCCCTCACTACAACCGGATACGCCGGTCTCGAATGTAATAC | 110 | GGCACGTAGTTGTCGCGTCTTCATTCTCCGTCTCAGAAAGTAGACCTCGAG |
| TRA5_ | | GACTCACTATAGGCACGTAGTTGTCGCGTCTTCATTCTCCGTCTCAG | | GACATCTTCCTCCATTGGTATTGATAGTCTCGCTGATTCCTTACTTCTTTTTG |
| rev | | AAAGTAGACCTCGAGGACATCTTCCTCCATTGGTATTGATAGTCTCG | | CTGGACAGAGGAGGTTTGGTAGGTCTGGTAACATTCTTTCTTTACACCCAC |
| | | CTGATTCCTTACTTCTTTTTGCTGGACAGAGGAGGTTTGGTAGGTCT | | GAGGCGGTAGTACAACGACCATGGTCCTGTCGGGTATTTTGGGTCTACAG |
| | | GGTAACATTCTTTCTTTACACCCACGAGGCGGTAGTACAACGACCAT | | ACCATGCACGTCAAATAGACCTGTTACCGAGGAAGGTGTCGACAGTGAAG |
| | | GGTCCTGTCGGGTATTTTGGGTCTACAGACCATGCACGTCAAATAG | | TCTCCCGACGAGTTCCGAAACAGGGCACCAAACCATTCTAGGTGGAAATTG |
| | | ACCTGTTACCGAGGAAGGTGTCGACAGTGAAGTCTCCCGACGAGTT | | TACCAGGGGGAACGTTTCTCGTCGAAGACCGGTAGACTTTTGGCACGTAGT |
| | | CCGAAACAGGGCACCAAACCATTCTAGGTGGAAATTGTACCAGGG | | ATATCCAGAACCCTGACCCTGCCGTGTACCAGCTGAGAGACTCTAAATCCA |
| | | GGAACGTTTCTCGTCGAAGACCGGTAGACTTTTGGCACGTAGTATA | | GTGACAAGTCTGTCTGCCTATTCACCGATTTTGATT |
| | | TCCAGAACCCTGACCCTGCCGTGTACCAGCTGAGAGACTCTAAATCC | | |
| | | AGTGACAAGTCTGTCTGCCTATTCACCGATTTTGATTATTCGGAGAC | | |
| | | CTCCCGCTACTCTGGCGTCGATGAGGGA | | |
| Human- | 111 | CAATCCGCCCTCACTACAACCGGATACGCCGGTCTCGAATGTAATAC | 112 | GGCACGTAGTGGTGGCGATGACATCTCTATGTGCCGACTCAGGGAGCCGA |
| TRB1_ | | GACTCACTATAGGCACGTAGTGGTGGCGATGACATCTCTATGTGCC | | GACGCCCTACCTAGAAGTCACACCTTCTGCAAGGCAATCCAGAAAGTCGAC |
| rev | | GACTCAGGGAGCCGAGACGCCCTACCTAGAAGTCACACCTTCTGCA | | ACTTAGCAAGGAACCCGTAGGGACTTAGTTGACGTTACAAAACCATGACC |
| | | AGGCAATCCAGAAAGTCGACACTTAGCAAGGAACCCGTAGGGACT | | TTCCTTTAGTCGTTGAAGTAAAGAACGGAGTCTAAGACAGCCATGGTCACT |
| | | TAGTTGACGTTACAAAACCATGACCTTCCTTTAGTCGTTGAAGTAA | | TGTTTGTACACTGGTCTTCTACCTAGCGTGGAGTCTTAGTGGAGGACAGGG |
| | | AGAACGGAGTCTAAGACAGCCATGGTCACTTGTTTGTACACTGGTC | | TACAACCACTGGAACACAGACCCCCAGACCGACTGAGGTCCGAGAAAAAG |
| | | TTCTACCTAGCGTGGAGTCTTAGTGGAGGACAGGGTACAACCACTG | | CACGAAGACACGGGGGACAGGGGGCGCAGACACAGAGGGTCTTGGTCCT |
| | | GAACACAGACCCCCAGACCGACTGAGGTCCGAGAAAAAGCACGAA | | CAGGAGGCGGGTCTGCGGGTCGTCGCGGAGAGTCCACGTAGTGAGGACCT |
| | | GACACGGGGGACAGGGGGCGCAGACACAGAGGGTCTTGGTCCTCA | | GAACAAGGTGTTCCCACCCGAGGTCGCTGTGTTTGAGCCATCAGAAGCAG |
| | | GGAGGCGGGTCTGCGGGTCGTCGCGGAGAGTCCACGTAGTGAGGA | | AGATCTCCCACACCCAAAAGGCCACACTGGTG |
| | | CCTGAACAAGGTGTTCCCACCCGAGGTCGCTGTGTTTGAGCCATCAG | | |
| | | AAGCAGAGATCTCCCACACCCAAAAGGCCACACTGGTGATTCGGAG | | |
| | | ACCTCCCGCTACTCTGGCGTCGATGAGGGA | | |
| Human- | 113 | CAATCCGCCCTCACTACAACCGGATACGCCGGTCTCGAATGTAATAC | 114 | GGCACGTAGTCGACGACCGTGTCTCTATGTACCGGCTCAGGGGTTCGAGAC |
| TRB2_ | | GACTCACTATAGGCACGTAGTCGACGACCGTGTCTCTATGTACCGGC | | GACCGACCTAGAACTCTCACCTCAGATGAGGAAACTCGGAGAGACGTCTTT |
| rev | | TCAGGGGTTCGAGACGACCGACCTAGAACTCTCACCTCAGATGAGG | | TAGCTAGGAATCCGTTGACACTTAGCAGATGACGAAGGAGTAAGAGTAT |
| | | AAACTCGGAGAGACGTCTTTTAGCTAGGAATCCGTTGACACTTAGC | | AGCTTAGTCTTCGAGGCCCGGGACAGGGTTCAAGACGTCCATGGTCATTTC |
| | | AGATGACGAAGGAGTAAGAGTATAGCTTAGTCTTCGAGGCCCGGG | | CCATAACACCGGTCTTTAACCTAACGTGGTTTTTCGGTGTCCGACAAAGAA |
| | | ACAGGGTTCAAGACGTCCATGGTCATTTCCCATAACACCGGTCTTTA | | GAGATATTAGAATATAGACCCTCTGGGAAAGGACGGACGGGGGACAGGG |
| | | ACCTAACGTGGTTTTTCGGTGTCCGACAAAGAAGAGATATTAGAAT | | ACCGAGCCGATCCTACCTCTCAGCTCAGGGTAGTGGTTTTACGACCCCGACT |
| | | ATAGACCCTCTGGGAAAGGACGGACGGGGGACAGGGACCGAGCC | | AACGATCACGTAGTGAGGACCTGAACAAGGTGTTCCCACCCGAGGTCGCT |
| | | GATCCTACCTCTCAGCTCAGGGTAGTGGTTTTACGACCCCGACTAAC | | GTGTTTGAGCCATCAGAAGCAGAGATCTCCCACACCCAAAAGGCCACACTG |
| | | GATCACGTAGTGAGGACCTGAACAAGGTGTTCCCACCCGAGGTCGC | | GTG |
| | | TGTGTTTGAGCCATCAGAAGCAGAGATCTCCCACACCCAAAAGGCC | | |
| | | ACACTGGTGATTCGGAGACCTCCCGCTACTCTGGCGTCGATGAGGG A | | |
| Human- | 115 | CAATCCGCCCTCACTACAACCGGATACGCCGGTCTCGAATGTAATAC | 116 | GGCACGTAGTAGAACCGACGACCGCGTCTCTATGTCCCGGCTCAGAAGACC |
| TRB3_ | | GACTCACTATAGGCACGTAGTAGAACCGACGACCGCGTCTCTATGT | | GACGTCCCACACATCCACTTCCTTATTCACTCTCGACAACCCCGTAAGTCCAC |
| rev | | CCCGGCTCAGAAGACCGACGTCCCACACATCCACTTCCTTATTCACTC | | TCTTCGCTGAACCGTGTGACAACAAAAGTTGGGCAAGAAGTTCTGACATCT |
| | | TCGACAACCCCGTAAGTCCACTCTTCGCTGAACCGTGTGACAACAAA | | GTTTGTACTCGAGGTCACCGAAGAATCGTGAAACGAACATGGTTATGTAT |
| | | AGTTGGGCAAGAAGTTCTGACATCTGTTTGTACTCGAGGTCACCGA | | CGCAATACTGGGTCTACAACAAGTGTAAAGTTTCTGAAGAATAAACAGTA |
| | | AGAATCGTGAAACGAACATGGTTATGTATCGCAATACTGGGTCTAC | | AGGGTACTGGTCCACAGAACCACAGACGCATTGAGGGCAAAGCCAGCCGG |
| | | AACAAGTGTAAAGTTTCTGAAGAATAAACAGTAAGGGTACTGGTC | | CCGCGGGAGGGCGATCAGGGCAGGGACCAGGATCCTACCTCTCAGCTCAG |
| | | CACAGAACCACAGACGCATTGAGGGCAAAGCCAGCCGGCCGCGGG | | GGTAGTGGTTTTACGACCCCGACTAACGATCACGTAGTGAGGACCTGAAC |
| | | AGGGCGATCAGGGCAGGGACCAGGATCCTACCTCTCAGCTCAGGGT | | AAGGTGTTCCCACCCGAGGTCGCTGTGTTTGAGCCATCAGAAGCAGAGATC |
| | | AGTGGTTTTACGACCCCGACTAACGATCACGTAGTGAGGACCTGAA | | TCCCACACCCAAAAGGCCACACTGGTG |
| | | CAAGGTGTTCCCACCCGAGGTCGCTGTGTTTGAGCCATCAGAAGCA | | |
| | | GAGATCTCCCACACCCAAAAGGCCACACTGGTGATTCGGAGACCTC | | |
| | | CCGCTACTCTGGCGTCGATGAGGGA | | |
| Human- | 117 | CAATCCGCCCTCACTACAACCGGATACGCCGGTCTCGAATGTAATAC | 118 | GGCACGTAGTTCAAGGGGTTCTCGTGTTTTCATGAAACGACTTAGAAGATC |
| TRD4_ | | GACTCACTATAGGCACGTAGTTCAAGGGGTTCTCGTGTTTTCATGA | | GACATTCCGACTTTACCAATTCCGCTGCCTAAAGCGACGAAAGAACTTCAA |
| rev | | AACGACTTAGAAGATCGACATTCCGACTTTACCAATTCCGCTGCCTA | | CTGTCTTATCGCTGGTGAAAAACGTAAGACAAGTAGTCTTGGGACCGCTTA |
| | | AAGCGACGAAAGAACTTCAACTGTCTTATCGCTGGTGAAAAACGT | | TTCCTTTTAGTAGAGAAACGACCCTTCAACGAACATGGTTTTTTATATTATA |
| | | AAGACAAGTAGTCTTGGGACCGCTTATTCCTTTTAGTAGAGAAACG | | CTGGTGGTTGAACAAAGTATGTCCGTCAAGTCCCACTGACGAAAGGAGTG |
| | | ACCCTTCAACGAACATGGTTTTTTATATTATACTGGTGGTTGAACAA | | ACCGTACCTATGACTACTGACCCGAACTCATTGGAAGACCCGCGACAAGTG |
| | | AGTATGTCCGTCAAGTCCCACTGACGAAAGGAGTGACCGTACCTAT | | ATAAAGACCACCCCCGAGGTGCTTCTCAAACTACGGTCAAGGCTTTTTGTA |
| | | GACTACTGACCCGAACTCATTGGAAGACCCGCGACAAGTGATAAA | | GACAGCCCACAGGGTCCTCCACGTAGTGAAGTCAGCCTCATACCAAACCAT |
| | | GACCACCCCCGAGGTGCTTCTCAAACTACGGTCAAGGCTTTTTGTAG | | CCGTTTTTGTCATGAAAAATGGAACAAATGTCGCTTGTCTGGTGAAGGAA |
| | | ACAGCCCACAGGGTCCTCCACGTAGTGAAGTCAGCCTCATACCAAA | | TTCTACCCCAAGGA |
| | | CCATCCGTTTTTGTCATGAAAAATGGAACAAATGTCGCTTGTCTGGT | | |
| | | GAAGGAATTCTACCCCAAGGAATTCGGAGACCTCCCGCTACTCTGG | | |
| | | CGTCGATGAGGGA | | |
| Human- | 119 | CAATCCGCCCTCACTACAACCGGATACGCCGGTCTCGAATGTAATAC | 120 | GGCACGTAGTTCAAGGGGTTCTCGTGTTTTCATGAAACGACTTAGAAGATC |
| TRD5_ | | GACTCACTATAGGCACGTAGTTCAAGGGGTTCTCGTGTTTTCATGA | | GACATTCCGACTTTACCAATTCCGCTGCCTAAAGCGACGAAAGAACTTCAA |
| rev | | AACGACTTAGAAGATCGACATTCCGACTTTACCAATTCCGCTGCCTA | | CTGTCTTATCGCTGGTGAAAAACGTAAGACAAGTAGTCTTGGGACCGCTTA |
| | | AAGCGACGAAAGAACTTCAACTGTCTTATCGCTGGTGAAAAACGT | | TTCCTTTTAGTAGAGAAACGACCCTTCAACGAACATGGTTTTTTATATTATA |
| | | AAGACAAGTAGTCTTGGGACCGCTTATTCCTTTTAGTAGAGAAACG | | CTGGTGGTTGAACAAAGTATGTCCGTCAAGTCCCACTGACGAAAGGAGTG |
| | | ACCCTTCAACGAACATGGTTTTTTATATTATACTGGTGGTTGAACAA | | ACCGTACCTATGACTACTGACCCGAACTCATTGGAAGACCCGAAGGCAGAC |
| | | AGTATGTCCGTCAAGTCCCACTGACGAAAGGAGTGACCGTACCTAT | | CGCCAAGGTGATAAAGAACAACGCCGACCAAGGTGCTACTCAACACAAGG |
| | | GACTACTGACCCGAACTCATTGGAAGACCCGAAGGCAGACCGCCAA | | GAAAGGTTTCTTCTCAACACGACAGTTTCCACGTAGTGAAGTCAGCCTCAT |
| | | GGTGATAAAGAACAACGCCGACCAAGGTGCTACTCAACACAAGGG | | ACCAAACCATCCGTTTTTGTCATGAAAAATGGAACAAATGTCGCTTGTCTG |
| | | AAAGGTTTCTTCTCAACACGACAGTTTCCACGTAGTGAAGTCAGCCT | | GTGAAGGAATTCTACCCCAAGGA |
| | | CATACCAAACCATCCGTTTTTGTCATGAAAAATGGAACAAATGTCG | | |
| | | CTTGTCTGGTGAAGGAATTCTACCCCAAGGAATTCGGAGACCTCCC | | |
| | | GCTACTCTGGCGTCGATGAGGGA | | |
| Human- | 121 | CAATCCGCCCTCACTACAACCGGATACGCCGGTCTCGAATGTAATAC | 122 | GGCACGTAGTTCAAGGGGTTCTCGTGTTTTCATGAAACGACTTAGAAGATC |
| TRD6_ | | GACTCACTATAGGCACGTAGTTCAAGGGGTTCTCGTGTTTTCATGA | | GACATTCCGACTTTACCAATTCCGCTGCCTAAAGCGACGAAAGAACTTCAA |
| rev | | AACGACTTAGAAGATCGACATTCCGACTTTACCAATTCCGCTGCCTA | | CTGTCTTATCGCTGGTGAAAAACGTAAGACAAGTAGTCTTGGGACCGCTTA |
| | | AAGCGACGAAAGAACTTCAACTGTCTTATCGCTGGTGAAAAACGT | | TTCCTTTTAGTAGAGAAACGACCCTTCAACGAACATGGTTTTTTATATTATA |
| | | AAGACAAGTAGTCTTGGGACCGCTTATTCCTTTTAGTAGAGAAACG | | CTGGTGGTTGAACAAAGTATGTCCGTCAAGTCCCACTGACGAAAGGAGTG |
| | | ACCCTTCAACGAACATGGTTTTTTATATTATACTGGTGGTTGAACAA | | ACCGTACCTATGACTACTGACCCGAACTCATTGGAAGACCCGAGCCGATGA |
| | | AGTATGTCCGTCAAGTCCCACTGACGAAAGGAGTGACCGTACCTAT | | TAAAGACAGAACAACAACATGGAGGTCTATCCAAGGAAACGGTTTCTAGT |
| | | GACTACTGACCCGAACTCATTGGAAGACCCGAGCCGATGATAAAG | | CCCCAGACCCACGTAGTGAAGTCAGCCTCATACCAAACCATCCGTTTTTGTC |
| | | ACAGAACAACAACATGGAGGTCTATCCAAGGAAACGGTTTCTAGT | | ATGAAAAATGGAACAAATGTCGCTTGTCTGGTGAAGGAATTCTACCCCAA GGA |
| | | CCCCAGACCCACGTAGTGAAGTCAGCCTCATACCAAACCATCCGTTT | | |
| | | TTGTCATGAAAAATGGAACAAATGTCGCTTGTCTGGTGAAGGAAT | | |
| | | TCTACCCCAAGGAATTCGGAGACCTCCCGCTACTCTGGCGTCGATGA | | |
| | | GGGA | | |
| Human- | 123 | CAATCCGCCCTCACTACAACCGGATACGCCGGTCTCGAATGTAATAC | 124 | GGCACGTAGTGTCCCGGGTCCAACACGTCATCAAGTACGGGAGCTACGGA |
| TRG4_ | | GACTCACTATAGGCACGTAGTGTCCCGGGTCCAACACGTCATCAAG | | CAGAGGTTGAAGTCATGACGGTTCTATCTCGACGACGGTAGGAACCGATA |
| rev | | TACGGGAGCTACGGACAGAGGTTGAAGTCATGACGGTTCTATCTCG | | CTAAAATAGACGAAAGTCCTACCTTAGGGTGACGTGTAGGTTGAACCTGT |
| | | ACGACGGTAGGAACCGATACTAAAATAGACGAAAGTCCTACCTTA | | ACCGGTCGACCACCTCACGGAGTCCCTGGACCGGGAAGACGACCATGGTCA |
| | | GGGTGACGTGTAGGTTGAACCTGTACCGGTCGACCACCTCACGGAG | | CCCACATCGACTAGGACCACGCCTGGTGCGTGACGTCCTACTGGTTCGAAG |
| | | TCCCTGGACCGGGAAGACGACCATGGTCACCCACATCGACTAGGAC | | GGATCACGGGGTCCTGTACCCGGTCGACGAGGCCGGACTACTCCAGAAAG |
| | | CACGCCTGGTGCGTGACGTCCTACTGGTTCGAAGGGATCACGGGGT | | GAACACCGGGGAAGGCAGAAGGACACTGTTGTTCACAACAAGGTGACGG |
| | | CCTGTACCCGGTCGACGAGGCCGGACTACTCCAGAAAGGAACACCG | | TTTCTCAAAGAATATTATTAAGCACGTAGTGATAAACAACTTGATGCAGA |
| | | GGGAAGGCAGAAGGACACTGTTGTTCACAACAAGGTGACGGTTTC | | TGTTTCCCCCAAGCCCACTATTTTTCTTCCTTCGATTGCTGAAACAAAACTCC |
| | | TCAAAGAATATTATTAAGCACGTAGTGATAAACAACTTGATGCAG | | AGAAGGCTGGAACATAC |
| | | ATGTTTCCCCCAAGCCCACTATTTTTCTTCCTTCGATTGCTGAAACAA | | |
| | | AACTCCAGAAGGCTGGAACATACATTCGGAGACCTCCCGCTACTCT | | |
| | | GGCGTCGATGAGGGA | | |
| Human- | 125 | CAATCCGCCCTCACTACAACCGGATACGCCGGTCTCGAATGTAATAC | 126 | GGCACGTAGTCACGGATTAGGTCGTCCGTGTCACCATGTGGTGGAGTAGA |
| TRG6_ | | GACTCACTATAGGCACGTAGTCACGGATTAGGTCGTCCGTGTCACC | | AGAAAGAGATTCTTGAAATAAAAGTTTCACCTTCACCTTCACACTCGTAAA |
| rev | | ATGTGGTGGAGTAGAAGAAAGAGATTCTTGAAATAAAAGTTTCAC | | AATGAATAGAGTTCAAAGAATCAGAAGGGTGGACTCGTTAGAACTCGTCT |
| | | CTTCACCTTCACACTCGTAAAAATGAATAGAGTTCAAAGAATCAGA | | CTAACAGTTCTTCTGTACATTATTTATAAGATTCGGAAACAAACCAAAGAC |
| | | AGGGTGGACTCGTTAGAACTCGTCTCTAACAGTTCTTCTGTACATTA | | GGTCATGGTCACATACTAAAATGACGATTTCGGAACCTACCTACGGAAGG |
| | | TTTATAAGATTCGGAAACAAACCAAAGACGGTCATGGTCACATACT | | TTCTATACACCCGTGAGAATAAACGACCAGACCTTTATCTATAAAGTCCAA |
| | | AAAATGACGATTTCGGAACCTACCTACGGAAGGTTCTATACACCCG | | CAAGGTTGACGGGTTCGAACGCCACGGGAAGGAGCCCACGCCGAAGACAC |
| | | TGAGAATAAACGACCAGACCTTTATCTATAAAGTCCAACAAGGTTG | | TGTTGTTCACAACAAGGTGACGGTTTCTCAAAGAATATTATTAAGCACGTA |
| | | ACGGGTTCGAACGCCACGGGAAGGAGCCCACGCCGAAGACACTGT | | GTGATAAACAACTTGATGCAGATGTTTCCCCCAAGCCCACTATTTTTCTTCC |
| | | TGTTCACAACAAGGTGACGGTTTCTCAAAGAATATTATTAAGCACG | | TTCGATTGCTGAAACAAAACTCCAGAAGGCTGGAACATAC |
| | | TAGTGATAAACAACTTGATGCAGATGTTTCCCCCAAGCCCACTATTT | | |
| | | TTCTTCCTTCGATTGCTGAAACAAAACTCCAGAAGGCTGGAACATA | | |
| | | CATTCGGAGACCTCCCGCTACTCTGGCGTCGATGAGGGA | | |
| Human- | 127 | CAATCCGCCCTCACTACAACCGGATACGCCGGTCTCGAATGTAATAC | 128 | GGCACGTAGTCACGGATTAGGTCGTCCGTGTCACCATGTGGTGGAGTAGA |
| TRG7_ | | GACTCACTATAGGCACGTAGTCACGGATTAGGTCGTCCGTGTCACC | | AGAAAGAGATTCTTGAAATAAAAGTTTCACCTTCACCTTCACACTCGTAAA |
| rev | | ATGTGGTGGAGTAGAAGAAAGAGATTCTTGAAATAAAAGTTTCAC | | AATGAATAGAGTTCAAAGAATCAGAAGGGTGGACTCGTTAGAACTCGTCT |
| | | CTTCACCTTCACACTCGTAAAAATGAATAGAGTTCAAAGAATCAGA | | CTAACAGTTCTTCTGTACATTATTTATAAGATTCGGAAACAAACCAAAGAC |
| | | AGGGTGGACTCGTTAGAACTCGTCTCTAACAGTTCTTCTGTACATTA | | GGTCATGGTCACATACTAAAATGACGATTTCGGAACCTACCTACGGAAGG |
| | | TTTATAAGATTCGGAAACAAACCAAAGACGGTCATGGTCACATACT | | TTCTATACACCCGTGAGAATAAACGACCAGACCTTTATCTATAAAGTCCAA |
| | | AAAATGACGATTTCGGAACCTACCTACGGAAGGTTCTATACACCCG | | CAAGGTTGACGGGTTCGCACCCGAAAAAGCCCACGCCAGCGCAGAACGTC |
| | | TGAGAATAAACGACCAGACCTTTATCTATAAAGTCCAACAAGGTTG | | CGCTTCAATGATACTCGGATCAGGGAAAACGTTTGCAGAACTAGGTTAGT |
| | | ACGGGTTCGCACCCGAAAAAGCCCACGCCAGCGCAGAACGTCCGCT | | GATGATACACGTAGTGATAAACAACTTGATGCAGATGTTTCCCCCAAGCCC |
| | | TCAATGATACTCGGATCAGGGAAAACGTTTGCAGAACTAGGTTAG | | ACTATTTTTCTTCCTTCGATTGCTGAAACAAAACTCCAGAAGGCTGGAACA TAC |
| | | TGATGATACACGTAGTGATAAACAACTTGATGCAGATGTTTCCCCC | | |
| | | AAGCCCACTATTTTTCTTCCTTCGATTGCTGAAACAAAACTCCAGAA | | |
| | | GGCTGGAACATACATTCGGAGACCTCCCGCTACTCTGGCGTCGATG | | |
| | | AGGGA | | |

1. A synthetic nucleic acid oligonucleotide, comprising (a) one or more tags with a sequence that is not contained in a eukaryotic immune repertoire wherein at least one tag is positioned between a region corresponding to a diversity and joining region ((D)J) and a constant region (C) of an immunoglobulin (IG) or T cell receptor thereby distinguishing the synthetic oligonucleotide from naturally occurring sequences in the eukaryotic immune repertoire; and wherein the V(D)J region has a predetermined V(D)J clonotype, the synthetic oligonucleotide optionally further comprising a tag proximate to a region corresponding to a eukaryotic variable region (V); and optionally wherein C further comprises one or more priming sites for nucleic acid amplification.

2. The synthetic oligonucleotide of claim 1, wherein the oligonucleotide is a DNA oligonucleotide.

3. The DNA oligonucleotide of claim 2, wherein the DNA is selected from a single stranded DNA or a double stranded DNA.

4. The DNA oligonucleotide of claim 2, further comprising a promoter sequence for a DNA dependent RNA polymerase, optionally wherein one of the tags is positioned between the promoter sequence and the variable region.

5. (canceled)

6. The DNA oligonucleotide of claim 2, further comprising a poly dT tail.

7. The synthetic oligonucleotide of claim 1, wherein the oligonucleotide has a length in the range of 200-1000 nucleotides, and optionally in the range of 450-700 nucleotides.

8. The synthetic oligonucleotide of claim 1, wherein the tag comprises a number of nucleotides in the range of 6-25 nucleotides, and optionally comprises 8 nucleotides.

9. The synthetic oligonucleotide of claim 1, wherein the oligonucleotide is an RNA oligonucleotide.

10. The RNA oligonucleotide of claim 9, further comprising a poly A tail.

11. The DNA oligonucleotide of claim 2, wherein the DNA oligonucleotide is inserted into a bacterial plasmid.

12. The DNA oligonucleotide of claim 2, further comprising a priming site for nucleic acid amplification.

13. The synthetic oligonucleotide of claim 1, further comprising sequencing adaptors.

14. The synthetic oligonucleotide of claim 1, wherein the eukaryotic immune repertoire is a mammalian immune repertoire.

15. The synthetic oligonucleotide of claim 1, wherein, the sequence of the V(D)J region comprises naturally occurring sequence, non-naturally occurring sequence, or a combination thereof.

16. (canceled)

17. The synthetic oligonucleotide of claim 1, wherein the synthetic oligonucleotide is contained within a mixture of synthetic oligonucleotides wherein each oligonucleotide in the mixture contains a different V(D)J clonotype.

18. The synthetic oligonucleotide of claim 17, wherein the mixture of clonotypes comprises each of at least two different clonotypes.

19. The synthetic oligonucleotide of claim 18, wherein a plurality of mixtures of clonotypes are combined to form an internal control for a eukaryotic derived sample, optionally wherein the plurality of mixtures of clonotypes comprises at least two mixtures for forming a control pool.

20. (canceled)

21. A method for RNA analysis of an immune response, comprising:
(a) obtaining a synthetic oligonucleotide of claim 1;
(b) optionally performing in vitro transcription of the synthetic oligonucleotide if the synthetic oligonucleotide is DNA and omitting this step if the synthetic oligonucleotide is RNA;
(c) optionally performing a capping reaction if the synthetic oligonucleotide is RNA; or
optionally performing a capping reaction on the transcribed RNA if the synthetic oligonucleotide is DNA;
(d) forming a control pool of RNA oligonucleotides from the DNA oligonucleotides in (b) or from the RNA oligonucleotides in (a) or (c) wherein the control pool of RNA oligonucleotides comprises a plurality of mixtures of clonotypes wherein each mixture comprises a plurality of clonotypes at different predetermined concentrations;
(e) adding a predetermined concentration of the control pool of RNA from (d) to a sample obtained from a eukaryote RNAs produced by an immune response;
(f) preparing a library of (e) for sequencing, and optionally sequencing the library.

22. The method of claim 21, wherein (a) further comprises adding a polynucleotide tail to the synthetic oligonucleotide by means of a primer and amplifying the primed oligonucleotide.

23. (canceled)

24. The method of claim 21, further comprising identifying the control pool of the synthetic oligonucleotides, and optionally using the identified control pool for a purpose selected from quantifying the eukaryotic RNA associated with immune response; normalizing the eukaryotic RNA associated with immune response; determining the sensitivity of an immune repertoire technology; and determining the sensitivity of an immune repertoire technology.

25. (canceled)
